SEQUENCE LISTING (174 sequences)

SEQ ID NO: 1 - DNA, 41 bp, Adeno-Associated Virus
  primer_bind (1)..(41)
caaccactacttcggctacgccaccccatgggggtattttg

SEQ ID NO: 2 - DNA, 42 bp, Adeno-Associated Virus
  primer_bind (1)..(42)
caaaaacagacaacaacaacgccaattttacctggactggtg

SEQ ID NO: 3 - DNA, 29 bp, Adeno-Associated Virus
  primer_bind (1)..(29)
gcactgccatggccgcacacaaagacgac

SEQ ID NO: 4 - DNA, 40 bp, Adeno-Associated Virus
  primer_bind (1)..(40)
cgaatcctcccgcggagtttgcagctacaaagtttgcttc

SEQ ID NO: 5 - DNA, 41 bp, Adeno-Associated Virus
  primer_bind (1)..(41)
tttcagctacaaagtttgctgcattcatcacccaatactcc

SEQ ID NO: 6 - DNA, 30 bp, Adeno-Associated Virus
  primer_bind (1)..(30)
cctgggccttgccggcctacaataaccacc

SEQ ID NO: 7 - DNA, 40 bp, Adeno-Associated Virus
  primer_bind (1)..(40)
ctggttgaggaaggcgcgagaacggctcctggaaagaaac

SEQ ID NO: 8 - DNA, 42 bp, Adeno-Associated Virus
  primer_bind (1)..(42)
cactactttggctacgccaccccttgggggtattttgacttc

SEQ ID NO: 9 - DNA, 34 bp, Adeno-Associated Virus
  primer_bind (1)..(34)
taccgccagcagcgagtagctaagacatctgcgg

SEQ ID NO: 10 - DNA, 42 bp, Adeno-Associated Virus
  primer_bind (1)..(42)
aagacatctgcagataacaacaacgctgaatactcgtggact

SEQ ID NO: 11 - DNA, 31 bp, Adeno-Associated Virus
  primer_bind (1)..(31)
ccgggcccggcgatggcagcccacaaggacg

SEQ ID NO: 12 - DNA, 40 bp, Adeno-Associated Virus
  primer_bind (1)..(40)
agaaaagttttttcctcaggccggggttctcatctttggg

SEQ ID NO: 13 - DNA, 41 bp, Adeno-Associated Virus
  primer_bind (1)..(41)
catctttgggaagcaaggcgccgagaaaacaaatgtggaca

SEQ ID NO: 14 - DNA, 38 bp, Adeno-Associated Virus
  primer_bind (1)..(38)
aatccttcgaccaccttcgctgcggcaaagtttgcttc

SEQ ID NO: 15 - DNA, 51 bp, Adeno-Associated Virus
  primer_bind (1)..(51)
gcgaatccgtcgaccaccttcagtgcggcaaagtttgctgccttcatcaca

SEQ ID NO: 16 - DNA, 42 bp, Adeno-Associated Virus
  primer_bind (1)..(42)
acccgaacctgggccctgccggcctacaacaaccacctctac

SEQ ID NO: 17 - DNA, 51 bp, Adeno-Associated Virus
  primer_bind (1)..(51)
tattacttaagcagaacaaacactccaagtggagccaccacgcagtcaagg

SEQ ID NO: 18 - DNA, 51 bp, Adeno-Associated Virus
  primer_bind (1)..(51)
acatctgcagataacaacaacagtgaatactcgtgggctggagctaccaag

SEQ ID NO: 19 - DNA, 48 bp, Adeno-Associated Virus
  primer_bind (1)..(48)
ttcagtgcggccaagtttgcttccttcatcgcacagtactccacggga

SEQ ID NO: 20 - DNA, 48 bp, Adeno-Associated Virus
  primer_bind (1)..(48)
cgctggaatcccgaaattcagtacgcttccaactacaacaagtctgtt

SEQ ID NO: 21 - DNA, 45 bp, Adeno-Associated Virus
  primer_bind (1)..(45)
gttaatgtggactttgctgtcgacactaatggcgtgtattcagag

SEQ ID NO: 22 - DNA, 48 bp, Adeno-Associated Virus
  primer_bind (1)..(48)
aagtctgttaatgtagactttactgtggacgctaatggcgtgtattca

SEQ ID NO: 23 - DNA, 45 bp, Adeno-Associated Virus
  primer_bind (1)..(45)
ccaaagcccgcagaacggcatagggacgacagcaggggtcttgtg

SEQ ID NO: 24 - DNA, 45 bp, Adeno-Associated Virus
  primer_bind (1)..(45)
ggcctggttgaggaacctgttaggacggccccgggaaaaaagagg

SEQ ID NO: 25 - DNA, 42 bp, Adeno-Associated Virus
  primer_bind (1)..(42)
cctgttaagacggccccgggaaaaaggaggccggtagagcac

SEQ ID NO: 26 - DNA, 54 bp, Adeno-Associated Virus
  primer_bind (1)..(54)
gactcctcctcgggaaccggaagggccggccagcagcctgcaagaaaaagattg

SEQ ID NO: 27 - DNA, 39 bp, Adeno-Associated Virus
  primer_bind (1)..(39)
cagcagcgagtatcaaggacatctgcagataacaacaac

SEQ ID NO: 28 - DNA, 39 bp, Adeno-Associated Virus
  primer_bind (1)..(39)
tggactggagctaccagataccacctcaatggcagagac

SEQ ID NO: 29 - DNA, 42 bp, Adeno-Associated Virus
  primer_bind (1)..(42)
ccgggcccggcaatggcaagccacagggacgatgaagaaaag

SEQ ID NO: 30 - DNA, 45 bp, Adeno-Associated Virus
  primer_bind (1)..(45)
cacaaggacgatgaagaaaggttttttcctcaaagcggggttctc

SEQ ID NO: 31 - DNA, 42 bp, Adeno-Associated Virus
  primer_bind (1)..(42)
gttctcatctttggtagacaaggctcagagaaaacaaatgtg

SEQ ID NO: 32 - DNA, 30 bp, Adeno-Associated Virus
  primer_bind (1)..(30)
cctgggccctgcccgcttacaacaaccatc

SEQ ID NO: 33 - DNA, 29 bp, Adeno-Associated Virus
  primer_bind (1)..(29)
cctgggtcttgcccgcctacaacaaccac

SEQ ID NO: 34 - DNA, 30 bp, Adeno-Associated Virus
  primer_bind (1)..(30)
gcctactttggatacgccaccccctggggg

SEQ ID NO: 35 - DNA, 28 bp, Adeno-Associated Virus
  primer_bind (1)..(28)
ggtcaaccgcgccgctgtcagcgccttc

SEQ ID NO: 36 - DNA, 29 bp, Adeno-Associated Virus
  primer_bind (1)..(29)
atatcaccagcttcgcggacgtgcccgtc

SEQ ID NO: 37 - DNA, 32 bp, Adeno-Associated Virus
  primer_bind (1)..(32)
gacgtgcccgtcagcgccttcatcacccagta

SEQ ID NO: 38 - DNA, 29 bp, Adeno-Associated Virus
  primer_bind (1)..(29)
cgggcccaccgaaaccaagacccaatcag

SEQ ID NO: 39 - DNA, 25 bp, Adeno-Associated Virus
  primer_bind (1)..(25)
ctcgccgacgacgcatccttcgggg

SEQ ID NO: 40 - DNA, 27 bp, Adeno-Associated Virus
  primer_bind (1)..(27)
cgccaacaacctcgcctccaccgtcca

SEQ ID NO: 41 - DNA, 28 bp, Adeno-Associated Virus
  primer_bind (1)..(28)
catgggccttgccggcctataacaacca

SEQ ID NO: 42 - DNA, 27 bp, Adeno-Associated Virus
  primer_bind (1)..(27)
ctgggccctgcccgcctacaacaacca

SEQ ID NO: 43 - DNA, 33 bp, Adeno-Associated Virus
  primer_bind (1)..(33)
gagaccggtagagccagcaccccagcgttctcc

SEQ ID NO: 44 - DNA, 35 bp, Adeno-Associated Virus
  primer_bind (1)..(35)
cccagcgttctccagacgcctctacgggcatcggc

SEQ ID NO: 45 - DNA, 28 bp, Adeno-Associated Virus
  primer_bind (1)..(28)
ctacttcggctacgccaccccctggggg

SEQ ID NO: 46 - DNA, 40 bp, Adeno-Associated Virus
  primer_bind (1)..(40)
gacaaccggtcaaaacaacaatgccaactttgcctggact

SEQ ID NO: 47 - DNA, 42 bp, Adeno-Associated Virus
  primer_bind (1)..(42)
ccttcaaccagtcaaagcagaacgctttcatcacgcaataca

SEQ ID NO: 48 - DNA, 35 bp, Adeno-Associated Virus
  primer_bind (1)..(35)
ttgaggaaggcgctaaggcggctcctggaaagaag

SEQ ID NO: 49 - DNA, 42 bp, Adeno-Associated Virus; identical to SEQ ID NO: 16
  primer_bind (1)..(42)

SEQ ID NO: 50 - DNA, 40 bp, Adeno-Associated Virus
  primer_bind (1)..(40)
agatcctgatcaagaacgcgccagtacctgcggatcctcc

SEQ ID NO: 51 - DNA, 39 bp, Adeno-Associated Virus
  primer_bind (1)..(39)
aaagctgaactctttcatcgcgcaatacagcaccggaca

SEQ ID NO: 52 - DNA, 30 bp, Adeno-Associated Virus
  primer_bind (1)..(30)
ggttgaggaaggggctaggacggctcctgg

SEQ ID NO: 53 - DNA, 43 bp, Adeno-Associated Virus
  primer_bind (1)..(43)
aggcgctaagacggctcctggaaagagaagaccggtagagcca

SEQ ID NO: 54 - DNA, 56 bp, Adeno-Associated Virus
  primer_bind (1)..(56)
tggcctgaaacatcctccgcctcagatcctgattcgaaacacgcctgtacctgcgg

SEQ ID NO: 55 - DNA, 41 bp, Adeno-Associated Virus
  primer_bind (1)..(41)
caacgcctacttcggctacgcaaccccctgggggtattttg

SEQ ID NO: 56 - DNA, 47 bp, Adeno-Associated Virus
  primer_bind (1)..(47)
cagctaccgacaacaacgtgtcgcgaccactgtgactcaaaacaaca

SEQ ID NO: 57 - DNA, 54 bp, Adeno-Associated Virus
  primer_bind (1)..(54)
gccttcaacaaggacaagctgaacgcctttatcacccagtattctactggccaa

SEQ ID NO: 58 - DNA, 49 bp, Adeno-Associated Virus
  primer_bind (1)..(49)
ccactgtgactcaaaacaacaacgccgagtttgcttggcctggagcttc

SEQ ID NO: 59 - DNA, 40 bp, Adeno-Associated Virus
  primer_bind (1)..(40)
cccgaacctgggccctgccagcctacaacaatcacctcta

SEQ ID NO: 60 - DNA, 43 bp, Adeno-Associated Virus
  primer_bind (1)..(43)
agcggctaagacggctcctggaaagagaaggcctgtagagcag

SEQ ID NO: 61 - DNA, 30 bp, Adeno-Associated Virus
  primer_bind (1)..(30)
gcctacgaccagcagctcagagcgggtgac

SEQ ID NO: 62 - DNA, 28 bp, Adeno-Associated Virus
  primer_bind (1)..(28)
ggttgaggaaggcgctaggacggctcct

SEQ ID NO: 63 - DNA, 35 bp, Adeno-Associated Virus
  primer_bind (1)..(35)
gcagaatgaaggcaccaggaccatcgccaataacc

SEQ ID NO: 64 - DNA, 27 bp, Adeno-Associated Virus
  primer_bind (1)..(27)
gcagcaacgcgtcgccacgacactgtc

SEQ ID NO: 65 - DNA, 38 bp, Adeno-Associated Virus
  primer_bind (1)..(38)
ctgtcgcaaaataacaacgccaactttgcctggaccgg

SEQ ID NO: 66 - DNA, 31 bp, Adeno-Associated Virus
  primer_bind (1)..(31)
caagctaagctggcggcgttcatcacgcagt

SEQ ID NO: 67 - DNA, 31 bp, Adeno-Associated Virus
  primer_bind (1)..(31)
gcgtctgcaagaagatgcgtcttttgggggc

SEQ ID NO: 68 - DNA, 27 bp, Adeno-Associated Virus
  primer_bind (1)..(27)
ctgggccctccccgcctacaacaacca

SEQ ID NO: 69 - DNA, 29 bp, Adeno-Associated Virus
  primer_bind (1)..(29)
tggcgtcgttcatcgcgcagtacagcacc

SEQ ID NO: 70 - DNA, 41 bp, Adeno-Associated Virus
  primer_bind (1)..(41)
caaccactacttcggctacagcaccccctgggggtattttg

SEQ ID NO: 71 - DNA, 42 bp, Adeno-Associated Virus
  primer_bind (1)..(42)
caaaaacagacaacaacaacagcaattttacctggactggtg

SEQ ID NO: 72 - DNA, 29 bp, Adeno-Associated Virus
  primer_bind (1)..(29)
gcactgctatggcctcacacaaagacgac

SEQ ID NO: 73 - DNA, 40 bp, Adeno-Associated Virus
  primer_bind (1)..(40)
cgaatcctccggcggagttttcagctacaaagtttgcttc

SEQ ID NO: 74 - DNA, 41 bp, Adeno-Associated Virus
  primer_bind (1)..(41)
tttcagctacaaagtttgcttcattcatcacccaatactcc

SEQ ID NO: 75 - DNA, 30 bp, Adeno-Associated Virus
  primer_bind (1)..(30)
cctgggccttgcccacctacaataaccacc

SEQ ID NO: 76 - DNA, 40 bp, Adeno-Associated Virus
  primer_bind (1)..(40)
ctggttgaggaaggcgctaagacggctcctggaaagaaac

SEQ ID NO: 77 - DNA, 42 bp, Adeno-Associated Virus
  primer_bind (1)..(42)
cactactttggctacagcaccccttgggggtattttgacttc

SEQ ID NO: 78 - DNA, 34 bp, Adeno-Associated Virus
  primer_bind (1)..(34)
taccgccagcagcgagtatcaaagacatctgcgg

SEQ ID NO: 79 - DNA, 42 bp, Adeno-Associated Virus
  primer_bind (1)..(42)
aagacatctgcggataacaacaacagtgaatactcgtggact

SEQ ID NO: 80 - DNA, 31 bp, Adeno-Associated Virus
  primer_bind (1)..(31)
ccgggcccggccatggcaagccacaaggacg

SEQ ID NO: 81 - DNA, 40 bp, Adeno-Associated Virus
  primer_bind (1)..(40)
agaaaagttttttcctcagagcggggttctcatctttggg

SEQ ID NO: 82 - DNA, 41 bp, Adeno-Associated Virus
  primer_bind (1)..(41)
catctttgggaagcaaggctcagagaaaacaaatgtggaca

SEQ ID NO: 83 - DNA, 38 bp, Adeno-Associated Virus
  primer_bind (1)..(38)
aatccttcgaccaccttcagtgcggcaaagtttgcttc

SEQ ID NO: 84 - DNA, 51 bp, Adeno-Associated Virus
  primer_bind (1)..(51)
gcgaatccttcgaccaccttcagtgcggcaaagtttgcttccttcatcaca

SEQ ID NO: 85 - DNA, 42 bp, Adeno-Associated Virus
  primer_bind (1)..(42)
acccgaacctgggccctgcccacctacaacaaccacctctac

SEQ ID NO: 86 - DNA, 51 bp, Adeno-Associated Virus
  primer_bind (1)..(51)
tattacttgagcagaacaaacactccaagtggaaccaccacgcagtcaagg

SEQ ID NO: 87 - DNA, 51 bp, Adeno-Associated Virus
  primer_bind (1)..(51)
acatctgcggataacaacaacagtgaatactcgtggactggagctaccaag

SEQ ID NO: 88 - DNA, 48 bp, Adeno-Associated Virus
  primer_bind (1)..(48)
ttcagtgcggcaaagtttgcttccttcatcacacagtactccacggga

SEQ ID NO: 89 - DNA, 48 bp, Adeno-Associated Virus
  primer_bind (1)..(48)
cgctggaatcccgaaattcagtacacttccaactacaacaagtctgtt

SEQ ID NO: 90 - DNA, 45 bp, Adeno-Associated Virus
  primer_bind (1)..(45)
gttaatgtggactttactgtggacactaatggcgtgtattcagag

SEQ ID NO: 91 - DNA, 48 bp, Adeno-Associated Virus
  primer_bind (1)..(48)
aagtctgttaatgtggactttactgtggacactaatggcgtgtattca

SEQ ID NO: 92 - DNA, 45 bp, Adeno-Associated Virus
  primer_bind (1)..(45)
ccaaagcccgcagagcggcataaggacgacagcaggggtcttgtg

SEQ ID NO: 93 - DNA, 45 bp, Adeno-Associated Virus
  primer_bind (1)..(45)
ggcctggttgaggaacctgttaagacggctccgggaaaaaagagg

SEQ ID NO: 94 - DNA, 42 bp, Adeno-Associated Virus
  primer_bind (1)..(42)
cctgttaagacggctccgggaaaaaagaggccggtagagcac

SEQ ID NO: 95 - DNA, 54 bp, Adeno-Associated Virus
  primer_bind (1)..(54)
gactcctcctcgggaaccggaaaggcgggccagcagcctgcaagaaaaagattg

SEQ ID NO: 96 - DNA, 39 bp, Adeno-Associated Virus
  primer_bind (1)..(39)
cagcagcgagtatcaaagacatctgcggataacaacaac

SEQ ID NO: 97 - DNA, 39 bp, Adeno-Associated Virus
  primer_bind (1)..(39)
tggactggagctaccaagtaccacctcaatggcagagac

SEQ ID NO: 98 - DNA, 42 bp, Adeno-Associated Virus
  primer_bind (1)..(42)
ccgggcccggccatggcaagccacaaggacgatgaagaaaag

SEQ ID NO: 99 - DNA, 45 bp, Adeno-Associated Virus
  primer_bind (1)..(45)
cacaaggacgatgaagaaaagttttttcctcagagcggggttctc

SEQ ID NO: 100 - DNA, 42 bp, Adeno-Associated Virus
  primer_bind (1)..(42)
gttctcatctttgggaagcaaggctcagagaaaacaaatgtg

SEQ ID NO: 101 - DNA, 30 bp, Adeno-Associated Virus
  primer_bind (1)..(30)
cctgggccctgcccacttacaacaaccatc

SEQ ID NO: 102 - DNA, 29 bp, Adeno-Associated Virus
  primer_bind (1)..(29)
cctgggtcttgcccacctacaacaaccac

SEQ ID NO: 103 - DNA, 36 bp, Adeno-Associated Virus
  primer_bind (1)..(36)
aacgcctactttggatacagcaccccctgggggtac

SEQ ID NO: 104 - DNA, 34 bp, Adeno-Associated Virus
  primer_bind (1)..(34)
cggggtcaaccgcgccagtgtcagcgccttcgcc

SEQ ID NO: 105 - DNA, 35 bp, Adeno-Associated Virus
  primer_bind (1)..(35)
gaaatatcaccagcttctcggacgtgcccgtcagc

SEQ ID NO: 106 - DNA, 38 bp, Adeno-Associated Virus
  primer_bind (1)..(38)
tcggacgtgcccgtcagcagcttcatcacccagtacag

SEQ ID NO: 107 - DNA, 35 bp, Adeno-Associated Virus
  primer_bind (1)..(35)
aagcgggcccaccgaaaccaaaacccaatcagcag

SEQ ID NO: 108 - DNA, 30 bp, Adeno-Associated Virus
  primer_bind (1)..(30)
aagctcgccgacgacacatccttcggggaa

SEQ ID NO: 109 - DNA, 30 bp, Adeno-Associated Virus
  primer_bind (1)..(30)
cgccaacaacctcacctccaccgtccaagt

SEQ ID NO: 110 - DNA, 29 bp, Adeno-Associated Virus
  primer_bind (1)..(29)
catgggccttgcccacctataacaaccac

SEQ ID NO: 111 - DNA, 27 bp, Adeno-Associated Virus
  primer_bind (1)..(27)
ctgggccctgcccacctacaacaacca

SEQ ID NO: 112 - DNA, 33 bp, Adeno-Associated Virus
  primer_bind (1)..(33)
gagaccggtagagccatcaccccagcgttctcc

SEQ ID NO: 113 - DNA, 35 bp, Adeno-Associated Virus
  primer_bind (1)..(35)
cccagcgttctccagactcctctacgggcatcggc

SEQ ID NO: 114 - DNA, 28 bp, Adeno-Associated Virus
  primer_bind (1)..(28)
ctacttcggctacagcaccccctggggg

SEQ ID NO: 115 - DNA, 40 bp, Adeno-Associated Virus
  primer_bind (1)..(40)
gacaaccgggcaaaacaacaatagcaactttgcctggact

SEQ ID NO: 116 - DNA, 42 bp, Adeno-Associated Virus
  primer_bind (1)..(42)
ccttcaaccagtcaaagctgaactctttcatcacgcaataca

SEQ ID NO: 117 - DNA, 35 bp, Adeno-Associated Virus
  primer_bind (1)..(35)
ttgaggaaggcgctaagacggctcctggaaagaag

SEQ ID NO: 118 - DNA, 42 bp, Adeno-Associated Virus; identical to SEQ ID NO: 85
  primer_bind (1)..(42)

SEQ ID NO: 119 - DNA, 40 bp, Adeno-Associated Virus
  primer_bind (1)..(40)
agatcctgatcaagaacacgcctgtacctgcggatcctcc

SEQ ID NO: 120 - DNA, 39 bp, Adeno-Associated Virus
  primer_bind (1)..(39)
aaagctgaactctttcatcacgcaatacagcaccggaca

SEQ ID NO: 121 - DNA, 30 bp, Adeno-Associated Virus
  primer_bind (1)..(30)
ggttgaggaaggcgctaagacggctcctgg

SEQ ID NO: 122 - DNA, 43 bp, Adeno-Associated Virus
  primer_bind (1)..(43)
aggcgctaagacggctcctggaaagaagagaccggtagagcca

SEQ ID NO: 123 - DNA, 56 bp, Adeno-Associated Virus
  primer_bind (1)..(56)
tggcctgaaacatcctccgcctcagatcctgatcaagaacacgcctgtacctgcgg

SEQ ID NO: 124 - DNA, 41 bp, Adeno-Associated Virus
  primer_bind (1)..(41)
caacgcctacttcggctacagcaccccctgggggtattttg

SEQ ID NO: 125 - DNA, 47 bp, Adeno-Associated Virus
  primer_bind (1)..(47)
cagctaccgacaacaacgtgtctcaaccactgtgactcaaaacaaca

SEQ ID NO: 126 - DNA, 54 bp, Adeno-Associated Virus
  primer_bind (1)..(54)
gccttcaacaaggacaagctgaactctttcatcacccagtattctactggccaa

SEQ ID NO: 127 - DNA, 49 bp, Adeno-Associated Virus
  primer_bind (1)..(49)
ccactgtgactcaaaacaacaacagcgaatttgcttggcctggagcttc

SEQ ID NO: 128 - DNA, 40 bp, Adeno-Associated Virus
  primer_bind (1)..(40)
cccgaacctgggccctgcccacctacaacaatcacctcta

SEQ ID NO: 129 - DNA, 43 bp, Adeno-Associated Virus
  primer_bind (1)..(43)
agcggctaagacggctcctggaaagaagaggcctgtagagcag

SEQ ID NO: 130 - DNA, 30 bp, Adeno-Associated Virus
  primer_bind (1)..(30)
gcctacgaccagcagctcaaagcgggtgac

SEQ ID NO: 131 - DNA, 29 bp, Adeno-Associated Virus
  primer_bind (1)..(29)
tggttgaggaaggcgctaagacggctcct

SEQ ID NO: 132 - DNA, 35 bp, Adeno-Associated Virus
  primer_bind (1)..(35)
gcagaatgaaggcaccaagaccatcgccaataacc

SEQ ID NO: 133 - DNA, 27 bp, Adeno-Associated Virus
  primer_bind (1)..(27)
gcagcaacgcgtctccacgacactgtc

SEQ ID NO: 134 - DNA, 38 bp, Adeno-Associated Virus
  primer_bind (1)..(38)
ctgtcgcaaaataacaacagcaactttgcctggaccgg

SEQ ID NO: 135 - DNA, 31 bp, Adeno-Associated Virus
  primer_bind (1)..(31)
caagctaagctggcgtcgttcatcacgcagt

SEQ ID NO: 136 - DNA, 31 bp, Adeno-Associated Virus
  primer_bind (1)..(31)
gcgtctgcaagaagatacgtcttttgggggc

SEQ ID NO: 137 - DNA, 27 bp, Adeno-Associated Virus
  primer_bind (1)..(27)
ctgggccctccccacctacaacaacca

SEQ ID NO: 138 - DNA, 29 bp, Adeno-Associated Virus
  primer_bind (1)..(29)
tggcgtcgttcatcacgcagtacagcacc

SEQ ID NO: 139 - DNA, 8344 bp, Adeno-Associated Virus
  gene (1)..(8344)
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagagtcctgtattagaggtcacgtgagtgttttgcgacattttgcgacaccatgtggtcacgctgggtatttaagcccgagtgagcacgcagggtctccattttgaagcgggaggtttgaacgcgcagcacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaaccaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaatttacgtgttaatcaataaaccggttgattcgtttcagttgaactttggtctcgcggccgctcgataagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcgcgagcggtatcagctccctcaaagcgtaataccggttatcccacagatcaggggatacgcagaaagacatgtgagtaaggcagccaaagtccaggaaccggtaaaaggccccgcgcgcttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctcacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagagtcctgtattagaggtcacgtgagtgttttgcgacattttgcgacaccatgtggtcacgctgggtatttaagcccgagtgagcacgcagggtctccattttgaagcgggaggtttgaacgcgcagcc

SEQ ID NO: 140 - DNA, 8267 bp, Adeno-Associated Virus
  gene (1)..(8267)
aattcccatcatcaataatataccttattttggattgaagccaatatgataatgagggggtggagtttgtgacgtggcgcggggcgtgggaacggggcgggtgacgtagtagtctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccaacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctccggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatgctctagagaaacctacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagttcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcctttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgatattttttttttcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctcccccgcgcgttggccgattcattaatgcag

SEQ ID NO: 141 - DNA, 8179 bp, Adeno-Associated Virus
  gene (1)..(8179)
aattcccatcatcaataatataccttatttaggattgaagccaatatgataatgagccggtggagtttgtgacgtggcgcggggcgtgggaacggggcgggtgacgtagtagtctctagagtcctgtattagaggtcacgtgagtgttttgcgactttttgcgacaccatgtggtcacgctgggtatttaagcccgagtgagcacgcagggtctccattttgaagcgggaggtttgaacgcgcagccaccacgggggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtgattgcttgttaatcaataaaccgtttaattcgtttcagttgaactttggtctctgcgtatttctttcttatctagtttccatgctctagactactacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 142 - DNA, 8173 bp, Adeno-Associated Virus
  gene (1)..(8173)
aattcccatcatcaataatataccttatttaggattgaagccaatatgataatgagccggtggagtttgtgacgtggcgcggggcgtgggaacggggcgggtgacgtagtagtctctagagtcctgtattagaggtcacgtgagtgttttgcgactttttgcgacaccatgtggtcacgctgggtatttaagcccgagtgagcacgcagggtctccattttgaagcgggaggtttgaacgcgcagccaccacgggggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaattgcttgttaatcaataaaccgtttaattcgtttcagttgaactttggtctctgcgtatttctttcttatctagtttccatgctctagactactacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 143 - DNA, 7226 bp, Adeno-Associated Virus
  gene (1)..(7226)
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaacccattcatgtcgcataccctcaataaaccgtgtattcgtgtcagtaaaatactgcctcttgtggtcatttctagaactagaactagtggatcccccgggctgcaggaattcgatatcaagcttatcgataccgtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 144 - DNA, 7226 bp, Adeno-Associated Virus; identical to SEQ ID NO: 143
  gene (1)..(7226)

SEQ ID NO: 145 - DNA, 8182 bp, Adeno-Associated Virus
  gene (1)..(8182)
aattcccatcatcaataatataccttatttaggattgaagccaatatgataatgagccggtggagtttgtgacgtggcgcggggcgtgggaacggggcgggtgacgtagtagtctctagagtcctgtattagaggtcacgtgagtgttttgcgactttttgcgacaccatgtggtcacgctgggtatttaagcccgagtgagcacgcagggtctccattttgaagcgggaggtttgaacgcgcagccaccacgggggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaattgcttgttaatcaataaaccgtttaattcgtttcagttgaactttggtctctgcgtatttctttcttatctagtttccatgctctagactactacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 146 - DNA, 8185 bp, Adeno-Associated Virus
  gene (1)..(8185)
aattcccatcatcaataatataccttatttaggattgaagccaatatgataatgagccggtggagtttgtgacgtggcgcggggcgtgggaacggggcgggtgacgtagtagtctctagagtcctgtattagaggtcacgtgagtgttttgcgactttttgcgacaccatgtggtcacgctgggtatttaagcccgagtgagcacgcagggtctccattttgaagcgggaggtttgaacgcgcagccaccacgggggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctggagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcttgttaatcaataaaccgtttaattcgtttcagttgaactttggtctctgcgtatttctttcttatctagtttccatgctctagactactacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 147 - DNA, 8261 bp, Adeno-Associated Virus
  gene (1)..(8261)
acggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgtttcggatctcaacggccttcacaggtacaagtgaacgcctttatcacccagtattctactggaaggtttttcctcgagtgggagctgcagaaggaaaaaagggcgctccgaacccggagatccagtacacttccaactattacaagtctaataatgttgaatttgttgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccaagaggtcacgctgggtatttaagcccgagtgagcacgcagggtctccattttgaagcgggaggtttgaacgcgcagccgccaagccgaattctgcagatatccatcacactggcggaaatggggaagtaaagagctgagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaaaagtccggcaactatggatgacgaaatagaacgatcgctgaaatagtgcctcactgatagcatggtaactgtcgaccagtttacctcatatatacttagatgattaactcatttatcaagattctgtaagtgaagaatccgcttttgatatctcatgacaattccttaacgtgatttcgtccaactgaacgctcgcatctcgtaagatcaggactctctgaatctttcagcgattcgcgttgcaccgtacagacgggtagtcagtgcatggcacacgttgcgcaactattactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagcccctcccgtatcgtagttatctacacgacgggggagtcagggcactatggatggaccaaatagacagatccccgaataggtgcctcctgattagcttggtaatggccgaccagttactctatatacttttattgggcgtctcacgtcagacccgtagaagatcaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagccccagcttggagcgaacgtcctaacaccgaatctgagattacctacagcgtgagttacacgtgacttgatagcgccacgcttccgagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctttttgatggccttgggctacgattccgactggaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggaaaccttataacaaaaagaagggcgggggtggcctcactgattaaaataacactttctcagacattcaggcgtaccgttcctgtctaaaatctctttaatcggcctcctgtttaactcccgctctgattctaacgaggaaacccttccgatttacgactggtagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcgattttttaggatcgatttagtgctttacggcacctcgaccccaaaaacttgattagggtgatggttcacgtattgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtgagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttactacacgacggggagtcaggcaactatggatgaacgaaatagacagatcgctgagataggtgcctcactgattaagcattgg

SEQ ID NO: 148 - DNA, 7775 bp, Adeno-Associated Virus
  gene (1)..(7775)
ggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaacgggtggtggcgcagcatctgacgcacgtgtcgcagacgcaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccatttctcggaggaaagcaaaggtgcgcgtggaccagaaatgcaagtcctctcactcacggacagaaagactgtttagagtgctttcccgtgtcagaatctcaacccgtttctgtcgtcaaaaaggcgtatcaaaaaactgtgctacattcatcatatcatgggaaaggtgccagacgcttgcactgcctgcgatctggtcaatgtggatttggatgactgcatctttgaacaataaatgatttaaatcaggtatggctgccgatggttatcgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctcaatgtggatttggatgactgcatctttgaacaatatatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcagcccctctggtctgggatctggtacaatggctgcaggcggtggcgctccaatggcagacaatccccaaccccaattttcagaacctccggaaaaaaaatcctcttttgggggccacccccggccaaccatccttccagcccaaaaaccggttccccaaccctttcgtcttggttgagaaaggccctaaaacgctcccggaaaaaaaaaaaccggaaaaccctcccccccaccttcccccaaatcctccaacggcctccgcaaaaaaaggccaccaccccccaaaaaaaaaatccactttgggcaaaacggccaatccaaatccgtggcccaacctccacccatccgaaaacccccccccagcccctctgggccgggatttggtaacatggctggcggggggggcccccccatgggaaccctccccacctacaacaaccaccaaaataaacaaagcccccaacggaggggttattcccccagaaattggggaagcaccaacgacaacacctacttcggctacagcaccccctgggggtattttgactttaacagattccactgccacttctcaccacgtgactggcagcgactcatcaacaacaactggggattccggcccaaaaaactcaacttcaagctcttcaacatccacgtcaaggaggtcacgcaaaatgaaggcaccaagaccatcgccaataaccttaccagcacgaatcaggtctttaccgactcggaataccagctcccgtacctcctcggctctgcgcaccagggctgcctgcctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagaaactctctggtaaatcccggtgtcgctatggcaacccacaaggacgacgaagagcgattttttccgtccagcggagtcttaatgtttgggaaacagggagctggaaaagacaacgtggactatagcatcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctgtcatggtctggcagaaccgggacgtgtacctgcagggtcctatctgggccaagattcctcacacggacggaaactttcatccctcgccgctgatgggaggctttggactgaaacacccgcctcctccatcctgattaaaaatacacctgttcccgtggatcctccaactaccttcagtccagctaagctggcgtcgttcatcacgcagtacagcaccggacaggtcagcgtggaaattgaatgggagctgcaaaaaaaaaacagcaaacgctggaacccaaaaattcaatactcttccaactactacaaatctacaaatgtggactttgctgttaacacagatggcacttattggtacgatcagactggagcgggcagtgagcgcacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggtgctttacggcacctcgaccccaaaaaacttgattagggtgatggttcacgaaaagggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgcccgggcaagagcaactcggtcgccgcatacactattctcagatgacttggttgagtactccccagtcacagaaaagcatcttacgatggcatgacagtaaggagattatgcatgctgccataccatgatgaataacactgcgcacttacttctgacacgatcggaggaccgaaggagctcggcgcttctggggctttttttgaaggctaaaaaagggcccaaggggggggggggggggcaagcacaaccccccgtacgtcgggggggggagaaggagattttcctttagcaaagagaacggtgccccccccggcgagggaacggggcccctggtttcccccgcccaacggagaaaccatacacccactactatgcacaacatctatatgtaatcataaaaccatagatcatcgctatagcgcgacgcgcccctctctgcatactgtgaggtaagacgatctgtggctccgattctcgacatattgacgctagactgcgttgctcagtcttgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcagggggggcggagcctatggaaaaacgccagcaacgcggcctttttacggttgaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctcccgcaaagatacagactggagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtg

SEQ ID NO: 149 - DNA, 8344 bp, Adeno-Associated Virus
  gene (1)..(8344)
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagagtcctgtattagaggtcacgtgagtgttttgcgacattttgcgacaccatgtggtcacgctgggtatttaagcccgagtgagcacgcagggtctccattttgaagcgggaggtttgaacgcgcagcacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaaccaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctagaacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccgcctacaataaccacctctacaagcaaatctccagtgcttcaacgggggccagcaacgacaaccactacttcggctacgcccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacgccaattttacctggactggtgcttcaaaatataacctcaatgggcgtgaatccatcatcaaccctggcactgctatggccgcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagtttgcagctacaaagtttgctgcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaatttacgtgttaatcaataaaccggttgattcgtttcagttgaactttggtctcgcggccgctcgataagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcgcgagcggtatcagctccctcaaagcgtaataccggttatcccacagatcaggggatacgcagaaagacatgtgagtaaggcagccaaagtccaggaaccggtaaaaggccccgcgcgcttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctcacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagagtcctgtattagaggtcacgtgagtgttttgcgacattttgcgacaccatgtggtcacgctgggtatttaagcccgagtgagcacgcagggtctccattttgaagcgggaggtttgaacgcgcagcc

SEQ ID NO: 150 - DNA, 8267 bp, Adeno-Associated Virus
  gene (1)..(8267)
aattcccatcatcaataatataccttattttggattgaagccaatatgataatgagggggtggagtttgtgacgtggcgcggggcgtgggaacggggcgggtgacgtagtagtctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccaacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagaacggcatagggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaggacggccccgggaaaaaggaggccggtagagcactctcctgtggagccagactcctcctcgggaaccggaagggccggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctccggaaattggcattgcgattccacatggatgggcgacagagtcatcaccaccagcacccgaacctgggccctgccggcctacaacaaccacctctacaaacaaatttccagccaatcaggagcctcgaacgacaatcactactttggctacgccaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttaagcagaacaaacactccaagtggagccaccacgcagtcaaggcttcagttttctcaggccggagcgagtgacattcgggaccagtctaggaactggcttcctggaccctgttaccgccagcagcgagtagctaagacatctgcagataacaacaacgctgaatactcgtgggctggagctaccagataccacctcaatggcagagactctctggtgaatccgggcccggcgatggcagcccacagggacgatgaagaaaggttttttcctcaggccggggttctcatctttggtagacaaggcgccgagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccgtcgaccaccttcagtgcggccaagtttgctgccttcatcgcacagtactccacgggacaggtcagcgtggagatcgagtgggagctgcagaaggaaaacagcaaacgctggaatcccgaaattcagtacgcttccaactacaacaagtctgttaatgtagactttgctgtggacgctaatggcgtgtattcagagcctcgccccattggcaccagatacctgactcgtaatctgtaattgcttgttaatcaataaaccgtttaattcgtttcagttgaactttggtctctgcgtatttctttcttatctagtttccatgctctagagaaacctacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagttcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcctttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgatattttttttttcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctcccccgcgcgttggccgattcattaatgcag

SEQ ID NO: 151 - DNA, 8179 bp, Adeno-Associated Virus
  gene (1)..(8179)
aattcccatcatcaataatataccttatttaggattgaagccaatatgataatgagccggtggagtttgtgacgtggcgcggggcgtgggaacggggcgggtgacgtagtagtctctagagtcctgtattagaggtcacgtgagtgttttgcgactttttgcgacaccatgtggtcacgctgggtatttaagcccgagtgagcacgcagggtctccattttgaagcgggaggtttgaacgcgcagccaccacgggggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctagaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccgcttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagacttgcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgctgcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtgattgcttgttaatcaataaaccgtttaattcgtttcagttgaactttggtctctgcgtatttctttcttatctagtttccatgctctagactactacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 152 - DNA, 8172 bp, Adeno-Associated Virus
  gene (1)..(8172)
aattcccatcatcaataatataccttatttaggattgaagccaatatgataatgagccggtggagtttgtgacgtggcgcggggcgtgggaacggggcgggtgacgtagtagtctctagagtcctgtattagaggtcacgtgagtgttttgcgactttttgcgacaccatgtggtcacgctgggtatttaagcccgagtgagcacgcagggtctccattttgaagcgggaggtttgaacgcgcagccaccacgggggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaggagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccgcctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttcgcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaacgccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaattgcttgttaatcaataaaccgtttaattcgtttcagttgaactttggtctctgcgtatttctttcttatctagtttccatgctctagactactacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgca

SEQ ID NO: 153 - DNA, 7226 bp, Adeno-Associated Virus
  gene (1)..(7226)
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatagtcatgtcttttgttgatcaccctccagattggttggaagaagttggtgaaggtcttcgcgagtttttgggccttgaagcgggcccaccgaaaccagaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacgcatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacgccaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcgcctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccgctgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttcgcggacgtgcccgtcagcgccttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaacccattcatgtcgcataccctcaataaaccgtgtattcgtgtcagtaaaatactgcctcttgtggtcatttctagaactagaactagtggatcccccgggctgcaggaattcgatatcaagcttatcgataccgtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 154 - DNA, 8179 bp, Adeno-Associated Virus
  gene (1)..(8179)
aattcccatcatcaataatataccttatttaggattgaagccaatatgataatgagccggtggagtttgtgacgtggcgcggggcgtgggaacggggcgggtgacgtagtagtctctagagtcctgtattagaggtcacgtgagtgttttgcgactttttgcgacaccatgtggtcacgctgggtatttaagcccgagtgagcacgcagggtctccattttgaagcgggaggtttgaacgcgcagccaccacgggggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaggacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccgcctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgttgctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgctgcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgcttgttaatcaataaaccgtttaattcgtttcagttgaactttggtctctgcgtatttctttcttatctagtttccatgctctagactactacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 155 - DNA, 8182 bp, Adeno-Associated Virus
  gene (1)..(8182)
aattcccatcatcaataatataccttatttaggattgaagccaatatgataatgagccggtggagtttgtgacgtggcgcggggcgtgggaacggggcgggtgacgtagtagtctctagagtcctgtattagaggtcacgtgagtgttttgcgactttttgcgacaccatgtggtcacgctgggtatttaagcccgagtgagcacgcagggtctccattttgaagcgggaggtttgaacgcgcagccaccacgggggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaggacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccgcctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtcgccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgctgcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaattgcttgttaatcaataaaccgtttaattcgtttcagttgaactttggtctctgcgtatttctttcttatctagtttccatgctctagactactacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 156 - DNA, 8185 bp, Adeno-Associated Virus
  gene (1)..(8185)
aattcccatcatcaataatataccttatttaggattgaagccaatatgataatgagccggtggagtttgtgacgtggcgcggggcgtgggaacggggcgggtgacgtagtagtctctagagtcctgtattagaggtcacgtgagtgttttgcgactttttgcgacaccatgtggtcacgctgggtatttaagcccgagtgagcacgcagggtctccattttgaagcgggaggtttgaacgcgcagccaccacgggggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctggagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggggctagggcggctcctggaaggagaagaccggtagagccagcaccccagcgtgctccagacgcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgccggcctacaacaaccacctctacaagcaaatctccaacgggacatcgggaggagccaccaacgacaacacctacttcggctacgccaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatgccaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatccgaaacgcgcctgtacctgcggatcctccggctaccttcaaccaggcaaagctgaacgctttcatcgcgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcttgttaatcaataaaccgtttaattcgtttcagttgaactttggtctctgcgtatttctttcttatctagtttccatgctctagactactacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 157 - DNA, 8262 bp, Adeno-Associated Virus
  gene (1)..(8262)
acggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagagaaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccgcctacaacaatcacctctacaagcaaatctccaacagcacatctggaggatcttcaaatgacaacgcctacttcggctacgcaaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtcgcgaccactgtgactcaaaacaacaacgccgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaacgccttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgtttcggatctcaacggccttcacaggtacaagtgaacgcctttatcacccagtattctactggaaggtttttcctcgagtgggagctgcagaaggaaaaaagggcgctccgaacccggagatccagtacacttccaactattacaagtctaataatgttgaatttgttgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccaagaggtcacgctgggtatttaagcccgagtgagcacgcagggtctccattttgaagcgggaggtttgaacgcgcagccgccaagccgaattctgcagatatccatcacactggcggaaatggggaagtaaagagctgagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaaaagtccggcaactatggatgacgaaatagaacgatcgctgaaatagtgcctcactgatagcatggtaactgtcgaccagtttacctcatatatacttagatgattaactcatttatcaagattctgtaagtgaagaatccgcttttgatatctcatgacaattccttaacgtgatttcgtccaactgaacgctcgcatctcgtaagatcaggactctctgaatctttcagcgattcgcgttgcaccgtacagacgggtagtcagtgcatggcacacgttgcgcaactattactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagcccctcccgtatcgtagttatctacacgacgggggagtcagggcactatggatggaccaaatagacagatccccgaataggtgcctcctgattagcttggtaatggccgaccagttactctatatacttttattgggcgtctcacgtcagacccgtagaagatcaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagccccagcttggagcgaacgtcctaacaccgaatctgagattacctacagcgtgagttacacgtgacttgatagcgccacgcttccgagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctttttgatggccttgggctacgattccgactggaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggaaaccttataacaaaaagaagggcgggggtggcctcactgattaaaataacactttctcagacattcaggcgtaccgttcctgtctaaaatctctttaatcggcctcctgtttaactcccgctctgattctaacgaggaaacccttccgatttacgactggtagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcgattttttaggatcgatttagtgctttacggcacctcgaccccaaaaacttgattagggtgatggttcacgtattgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtgagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgg

SEQ ID NO: 158 - DNA, 7716 bp, Adeno-Associated Virus
  gene (1)..(7716)
ggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaacgggtggtggcgcagcatctgacgcacgtgtcgcagacgcaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccatttctcggaggaaagcaaaggtgcgcgtggaccagaaatgcaagtcctctcactcacggacagaaagactgtttagagtgctttcccgtgtcagaatctcaacccgtttctgtcgtcaaaaaggcgtatcaaaaaactgtgctacattcatcatatcatgggaaaggtgccagacgcttgcactgcctgcgatctggtcaatgtggatttggatgactgcatctttgaacaataaatgatttaaatcaggtatggctgccgatggttatcgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctcaatgtggatttggatgactgcatctttgaacaatatatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcagagcgggtgacaatccgtacctgcggtataaccacgccgacgccgagtttcaggagcgtctgcaagaagatgcgtctttttctggttgaggaaggcgctaggacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcagcccctctggtctgggatctggtacaatggctgcaggcggtggcgctccaatggcagacaatccccaaccccaattttcagaacctccggaaaaaaaatcctcttttgggggccacccccggccaaccatccttccagcccaaaaaccggttccccaaccctttcgtcttggttgagaaaggccctaaaacgctcccggaaaaaaaaaaaccggaaaaccctcccccccaccttcccccaaatcctccaacggcctccgcaaaaaaaggccaccaccccccaaaaaaaaaatccactttgggcaaaacggccaatccaaatccgtggcccaacctccacccatccgaaaacccccccccagcccctctgggccgggatttggtaacatggctggcggggggggcccccccatgggaaccctccccgcctacaacaaccaccaaaataaacaaagcccccaacggaggggttattcccccagaaattggggaagcaccaacgacaacacctacttcggctacagcaccccctgggggtattttgactttaacagattccactgccacttctcaccacgtgactggcagcgactcatcaacaacaactggggattccggcccaaaaaactcaacttcaagctcttcaacatccacgtcaaggaggtcacgcaaaatgaaggcaccaggaccatcgccaataaccttaccagcacgaatcaggtctttaccgactcggaataccagctcccgtacctcctcggctctgcgcaccagggctgcctgcctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtcgccacgacactgtcgcaaaataacaacgccaactttgcctggaccggtgccaccaagtatcatctgaatggcagaaactctctggtaaatcccggtgtcgctatggcaacccacaaggacgacgaagagcgattttttccgtccagcggagtcttaatgtttgggaaacagggagctggaaaagacaacgtggactatagcatcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctgtcatggtctggcagaaccgggacgtgtacctgcagggtcctatctgggccaagattcctcacacggacggaaactttcatccctcgccgctgatgggaggctttggactgaaacacccgcctcctccatcctgattaaaaatacacctgttcccgtggatcctccaactaccttcagtccagctaagctggcggcgttcatcgcgcagtacagcaccggacaggtcagcgtggaaattgaatgggagctgcaaaaaaaaaacagcaaacgctggaacccaaaaattcaatactcttccaactactacaaatctacaaatgtggactttgctgttaacacagatggcacttattggtacgatcagactggagcgggcagtgagcgcacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggtgctttacggcacctcgaccccaaaaaacttgattagggtgatggttcacgaaaagggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgcccgggcaagagcaactcggtcgccgcatacactattctcagatgacttggttgagtactccccagtcacagaaaagcatcttacgatggcatgacagtaaggagattatgcatgctgccataccatgatgaataacactgcgcacttacttctgacacgatcggaggaccgaaggagctcggcgcttctggggctttttttgaaggctaaaaaagggcccaaggggggggggggggggcaagcacaaccccccgtacgtcgggggggggagaaggagattttcctttagcaaagagaacggtgccccccccggcgagggaacggggcccctggtttcccccgcccaacggagaaaccatacacccactactatgcacaacatctatatgtaatcataaaaccatagatcatcgctatagcgcgacgcgcccctctctgcatactgtgaggtaagacgatctgtggctccgattctcgacatattgacgctagactgcgttgctcagtcttgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcagggggggcggagcctatggaaaaacgccagcaacgcggcctttttacggttgaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctcccgcaaagatacagactggagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtg

SEQ ID NO: 159 - DNA, 19 bp, Mus musculus
  primer_bind (1)..(19)
tttccacagccacgaagct

SEQ ID NO: 160 - DNA, 20 bp, Mus musculus
  primer_bind (1)..(20)
tgatgaaagggctcccaagt

SEQ ID NO: 161 - DNA, 21 bp, Mus musculus
  primer_bind (1)..(21)
tgttctctgggaaatcgtgga

SEQ ID NO: 162 - DNA, 21 bp, Mus musculus
  primer_bind (1)..(21)
tcagaattgccattcgacaac

SEQ ID NO: 163 - DNA, 17 bp, Mus musculus
  primer_bind (1)..(17)
ccagaactccaggcggt

SEQ ID NO: 164 - DNA, 21 bp, Mus musculus
  primer_bind (1)..(21)
tgggaacttctcatccctttg

SEQ ID NO: 165 - DNA, 19 bp, Mus musculus
  primer_bind (1)..(19)
gcctatcgccaatgagctg

SEQ ID NO: 166 - DNA, 18 bp, Mus musculus
  primer_bind (1)..(18)
ttcggtttgggtgcagtg

SEQ ID NO: 167 - DNA, 20 bp, Mus musculus
  primer_bind (1)..(20)
ctttgcctacctctccctcg

SEQ ID NO: 168 - DNA, 19 bp, Mus musculus
  primer_bind (1)..(19)
cacacttggcggttccttc

SEQ ID NO: 169 - DNA, 20 bp, Mus musculus
  primer_bind (1)..(20)
tgccacctactcccttggat

SEQ ID NO: 170 - DNA, 18 bp, Mus musculus
  primer_bind (1)..(18)
ggctgttggaacgctgac

SEQ ID NO: 171 - DNA, 22 bp, Mus musculus
  primer_bind (1)..(22)
cagcttaaagggcgggtcagag

SEQ ID NO: 172 - DNA, 22 bp, Mus musculus
  primer_bind (1)..(22)
tggagacgccagctctggctca

SEQ ID NO: 173 - DNA, 20 bp, Mus musculus
  primer_bind (1)..(20)
ccagacgctcttcgagaacc

SEQ ID NO: 174 - DNA, 20 bp, Mus musculus
  primer_bind (1)..(20)
gttatagaagtggcggttgt

(19) United States
(12) Patent Application Publication
(10) Pub. No.: US 2013/0295614 A1
(43) Pub. Date: Nov. 7, 2013

(54) NUCLEOTIDE SEQUENCES, METHODS, KIT AND A RECOMBINANT CELL THEREOF

(71) Applicants: Sangeetha Hareendran, Vellore (IN); Nishanth Gabriel, Vellore (IN); Dwaipayan Sen, Vellore (IN); Rupali Gadkar, Bangalore (IN); Sudha Govindarajan, Bangalore (IN); Narayana Swamy Srinivasan, Bangalore (IN); Arun Srivastava, Gainesville, FL (US); Alok Srivastava, Vellore (IN); Giridhara Rao Jayandharan, Vellore (IN); Ruchita Selot, Vellore (IN); Balaji Balakrishnan, Vellore (IN); Akshaya Krishnagopal, Vellore (IN)

(72) Inventors: Sangeetha Hareendran, Vellore (IN); Nishanth Gabriel, Vellore (IN); Dwaipayan Sen, Vellore (IN); Rupali Gadkar, Bangalore (IN); Sudha Govindarajan, Bangalore (IN); Narayana Swamy Srinivasan, Bangalore (IN); Arun Srivastava, Gainesville, FL (US); Alok Srivastava, Vellore (IN); Giridhara Rao Jayandharan, Vellore (IN); Ruchita Selot, Vellore (IN); Balaji Balakrishnan, Vellore (IN); Akshaya Krishnagopal, Vellore (IN)

(21) Appl. No.: 13/886,241

(22) Filed: May 2, 2013

(30) Foreign Application Priority Data
May 2, 2012 (IN) 1714/CHE/2012
Jun. 4, 2012 (IN) 2231/CHE/2012

(51) Int. Cl. C12N 15/86; C12N 15/10

(52) U.S. Cl. CPC C12N 15/86; C12N 15/102
USPC 435/91.4; 536/23.72; 435/456; 435/325

(57) ABSTRACT

The present disclosure relates to recombinant adeno-associated virus (AAV) vector serotype, wherein the capsid protein of AAV serotypes is mutated at single or multiple sites. The disclosure further relates to an improved transduction efficiency of these mutant AAV serotypes. The AAV serotypes disclosed are AAV1, AAV2, AAV3, AAV4, AAV5, AAV6, AAV7, AAV8, AAV9, AAV10. The instant disclosure relates to nucleotide sequences, recombinant vector, methods and kit thereof.

### CROSS-REFERENCE TO RELATED APPLICATION

This application claims priority to Indian Patent Application No. 1714/CHE/2012, filed on May 2, 2012 in the Indian Intellectual Property Office, and Indian Patent Application No. 2231/CHE/2012, filed on Jun. 4, 2012 in the Indian Intellectual Property Office, the disclosures of which are incorporated herein by reference in their entireties.

### TECHNICAL FIELD

The present disclosure relates to recombinant adeno-associated virus (AAV) vector serotype, wherein the capsid protein of AAV serotypes is mutated at single or multiple sites. The disclosure further relates to an improved transduction efficiency of these mutant AAV serotypes. The AAV serotypes disclosed are AAV1, AAV2, AAV3, AAV4, AAV5, AAV6, AAV7, AAV8, AAV9, AAV10. The instant disclosure thus relates to nucleotide sequences, recombinant vector, methods and kit thereof.

### BACKGROUND OF THE DISCLOSURE

Intracellular trafficking of virus from cytoplasm to nucleus is one of the crucial rate limiting events in determining the efficiency of gene transfer with AAV. However, their therapeutic efficiency when targeted to organ systems, such as during hepatic gene transfer in patients with hemophilia B, is suboptimal because of the CD8+ T cell response directed against the AAV capsid particularly at higher administered vector doses (≧2×10¹² viral genomes [VG]/kg) (Manno et al., 2006). A similar theme of vector dose-dependent immunotoxicity has emerged from the use of alternative AAV serotypes in other clinical trials as well (Stroes et al., 2008). More recently, in the recombinant AAV8-mediated gene transfer for hemophilia B (Nathwani et al., 2011), two patients who received the highest dose (2×10¹² VG/kg) of vector required glucocorticoid therapy to attenuate a capsid-specific T cell response developed against capsid. Further, a majority of AAV vectors are phosphorylated and degraded in the cytoplasm. The use of proteasomal inhibitors is known to result in a ˜2 fold increase in gene expression from AAV serotypes (Monahan et al., 2010). However, systemic administration of these proteasomal inhibitors leads to severe side effects (Rajkumar et al., 2005). Therefore, irrespective of whether an alternative AAV serotype or an immune suppression protocol is used, it is important to develop novel AAV vectors that provide enhanced gene expression at significantly lower vector doses to achieve successful gene transfer in humans.

### STATEMENT OF THE DISCLOSURE

Accordingly, the present disclosure relates to a nucleotide sequence selected from a group comprising SEQ ID Nos. 139 to 148, having mutation at codon selected from a group comprising TCT, TCC, TCA, TCG, AGT, AGC, ACT, ACC, ACA, ACG, AAA and AAG or any combination thereof; a nucleotide sequence selected from a group comprising SEQ ID Nos. 70 to 138, having mutation at codon selected from a group comprising TCT, TCC, TCA, TCG, AGT, AGC, ACT, ACC, ACA, ACG, AAA and AAG or any combination thereof; a method of obtaining a nucleotide sequence selected from a group comprising SEQ ID Nos. 139 to 148, having mutation at codon selected from a group comprising TCT, TCC, TCA, TCG, AGT, AGC, ACT, ACC, ACA, ACG, AAA and AAG or any combination thereof, said method comprising act of introducing mutation in any of nucleotide sequence selected from a group comprising SEQ ID Nos. 139 to 148 through site directed mutagenesis by using the nucleotide sequence as mentioned above; a method of enhancing transduction efficiency, said method comprising act of expressing a target gene in presence of a nucleotide sequence selected from a group comprising SEQ ID Nos. 139 to 148, having mutation at codon selected from a group comprising TCT, TCC, TCA, TCG, AGT, AGC, ACT, ACC, ACA, ACG, AAA and AAG or any combination thereof; a recombinant cell comprising the nucleotide sequence as mentioned above; a method of obtaining the recombinant cell as mentioned above, said method comprising act of introducing the nucleotide sequence as mentioned above to a host cell, to obtain said recombinant cell; and a kit comprising a nucleotide sequence selected from a group comprising SEQ ID Nos. 70 to 138, having mutation at codon selected from a group comprising TCT, TCC, TCA, TCG, AGT, AGC, ACT, ACC, ACA, ACG, AAA and AAG or any combination thereof.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figure together with a detailed description below, are incorporated in and form part of the specification, and serve to further illustrate the embodiments and explain various principles and advantages, in accordance with the present disclosure wherein:

FIG. 1: illustrates increased transduction efficiency in vitro and in vivo of AAV1 serine/lysine mutant vectors. (a) In vitro transduction efficiency of AAV 1 vectors. Equal number of CHO cells is mock-infected or infected with 5×10³ vgs/cell of the different AAV1 vectors. Forty eight hours later, the luciferase activity in the cell lysate is measured using a commercial kit (BioVision Inc, Milpitas, Calif., USA) in a GlowMax™ 20/20 luminometer (Promega, WI, USA). The data depicted are mean of triplicate analysis. Fold increase in transduction in comparison to WT-AAV1 infected cells are shown. *p<0.05 Vs WT-AAV1 infected cells. (b) In vivo bio-luminescence imaging of AAV1 vector administered mice. Animals injected with 5×10¹⁰ vgs of AAV1-Luciferase vectors are imaged 2-weeks after gene transfer in an IVIS Spect-CT small animal imaging system (Perkin Elmer, Caliper Life Sciences). (c) The fold change in luciferase expression from animals injected with AAV1-K137R or AAV1-S669A vectors are shown in comparison to WT-AAV1 injected mice. *p<0.05 Vs WT-AAV1 injected mice. (d) Approximately 3×10⁸ viral particles of WT-AAV1 and K137R-AAV1 vectors are denatured at 95° C. for 5 minutes. The denatured viral particles are then used to perform the ubiquitin conjugation assay according to the manufacturer's protocol. The processed samples are electrophoresed on a 4-20% denaturing polyacrylamide gel and the ubiquitination pattern detected by immunoblotting. The mono-to-polyubiquitin conjugates are detected as a smear at molecular weight >150 Kda. (e) VP1 (87 Kda), VP2 (72 KDa) and VP3 (62 Kda) capsid proteins are immunoblotted as loading controls.

FIG. 2: illustrates the schematic representation of the serine (S), threonine (T) and lysine (K) residues mutated in AAV1 and their conservation status across other AAV serotypes. VP1 protein sequences from AAV serotypes 1 through 10 are aligned by ClustalW and the conservation status of the each of the target site for mutagenesis is shown in red.

FIG. 3: illustrates the Structural analysis of phosphodegrons 1-3 in AAV2 capsid. In particular FIGS. 1a, c and e show phosphodegrons 1, 2 and 3 colored in green, respectively and the corresponding zoomed in regions of the three phosphodegrons are shown in b, d and f respectively. Phosphodegrons in the AAV2 capsid are largely present in the loop regions and are solvent exposed as shown in the figure. The phosphorylation and ubiquitination sites in the phosphodegrons are shown as green and blue spheres respectively. Receptor binding residues that have been also predicted as ubiquitination sites are shown as purple spheres. The acidic residues in phosphodegron 1 and 3 and prolines in phosphodegron 2 are colored red while rest of the protein structure is shown in grey. The images are generated using PYMOL software (DeLlano, 2002)

FIG. 4: illustrates the Schematic representation and conservation status of the various serine (S), threonine (T) and lysine (K) residues mutated in AAV2 capsid. VP1 protein sequences from AAV serotypes 1 through 10 are aligned by ClustalW and the conservation status of the each of the mutated site is given. S/T residues are shown in panel A while lysine residues in panel B. S/T/K residues within phosphodegrons 1, 2 and 3 are shown in red font while those chosen based on evolutionary conservation is shown in green font. Those residues which are chosen based on either in silico prediction to be a part of a phosphosite or high ubiquitination score on UbPred tool are shown in blue. A control threonine mutation shown in brown font is chosen as a negative control for the mutation experiments.

FIG. 5: illustrates the effect of pharmacological inhibition of host cellular serine/threonine kinases on AAV2 mediated gene expression. (A) HeLa cells are mock-(PBS) treated or pre-treated with the protein kinase A (PKA), protein kinase C (PKC), casein kinase (CK)-II inhibitors (i) either alone or in combinations shown, 24 hrs prior to transduction with AAV2-EGFP vectors. Twenty four hours post-transduction, cell suspensions are analyzed for EGFP expression by flow cytometry (B) Quantitative representation of the data from (A). One way analysis of variance (ANOVA) test is used for statistical analysis. *p<0.05; **p<0.01 Vs AAV2-WT infected cells.

FIG. 6: illustrates the increased transduction efficiency of AAV2 serine/threonine/lysine mutant vectors in vitro. HeLa cells are either mock-infected or infected with 2×10³ vgs/cell of AAV2-WT or AAV2-S/T→A (A) or AAV2-K→R (C) mutant vectors and cells analyzed for EGFP expression 48 hrs later by flow cytometry. The percentage EGFP positive cells post-transduction with either serine/threonine mutants (B) or lysine mutants (D) are shown. Similar experiments are carried out in HEK 293 cells with an MOI of 2×10³ vgs/cell of AAV2-WT or AAV2 S/T/K mutant vectors (E). Quantitative analysis of this data by flow cytometric analysis is shown in F. The data depicted in A, C, and E are representative histograms while the data in B, D, F are mean of triplicate analysis. One way analysis of variance (ANOVA) test is used for statistical analysis.*p<0.05, **p<0.01 Vs AAV2-WT infected cells.

FIG. 7: illustrates the fluorescence imaging of HeLa cells infected with AAV2 wild-type or S/T/K mutant vectors. HeLa cells are either mock-infected or infected with 2×10³ vgs/cell of AAV2-WT or AAV2-S/T/K mutant vectors. Forty eight hours later, the cells are analyzed by fluorescence microscopy (A) Visual comparison of AAV2 S/T→A mutants compared to AAV2-WT vectors. (B) Visual comparison of AAV2 K→R mutants compared to AAV2-WT vectors.

FIG. 8: illustrates enhanced transduction upon hepatic gene transfer of AAV2 serine/threonine mutant vectors in vivo. (A) Transgene expression is detected by fluorescence microscopy 4-weeks post-injection with 5×10¹⁰ vector particles/animal of scAAV2-EGFP, control scAAV8-EGFP or AAV2 mutant S/T vectors. Representative images of hepatic tissues from four different animals in each group are shown. (B) Estimation of vector genome copies in the liver after AAV mediated gene transfer. Genomic DNA is isolated from liver tissue of C57BL/6 mice 4-weeks post vector administration and the viral copy numbers estimated by a quantitative PCR (C) Analysis of EGFP transcript levels by real-time quantitative PCR. Hepatic RNA isolated from animals injected with AAV2-WT, AAV8-WT or AAV2 S/T vectors are analyzed for EGFP expression and the data normalized to the GAPDH reference gene. One way analysis of variance (ANOVA) test is used for the statistical comparisons. *p<0.05 Vs AAV2-WT injected animals.

FIG. 9: illustrates the analysis of AAV2 lysine mutant vector-mediated EGFP expression in hepatocytes of normal C57BL/6 mice in comparison to wild-type AAV2 vectors. (A) Transgene expression is detected by fluorescence microscopy 4-weeks post-injection of 5×10¹⁰ vector particles/animal of scAAV2-EGFP or AAV2 K→R mutant vectors. Representative images of hepatic tissues from four different animals in each group are shown. (B) Estimation of vector genome copies in the liver after AAV mediated gene transfer. Genomic DNA is isolated from liver tissue of C57BL/6 mice 4-weeks post vector administration and the viral copy numbers estimated by a quantitative PCR as described in the “materials and methods”. (C) Analysis of EGFP transcript levels by real-time quantitative PCR. Hepatic RNA isolated from animals injected with AAV2-WT or K→R mutant vectors are analyzed for EGFP expression and the data normalized to the GAPDH reference gene. One way analysis of variance (ANOVA) test is used for the statistical comparisons. *p<0.05 Vs AAV2-WT injected animals.

FIG. 10: illustrates reduced ubiquitination of AAV2 lysine mutant K532R in comparison to AAV2-WT or AAV5-WT vectors. (A) Approximately 3×10⁸ viral particles of AAV2-WT, AAV5-WT and AAV2 K532R vectors are denatured at 95° C. for 5 minutes. The denatured viral particles are then used to perform the ubiquitin conjugation assay. The processed samples are electrophoresed on a 5-20% denaturing polyacrylamide gel and the ubiquitination pattern detected by immunoblotting using an anti-ubiquitin antibody. The mono-to-polyubiquitin conjugates are detected as a smear at molecular weight >150 Kda. (B) AAV Capsid VP1, VP2 and VP3 proteins are used as loading control.

FIG. 11: illustrates the histological examination of C57BL/6 liver samples 4-weeks post-injection of AAV2-WT or mutant vectors. Hepatic sections are fixed in 10% buffered formalin and stained with hematoxylin-eosin. The median inflammation score (IS) for each group is indicated below the images (Magnification—40×) with the range of values given within brackets. Arrowhead and thin arrow denote portal and focal lobular inflammation, respectively. Representative image of one animal liver from each group (n=3) is shown.

FIG. 12: illustrates enhanced transduction upon hepatic gene transfer of AAV2 serine/threonine/Lysine multiple-mutant vectors in vivo. (A) Transgene expression is detected by fluorescence microscopy 4-weeks post-injection with 5×10¹⁰ vector particles/animal of scAAV2-EGFP or multiple AAV2 mutant S/T/K vectors. Representative images of hepatic tissues from four different animals in each group are shown. (B) Analysis of EGFP transcript levels by real-time quantitative PCR. Hepatic RNA isolated from animals injected with AAV2-WT or AAV2 S/T/K vectors are analyzed for EGFP expression and the data normalized to the GAPDH reference gene. (C) Estimation of vector genome copies in the liver after AAV mediated gene transfer. Genomic DNA is isolated from liver tissue of C57BL/6 mice 4-weeks post vector administration and the viral copy numbers estimated by a quantitative PCR. One way analysis of variance (ANOVA) test is used for the statistical comparisons. *p<0.05 Vs AAV2-WT injected animals.

FIG. 13: illustrates reduced cross-neutralizing antibody formation for AAV2 triple mutant [S489A+T251A+K532R] vector by Neutralization antibody assay.

FIG. 14: illustrates In vitro transduction efficiency of T251A-AAV3 mutant vector as compared to WT-AAV3. Equal number of (A) CHO cells, (B) HEK 293 cells are mock-infected or infected with 5×10³ vgs/cell of the AAV3 vectors. Forty eight hours later, the luciferase activity in the cell lysate is measured using a commercial kit (BioVision Inc, Milpitas, Calif., USA) in a GlowMax™ 20/20 luminometer (Promega, WI, USA). Data is mean of 3 wells for each condition. RLU=Relative luciferase unit.

FIG. 15: illustrates increased transduction efficiency of AAV3 T251A mutant vector in vivo. In vivo bio-luminescence imaging of AAV3 vector administered mice. Animals injected with 5×10¹⁰ vgs of AAV3 Luciferase vectors are imaged 1-week after gene transfer in an IVIS Spect-CT small animal imaging system (Perkin Elmer, Caliper Life Sciences).

FIG. 16: illustrates In vitro transduction efficiency of T251A-AAV4 mutant vector as compared to WT-AAV4. Equal number of CHO cells are mock-infected or infected with 2×10³ vgs/cell of the AAV4 vectors. Forty eight hours later, the luciferase activity in the cell lysate is measured using a commercial kit (BioVision Inc, Milpitas, Calif., USA) in a GlowMax™ 20/20 luminometer (Promega, WI, USA). Data is mean of 3 wells for each condition. RLU=Relative luciferase unit. *p<0.05

FIG. 17: illustrates increased transduction efficiency of AAV5 serine/threonine mutant vectors in vitro and in vivo. (A) In vitro transduction efficiency of AAV5 vectors. CHO cells are either mock-infected or infected with 5×10³ vgs/cell of the different AAV5 vectors. Forty-eight hours post-transduction, cell suspensions are analyzed for EGFP expression by flow cytometry. Representative histograms are shown. The data generated is from mean of triplicate analyses from two independent experiments. (B) Visual comparison of AAV5 S/T/K mutants in comparison to WT-AAV5 vectors. EGFP expression is detected by fluorescence microscopy 4-weeks post-administration of 5×10¹⁰ vector particles/animal of WT-AAV5 or mutant vectors. Representative images of hepatic tissues from four different animals in each group are shown. (C) Analysis of EGFP transcript levels by real-time quantitative PCR. Hepatic RNA isolated from animals injected with WT-AAV5 or AAV5 mutant vectors are analyzed for EGFP expression and the data normalized to the GAPDH reference gene. (D) Estimation of vector genome copies in the liver after AAV5 mediated gene transfer. Genomic DNA is isolated from liver tissue of C57BL/6 mice 4-weeks post vector administration and the viral copy numbers estimated by a quantitative PCR.*p<0.05 Vs WT-AAV5 injected animals.

FIG. 18: illustrates the schematic representation of the serine (S), threonine (T) and lysine (K) residues mutated in AAV5 and their conservation status across other AAV serotypes. VP1 protein sequences from AAV serotypes 1 through 10 are aligned by ClustalW and the conservation status of the each of the target site for mutagenesis is shown in red.

FIG. 19: illustrates increased transduction efficiency of AAV6 T251A mutant vector in vivo. In vivo bio-luminescence imaging of AAV6 vector administered mice. Animals injected with 1×10¹⁰ vgs of AAV6-Luciferase vectors are imaged 1 week after gene transfer in an IVIS Spect-CT small animal imaging system (Perkin Elmer, Caliper Life Sciences). *p<0.05

FIG. 20: illustrates increased transduction efficiency of AAV7 T252A mutant vector in vivo. In vivo bio-luminescence imaging of AAV7 vector administered mice. Animals injected with 5×10¹⁰ vgs of AAV7-Luciferase vectors are imaged 1 week after gene transfer in an IVIS Spect-CT small animal imaging system (Perkin Elmer, Caliper Life Sciences).*p<0.05.

FIG. 21: illustrates the schematic representation of the serine (S), threonine (T) and lysine (K) residues mutated in AAV8 and their conservation status across other AAV serotypes. VP1 protein sequences from AAV serotypes 1 through 10 are aligned by ClustalW and the conservation status of the each of the target site for mutagenesis is shown in red.

FIG. 22: illustrates the phosphodegron in AAV8 capsid structure. (A) The figure shows the capsid protein from AAV8 (PDB id: 2qa0) which is coloured in yellow. S671 (Coloured in cyan) lies in phosphodegron region (652-674) which is coloured in red. The phosphodegron is rich in prolines which are coloured green. The predicted phosphosites (serines and threonine) are shown in red while the predicted ubiquitination sites are in blue. (B) The zoomed in view of phosphodegron region (652-674) containing S671. (C) Comparison of phosphodegrons in AAV8 and AAV2. The figure shows structural superimposition of AAV8 (PDB id: 2qa0) and AAV2 (PDB id: 11p3) coloured in yellow and grey respectively. Phosphodegron in AAV8 coloured in red is equivalent to phosphodegron2 in AAV2 (515-528) which is coloured in green. Phosphodegrons in both AAV2 and 8 are rich in proline residues. The residue S525 in AAV2 (coloured in cyan) lies in the phosphodegron and has been shown to increase the transduction efficiency (Gabriel et al., 2013). (D) The zoomed in view of the phosphodegron2 in AAV2 and AAV8. (E) The zoomed in view of phosphodegron3 of AAV2 in comparison with that in AAV8. The presence of another phosphodegron region in AAV2 (Phosphodegron 3: residues 489-507) which is rich in acidic residues is coloured in purple. S489 and S498 in this region of AAV2 have shown to increase the transduction efficiency in vivo. Whereas, the equivalent region in AAV8 coloured in pink lacks the acidic residues. (F) The figure shows the predicted phosphodegron stretch (122-137) in AAV8 which contains K137 highlighted in yellow. The phosphodegron is rich in proline and are coloured in green. The predicted phosphosites (T138, S149, S153 and S156) are coloured in magenta while the predicted ubiquitination sites (K137, K142 and K143) are coloured in blue.

FIG. 23: illustrates enhanced transduction upon hepatic gene transfer of AAV8 serine and lysine mutant vectors in vivo. (A) Representative images from each animal (n=4 for mock, S279A, S501A and S671A, n=8 for WT and K137R) showing the transgene expression as detected by fluorescence microscopy 4-weeks post-injection with 5×10¹⁰ particles/animal of wild-type or S→A and K→R mutant AAV8 via the tail vein. (B) Quantitative analyses of the data from (A). (C) Representative images from each animal (n=4) showing the transgene expression as detected by fluorescence microscopy 2-weeks post-injection with 5×10¹⁰ particles of wild-type or T→A mutant AAV8 vectors. (D) Quantitative analyses of the data from (C). All the images are taken at the same exposure within each group (A or C) tested (207 milli-secs for A and 1.2 sec for C), gain (n=2) and intensity (n=4) in a fluorescence microscope (Leica CTR6000, GmbH, Germany). (E) EGFP transcript levels normalized to GAPDH expression in murine hepatocytes 4 weeks or (F) 2 weeks post vector administration as measured by quantitative PCR. (G) Comparison of the best performing AAV8 K137R mutant with luciferase as the transgene construct 2 weeks post hepatic gene transfer, with other AAV8 mutants and their quantitation data is shown in H. Animals are injected with 5×10¹⁰ vgs of different AAV8-Luciferase vectors are imaged 2-weeks after gene transfer in an IVIS Spect-CT small animal imaging system (Perkin Elmer, Caliper Life Sciences). Analysis of Variance (ANOVA) is used to compare gene expression between WT or mutant AAV8 treated groups.*p<0.05.

FIG. 24: illustrates superior hF.IX expression of K137R-AAV8 vector in comparison to WT-AAV8 vectors in C57BL/6 mice. Increased h.FIX expression from transgene constructs driven by either hAAT (A) or LP1 (B) promoters from animals injected with K137R-AAV8 vector and compared to the WT-AAV8 vector up to 8 weeks after hepatic gene transfer. *p<0.05 Vs. WT-AAV8 injected mice.

FIG. 25: illustrates superior hF.IX expression of K137R-AAV8 vector in comparison to WT-AAV8 vectors in hemophilia B mice. Increased h.FIX expression is seen from animals injected with K137R-AAV8-FIX vector when compared to the WT-AAV8-FIX vector up to 8 weeks after hepatic gene transfer. *p<0.05 Vs. WT-AAV8-FIX injected mice.

FIG. 26: illustrates reduced ubiquitination of K137R-AAV8 lysine mutant vector in comparison to WT-AAV8 vector. (A) Approximately 3×10⁸ viral particles of WT-AAV8 and K137R-AAV8 vectors are denatured at 95° C. for 5 minutes. The denatured viral particles are then used to perform the ubiquitin conjugation assay according to the manufacturer's protocol. The processed samples are electrophoresed on a 4-20% denaturing polyacrylamide gel and the ubiquitination pattern detected by immunoblotting using an anti-ubiquitin antibody. The mono-to-polyubiquitin conjugates are detected as a smear at molecular weight >150 Kda. (B) Approximately 3×10⁸ viral particles of WT-AAV8 and K137R-AAV8 vectors are denatured with RIPA buffer containing protease inhibitor cocktail at 95° C. for 10 minutes. The samples are resolved in a 4-20% denaturing polyacrylamide gel and the VP1 (87 Kda), VP2 (72 KDa) and VP3 (62 Kda) capsid proteins are detected with AAV clone B1 antibody

FIG. 27: illustrates reduced inflammatory cytokine response of K137R-AAV8 vector in comparison to WT-AAV8 vectors in vivo. Quantitative PCR is used to profile the hepatic expression of key pro-inflammatory cytokines and other markers of innate immune response. The relative fold change in the target gene expression from mice injected with WT-AAV8 and K137R-AAV8 vector in comparison to mock-injected animals are shown. *p<0.05 denotes statistical significance as compared to the WT-AAV8 injected mice.

FIG. 28: illustrates the transduction efficiency of AAV9 vectors in vivo. C57BL6 mice are infected with the WT-AAV9 and the mutant vectors at 5×10¹⁰ vgs per animal. Thirty (T251A) or forty-five (K143R) days post administration the luciferase activity is determined in a small animal imaging system.

FIG. 29: illustrates the comparison of AAVrh.10 wild-type and mutant vectors in vitro in HeLa (A) and AAV293 cells (B). Fold changes in relative luminescence units observed in cells 48-hrs post-infection with WT and the nine AAVrh.10 mutants as analyzed by the luciferase detection assay.

FIG. 30: illustrates the improved transduction efficiency of AAVrh.10-S671A vectors in C57BL/6 mice in comparison to Wild type AAVrh.10 (WT) vectors as demonstrated by bio-luminescence imaging in an IVIS Spect-CT imaging system.

FIG. 31: illustrates the wild type vector construct of AAV1

FIG. 32: illustrates the wild type vector construct of AAV2

FIG. 33: illustrates the wild type vector construct of AAV3

FIG. 34: illustrates the wild type vector construct of AAV4

FIG. 35: illustrates the wild type vector construct of AAV5

FIG. 36: illustrates the wild type vector construct of AAV6

FIG. 37: illustrates the wild type vector construct of AAV7

FIG. 38: illustrates the wild type vector construct of AAV8

FIG. 39: illustrates the wild type vector construct of AAV9

FIG. 40: illustrates the wild type vector construct of AAV 10

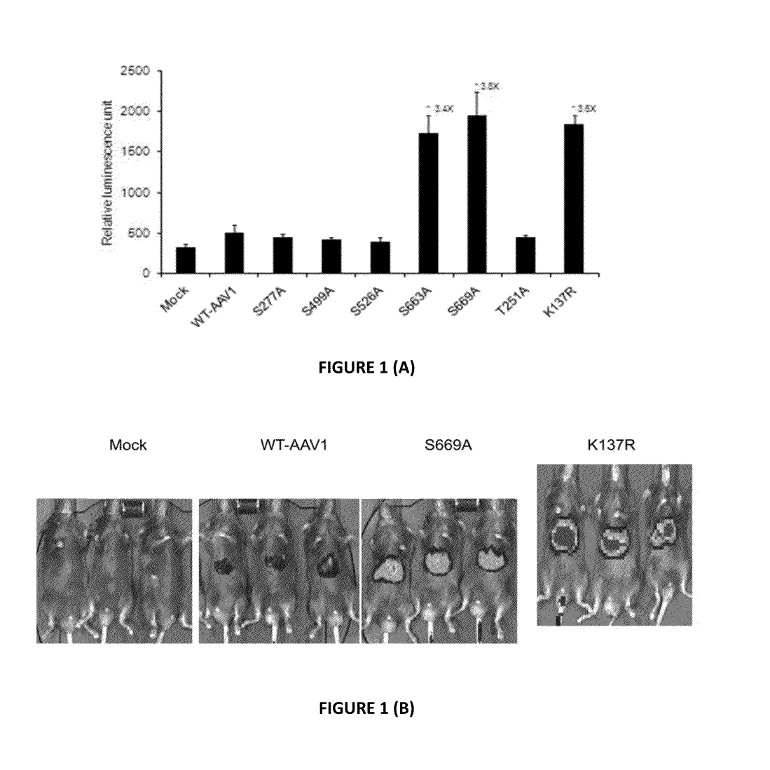

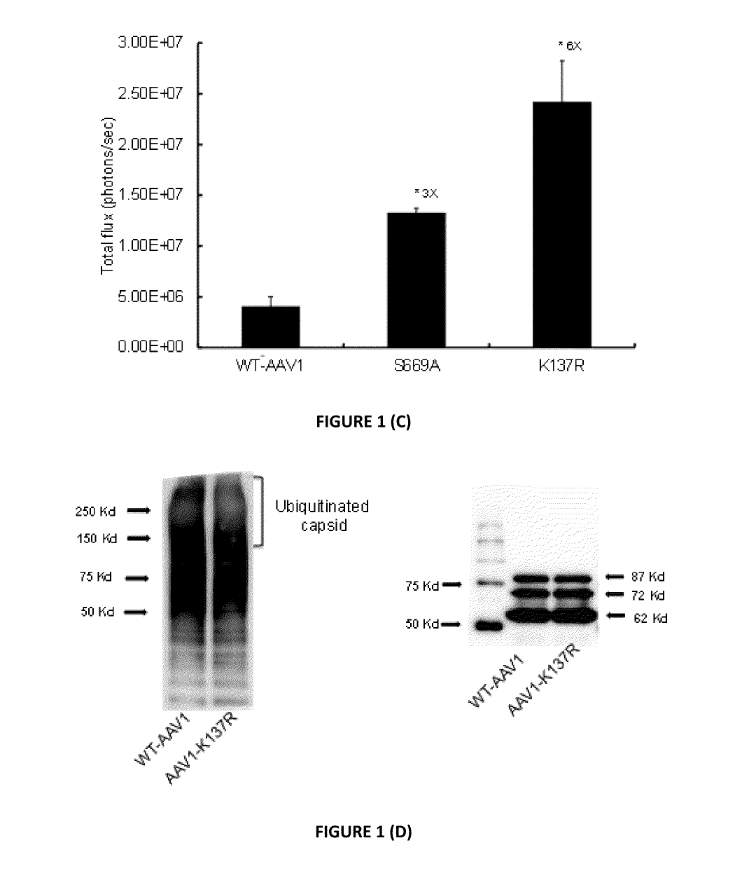

### DETAILED DESCRIPTION OF THE DISCLOSURE

The present disclosure relates to a nucleotide sequence selected from a group comprising SEQ ID Nos. 139 to 148, having mutation at codon selected from a group comprising TCT, TCC, TCA, TCG, AGT, AGC, ACT, ACC, ACA, ACG, AAA and AAG or any combination thereof.

In an embodiment of the present disclosure, the sequence selected from a group comprising SEQ ID Nos. 139 to 148, corresponds to serotypes 1 to 10 respectively, of wild type adeno-associated virus vector.

In another embodiment of the present disclosure, the sequence after mutation is represented by SEQ ID Nos. 149 to 158, respectively with respect to the wild type SEQ ID Nos. 139 to 148.

In yet another embodiment of the present disclosure, the sequence having SEQ ID Nos. 149 to 158, corresponds to mutated serotypes 1 to 10 respectively, of adeno-associated virus vector. In still another embodiment of the present disclosure, the codon TCT, TCC, TCA, TCG, AGT or AGC code for amino acid serine; codon ACT, ACC, ACA or ACG code for amino acid threonine; and the codon AAA or AAG code for amino acid lysine.

In still another embodiment of the present disclosure, the codon TCT, TCC, TCA, TCG, AGT, AGC, ACT, ACC, ACA or ACG is mutated to any codon selected from a group comprising GCT, GCC, GCA or GCG.

In still another embodiment of the present disclosure, the codon AAA or AAG is mutated to any codon selected from a group comprising CGT, CGC, CGA, CGG, AGA or AGG.

In still another embodiment of the present disclosure, the codon GCT, GCC, GCA or GCG code for amino acid alanine; and the codon CGT, CGC, CGA, CGG, AGA or AGG code for amino acid arginine.

In still another embodiment of the present disclosure, mutation in the codon coding for serine or threonine results in replacement of said serine or threonine amino acid with alanine amino acid.

In still another embodiment of the present disclosure, mutation in the codon coding for lysine results in replacement of said lysine amino acid with arginine amino acid.

In still another embodiment of the present disclosure, the mutation in codon TCT, TCC, TCA, TCG, AGT or AGC in any of the SEQ ID Nos. 139 to 148, occurs at position of the corresponding amino acid sequence, said position selected from a group comprising 149, 156, 268, 276, 277, 278, 279, 485, 489, 490, 492, 498, 499, 501, 525, 526, 537, 547, 652, 658, 662, 663, 668, 669 and 671 or any combination thereof.

In still another embodiment of the present disclosure, the mutation in codon ACT, ACC, ACA or ACG in any of the SEQ ID Nos. 139 to 148, occurs at position of the corresponding amino acid sequence, said position selected from a group comprising 107, 108, 138, 245, 251, 252, 328, 454, 503, 654, 671, 674, 701, 713 and 716 or any combination thereof.

In still another embodiment of the present disclosure, the mutation in codon AAA or AAG in any of the SEQ ID Nos. 139 to 148, occurs at position of the corresponding amino acid sequence, said position selected from a group comprising 32, 39, 84, 90, 137, 143, 161, 333, 490, 507, 527, 532, 544 and 652 or any combination thereof.

The present disclosure further relates to a nucleotide sequence selected from a group comprising SEQ ID Nos. 70 to 138, having mutation at codon selected from a group comprising TCT, TCC, TCA, TCG, AGT, AGC, ACT, ACC, ACA, ACG, AAA and AAG or any combination thereof.

In another embodiment of the present disclosure, the sequence selected from a group comprising SEQ ID Nos. 70 to 138, represents wild type primer sequence capable of amplifying nucleotide sequence corresponding to serotypes 1 to 10, of wild type adeno-associated virus vector.

In yet another embodiment of the present disclosure, the codon TCT, TCC, TCA, TCG, AGT, AGC, ACT, ACC, ACA or ACG is mutated to any codon selected from a group comprising GCT, GCC, GCA or GCG.

In still another embodiment of the present disclosure, the codon AAA or AAG is mutated to any codon selected from a group comprising CGT, CGC, CGA, CGG, AGA or AGG.

In still another embodiment of the present disclosure, the sequence having said mutation is selected from a sequence having SEQ ID Nos. 1 to 69.

In still another embodiment of the present disclosure, the mutated sequence act as primer for carrying out site directed mutagenesis for obtaining the mutated nucleotide sequence as mentioned above.

The present disclosure further relates to a method of obtaining a nucleotide sequence selected from a group comprising SEQ ID Nos. 139 to 148, having mutation at codon selected from a group comprising TCT, TCC, TCA, TCG, AGT, AGC, ACT, ACC, ACA, ACG, AAA and AAG or any combination thereof, said method comprising act of introducing mutation in any of nucleotide sequence selected from a group comprising SEQ ID Nos. 139 to 148 through site directed mutagenesis by using the nucleotide sequence as mentioned above.

In another embodiment of the present disclosure, the sequence selected from a group comprising SEQ ID Nos. 139 to 148, corresponds to serotypes 1 to 10 respectively, of wild type adeno-associated virus vector.

The present disclosure also relates to a method of enhancing transduction efficiency, said method comprising act of expressing a target gene in presence of a nucleotide sequence selected from a group comprising SEQ ID Nos. 139 to 148, having mutation at codon selected from a group comprising TCT, TCC, TCA, TCG, AGT, AGC, ACT, ACC, ACA, ACG, AAA and AAG or any combination thereof.

In another embodiment of the present disclosure, the sequence selected from a group comprising SEQ ID Nos. 139 to 148, corresponds to serotypes 1 to 10 respectively, of wild type adeno-associated virus vector.

In still another embodiment of the present disclosure, the transduction efficiency is enhanced with minimizing immunological response when compared with transduction carried out in presence of wild type adeno-associated virus vector having sequence selected from a group comprising SEQ ID Nos. 139 to 148.

The present disclosure also relates to a recombinant cell comprising the nucleotide sequence as mentioned above.

The present disclosure also relates to a method of obtaining the recombinant cell as mentioned above, said method comprising act of introducing the nucleotide sequence as mentioned above to a host cell, to obtain said recombinant cell.

The present disclosure also relates to a kit comprising a nucleotide sequence selected from a group comprising SEQ ID Nos. 70 to 138, having mutation at codon selected from a group comprising TCT, TCC, TCA, TCG, AGT, AGC, ACT, ACC, ACA, ACG, AAA and AAG or any combination thereof.

In another embodiment of the present disclosure, the sequence selected from a group comprising SEQ ID Nos. 70 to 138, represents wild type primer sequence capable of amplifying nucleotide sequence corresponding to serotypes 1 to 10, of wild type adeno-associated virus vector.

The instant disclosure selects specific serine, threonine, lysine residues for modification since a majority these residues are conserved among various known AAV serotypes [AAV1-10]. Further, based on in silico structural prediction, it is identified that certain Ser/Thr/Lys residues are close to or within the “phosphodegrons” which are sites predicted to be targets for host cellular kinase/ubiquitination machinery. Thus, on studying the compatibility of these modifications on AAV2 capsid the residues are specifically mutated as: Serine/Threonine to Alanine and Lysine to Arginine. Further, additional lysine residues are mutated to Arginine on the AAV2 capsid as predicted by UBPred software that calculates the likelihood of an amino acid sequence likely to be ubiquitinated. Upon experimental analysis, it is found that various serine, threonine and lysine mutants of AAV serotypes show an increase in transgene expression in vitro as well as higher transduction efficiency when administered to murine models in vivo. This demonstrates a superior AAV vector system for efficacious gene transfer in vivo. It is found that combining the various serine/threonine and lysine as multiple mutants further augments the efficiency of AAV mediated gene therapy.

In an embodiment, the instant disclosure presents that AAV serotypes which are generally targeted for destruction in the cytoplasm by the host-cellular kinase/ubiquitination/proteasomal degradation machinery, are modified at the serine/threonine kinase targets or ubiquitination targets (lysine) on AAV capsid, which improves its transduction efficiency.

In an embodiment of the present disclosure, in-silico structural analysis of the AAV2 capsid enables identification of three protein motifs (phosphodegrons) which are the phosphorylation sites recognized as degradation signals by ubiquitin ligases (Table 1).

Table 1 describes the location and amino acid sequence of the three phosphodegrons in the AAV2 capsid. The predicted phosphorylation and ubiquitination sites (shown in bold font) that are highly conserved among all the serotypes of AAV within the phosphodegron region (shown enlarged) are listed. All the three phosphodegrons are solvent accessible as shown by its high average solvent accessibility.

| Phospho- | Amino acid position | Amino acid sequence | Average solvent |
|---|---|---|---|
| degron | (NCBI numbering) | (N-C terminus) | accessibility (%) |
| 1 | 525-564 | SHKDDEEKFFPQSGVLIFGKQGSE | 23.6 |
| | | KTNVDIEKVMITDEEE | |
| 2 | 652-665 | PVPANPSTTFSAAK | 35.0 |
| 3 | 489-507 | SKTSADNNNSEYSWTGATK | 24.5 |

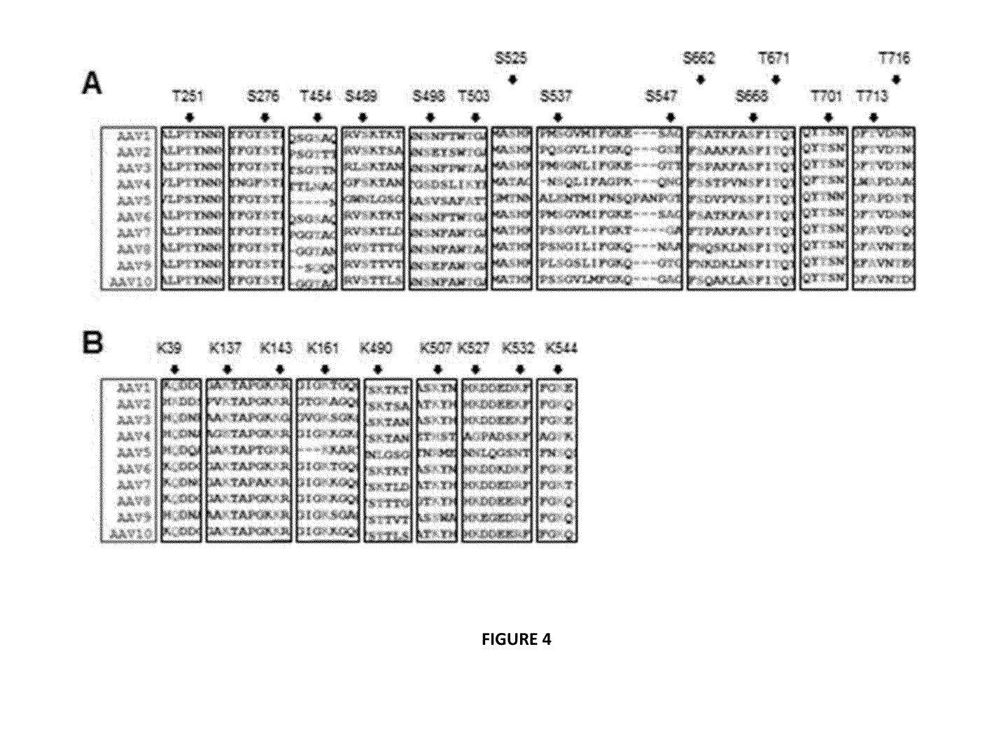

Point-mutations are generated in each of the serine/threonine residues to Alanine residues within or around the three phosphodegrons' residues on AAV2 capsid protein viz. S276A, S489A, S498A, S525A, S537A, S547A, S662A, S668A, T251A, T454A, T503A, T671A, T701A, T713A, and T716A (illustrated in FIG. 4).

In an embodiment of the present disclosure, the lysine residues are modified to arginine residues viz. K39R, K137R, K143R, K161R, K490R, K507R, K527R, K532R, and K544R.

In an embodiment of the present disclosure, the mutant AAV2 vectors have multiple combinations of AAV2 serine/threonine/arginine mutants as illustrated in Table 2 below.

TABLE 2
Mutant AAV2 vectors having multiple combinations of
AAV2 serine/threonine/arginine mutations.
DOUBLE MUTANTS

| | |
|---|---|
| 1 | AAV2 K490R + K532R |
| 2 | AAV2 K527R + K532R |
| 3 | AAV2 K532R + S489A |
| 4 | AAV2 T251A + K532R |

TRIPLE MUTANT

| | |
|---|---|
| 1 | AAV2 T251A + K532R + S489A |
| 2 | AAV2 S489A + S498A + K532R |

In an embodiment of the present disclosure, increased transduction efficiency of the AAV2 mutant vectors translates into enhanced therapeutic benefit in patients undergoing AAV-mediated gene-therapy. As a result, this dramatically reduces the requirement of multiple-vector administration or attenuates the host immune response due to lower mutant AAV vector doses administered, thus potentially improving the safety and efficacy of gene-therapy in humans.

Wide-Applicability:

A majority of serine/threonine/lysine residues targeted for mutation in phosphodegrons (1-3) of AAV serotype-2 are conserved in other AAV serotypes (AAV1-10). A similar serine/threonine→alanine or lysine→arginine mutations in AAV-1, 3, 4, 5, 6, 7, 8, 9, 10 serotypes improves the transduction efficiency from these serotypes as well, translating into broad applicability in the gene therapy field for various diseases.

In an embodiment of the present disclosure, the mutant AAV vectors thus offer the following competitive advantages:
(i) improve efficiency of gene delivery;
(ii) lower the costs of gene therapy as low doses of vectors will be administered; and
(iii) promote safety of the AAV vectors by limiting dose-dependent immune-toxicity seen with conventional WT-AAV vectors.

In another embodiment of the present disclosure, each of the S/T/K residues identified in the vicinity of phosphodegron is mutated either as a single mutant, double mutant or multiple mutants.

In another embodiment of the present disclosure, vast majority of S/T/K mutant capsids will not affect the vector packaging efficiency.

In another embodiment of the present disclosure, about 15 S/T→A mutant AAV vectors tested for their transduction efficiency at a multiplicity of infection (MOI) in HeLa cells, out of which, 11 had a significantly higher increase in EGFP-positive cells (63-97%) compared with AAV-WT vector-infected cells (41%) by FACS analysis.

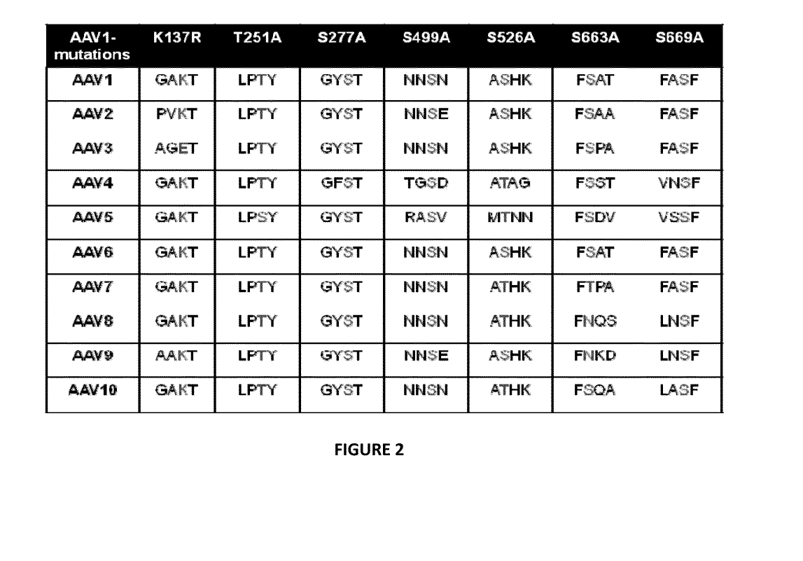

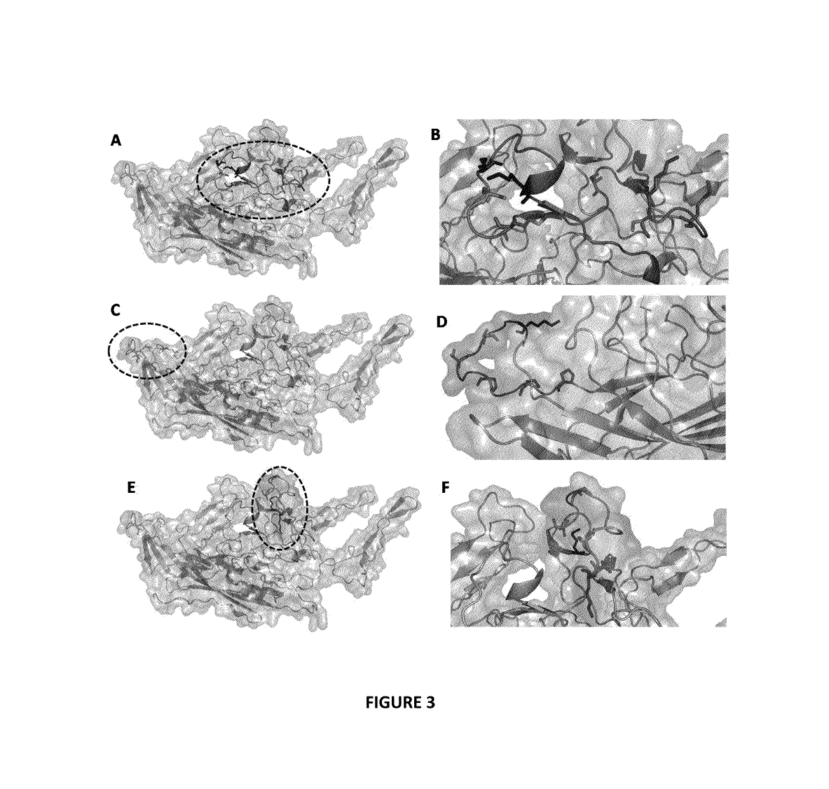

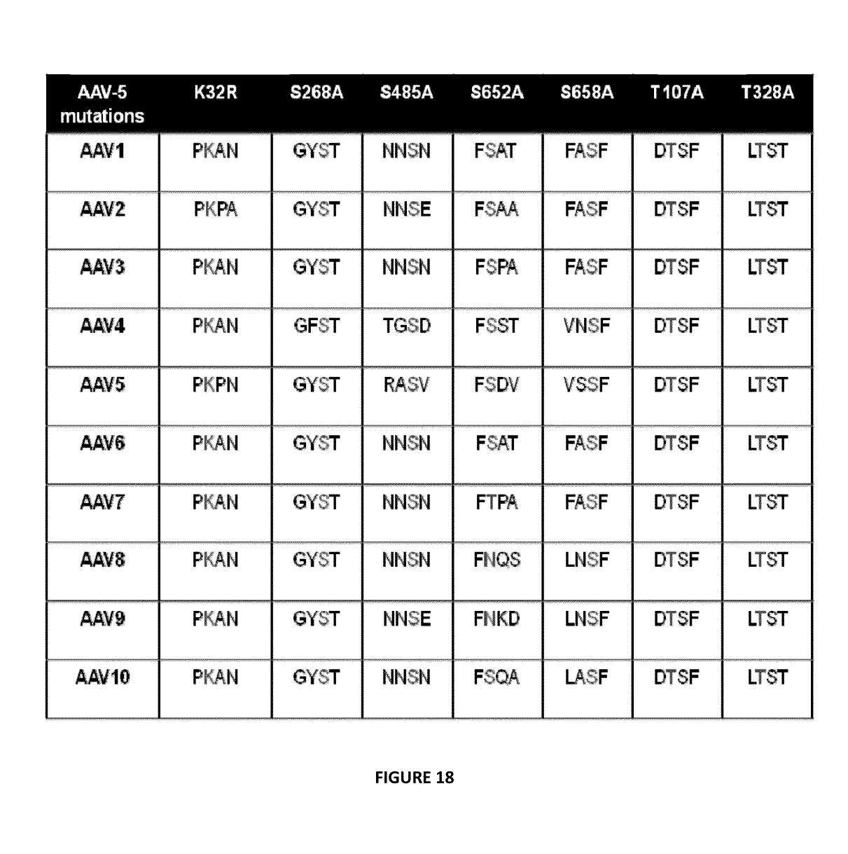

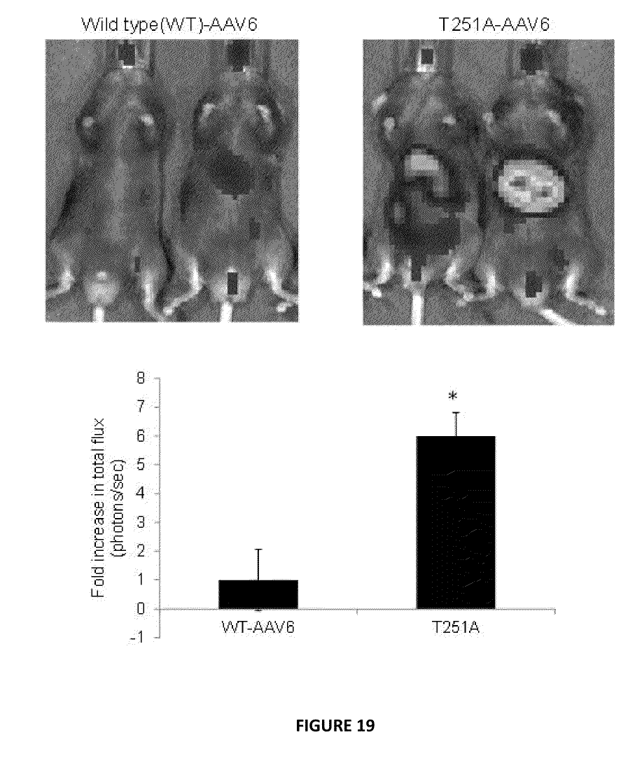

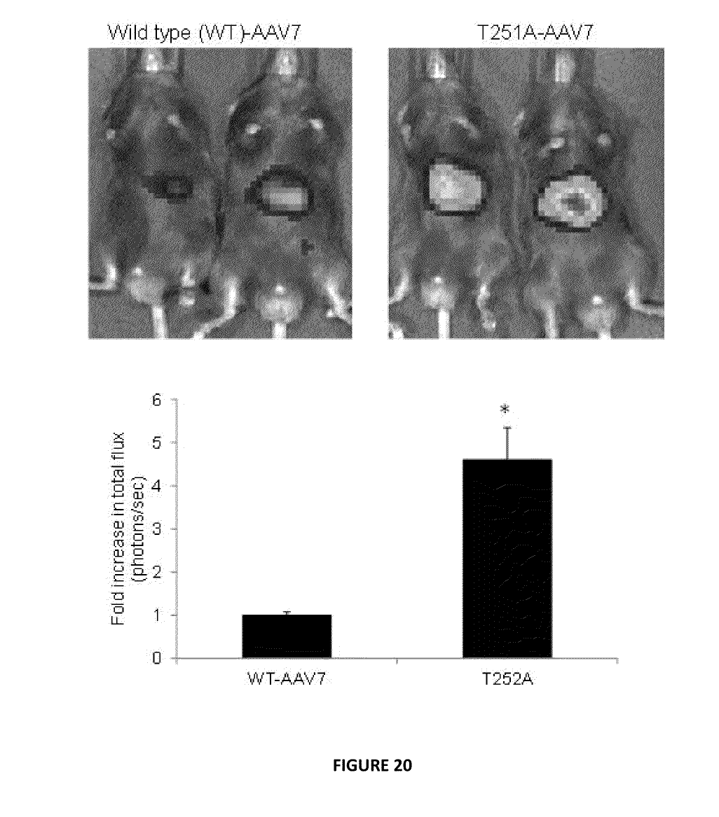

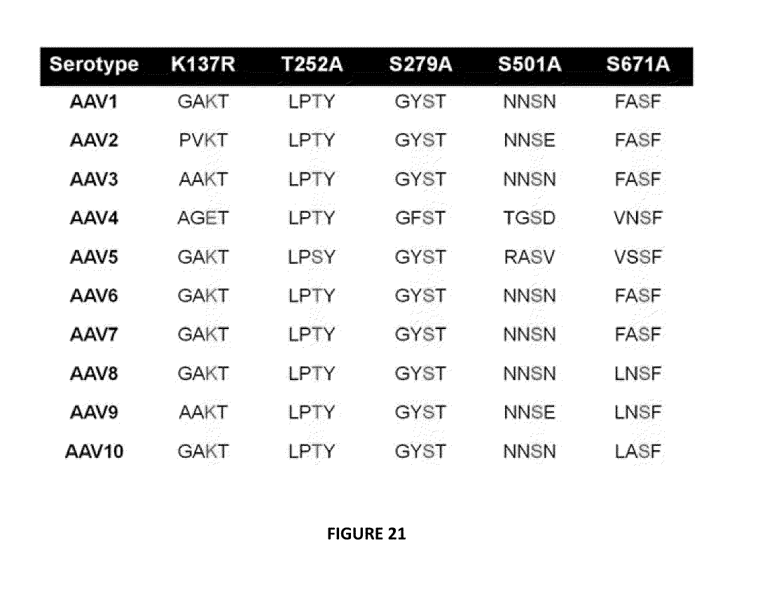

In another embodiment of the present disclosure, conservation of a residue across AAV serotypes is considered an added advantage in selection for mutation of the capsid protein, which is illustrated in FIGS. 2, 4, 18 and 21.

In another embodiment of the present disclosure, the S/T/K mutant AAV vectors have an ability to bypass natural neutralizing antibodies to WT-AAV2, and combined with its low seroprevalence in humans.

In another embodiment of the present disclosure, S/T/K residues are about 19.2% on the capsid protein of AAV2 capsid and most S/T/K mutations within AAV2 or other AAV1-10 serotypes are likely to increase transduction efficiency.

In another embodiment of the present disclosure, mutation of S/K residues enhanced the liver-directed transgene expression across all AAV serotypes.

In an embodiment, the methodology employed to arrive at Ser/Thr/Lys mutants across all serotypes is Site-directed mutagenesis as provided by Example 1 herein. Details of primers used for site-directed mutagenesis of specific Serine/threonine to Alanine and Lysine to Arginine residues in AAV serotypes is provided in Tables 3 to 12 below.

TABLE 3
Details of primers used for site-directed mutagenesis of specific Serine/
threonine to Alanine and Lysine to Arginine residues in AAV1 capsid

| | | | CHANGE IN |
|---|---|---|---|
| | | NUCLEOTIDE | RESTRICTION |
| RESIDUE | SEQUENCE (5′ to 3′) | CHANGE | ENZYME |
| AAV1- | Wild Type Primer Sequence: | AGC→GCC | C→A NcoI |
| S277A | CAACCACTACTTCGGCTACAGCACCCCCTGGGGG | | appears |
| | TATTTTG (SEQ ID 70) | | |
| | Mutant Primer Sequence:- | | |
| | CAACCACTACTTCGGCTACGCCACCCCATGGGGG | | |
| | TATTTTG (SEQ ID 1) | | |
| AAV1- | Wild Type Primer Sequence:- | AGC→GCC | No silent |
| S499A | CAAAAACAGACAACAACAACAGCAATTTTACCTG | | mutation |
| | GACTGGTG (SEQ ID 71) | | |
| | Mutant Primer Sequence:- | | |
| | CAAAAACAGACAACAACAACGCCAATTTTACCTG | | |
| | GACTGGTG (SEQ ID 2) | | |
| AAV1- | Wild Type Primer Sequence:- | TCA→GCA | T→C NcoI |
| S526A | GCACTGCTATGGCCTCACACAAAGACGAC (SEQ ID | | appears |
| | 72) | | |
| | Mutant Primer Sequence:- | | |
| | GCACTGCCATGGCCCGCACACAAAGACGAC(SEQ ID | | |
| | 3) | | |
| AAV1- | Wild Type Primer Sequence:- | TCA→GCA | G→C SacII |
| S663A | CGAATCCTCCGGCGGAGTTTTCAGCTACAAAGTTT | | appears |
| | GCTTC (SEQ ID 73) | | |
| | Mutant Primer Sequence:- | | |
| | CGAATCCTCCCGCGGAGTTTGCAGCTACAAAGTTT | | |
| | GCTTC (SEQ ID 4) | | |
| AAV1- | Wild Type Primer Sequence:- | TCA→GCA | BsmI appears |
| S669A | TTTCAGCTACAAAGTTTGCTTCATTCATCACCCAA | | |
| | TACTCC (SEQ ID 74) | | |
| | Mutant Primer Sequence:- | | |
| | TTTCAGCTACAAAGTTTGCTGCATTCATCACCCAA | | |
| | TACTCC (SEQ ID 5) | | |
| AAV1- | Wild Type Primer Sequence:- | ACC→GCC | C→G NaeI |
| T251A | CCTGGGCCTTGCCCACCTACAATAACCACC (SEQ | | appears |
| | ID 75) | | |
| | Mutant Primer Sequence:- | | |
| | CCTGGGCCTTGCCGCACCTACAATAACCACC (SEQ | | |
| | ID 6) | | |
| AAV1- | Wild Type Primer Sequence:- | AAG→AGA | T→G, BplI |
| K137R | CTGGTTGAGGAAGGCGCTAAGACGGCTCCTGGAA | | appears |
| | AGAAAC (SEQ ID 76) | | |
| | Mutant Primer Sequence:- | | |
| | CTGGTTGAGGAAGGCGCGAGAACGGCTCCTGGAA | | |
| | AGAAAC (SEQ ID 7) | | |

TABLE 4
describes the details of primers used for site directed mutagenesis in AAV2 and the
change in nucleotide and restriction pattern of silent mutations incorporated within
the primers.

| | | | CHANGE IN |
|---|---|---|---|
| | | Nucleotide | RESTRICTION |
| Residue | Sequence (5′ to 3′) | change | ENZYME |
| S276A | Wild type Primer sequence: | AGC→GCC | PflM1 appears |
| | CACTACTTTGGCTACAGCACCCCTTGGGGGTATTTTG | | |
| | ACTTC (SEQ ID 77) | | |
| | Mutant Primer sequence: | | |
| | CACTACTTTGGCTACGCCACCCCTTGGGGGTATTTTG | | |
| | ACTTC (SEQ ID 8) | | |
| S489A | Wild type Primer sequence: | TCA→GCT | Alu1 appears |
| | TACCGCCAGCAGCGAGTATCAAAGACATCTGCGG | | |
| | (SEQ ID 78) | | |
| | Mutant Primer sequence | | |
| | TACCGCCAGCAGCGAGTAGCTAAGACATCTGCGG | | |
| | (SEQ ID 9) | | |
| S498A | Wild type Primer sequence: | AGT→GCT | G→A Pst1 appears |
| | AAGACATCTGCGGATAACAACAACAGTGAATACTCG | | |
| | TGGACT (SEQ ID 79) | | |
| | Mutant Primer sequence | | |
| | AAGACATCTGCAGATAACAACAACGCTGAATACTCG | | |
| | TGGACT (SEQ ID 10) | | |
| S525A | Wild type primer sequence: | AGC→GCC | C→G NcoI |
| | CCGGGCCCGGCCATGGCAAGCCACAAGGACG (SEQ | | disappears |
| | ID 80) | | |
| | Mutant primer sequence: | | |
| | CCGGGCCCGGCGATGGCAGCCCACAAGGACG (SEQ | | |
| | ID 11) | | |
| S537A | Wild type primer sequence: | AGC→GCC | BsrBI disappears |
| | AGAAAAGTTTTTTCCTCAGAGCGGGGTTCTCATCTTT | | |
| | GGG (SEQ ID 81) | | |
| | Mutant primer sequence: | | |
| | AGAAAAGTTTTTTCCTCAGGCCGGGGTTCTCATCTTT | | |
| | GGG (SEQ ID 12) | | |
| S547A | Wild type primer sequence: | TCA→GCC | Sfo1 appears |
| | CATCTTTGGGAAGCAAGGCTCAGAGAAAACAAATGT | | |
| | GGACA (SEQ ID 82) | | |
| | Mutant primer sequence: | | |
| | CATCTTTGGGAAGCAAGGCGCCGAGAAAACAAATGT | | |
| | GGACA (SEQ ID 13) | | |
| S662A | Wild type primer sequence: | AGT→GCT | AcuI disappears |
| | AATCCTTCGACCACCTTCAGTGCGGCAAAGTTTGCTT | | |
| | C (SEQ ID 83) | | |
| | Mutant primer sequence: | | |
| | AATCCTTCGACCACCTTCGCTGCGGCAAAGTTTGCTT | | |
| | C (SEQ ID 14) | | |
| S668A | Wild type primer sequence: | TCC→GCC | T→G AccI appears |
| | GCGAATCCTTCGACCACCTTCAGTGCGGCAAAGTTT | | |
| | GCTTCCTTCATCACA (SEQ ID 84) | | |
| | Mutant primer sequence: | | |
| | GCGAATCCGTCGACCACCTTCAGTGCGGCAAAGTTT | | |
| | GCTGCCTTCATCACA (SEQ ID 15) | | |
| T251A | Wild type primer sequence: | ACC→GCC | C→G NaeI appears |
| | ACCCGAACCTGGGCCCTGCCCACCTACAACAACCAC | | |
| | CTCTAC (SEQ ID 85) | | |
| | Mutant primer sequence: | | |
| | ACCCGAACCTGGGCCCTGCCGGCCTACAACAACCAC | | |
| | CTCTAC (SEQ ID 16) | | |
| T454A | Wild type primer sequence: | ACC→GCC | G→A Afl II appears |
| | TATTACTTGAGCAGAACAAACACTCCAAGTGGAACC | | |
| | ACCACGCAGTCAAGG (SEQ ID 86) | | |
| | Mutant primer sequence: | | |
| | TATTACTTAAGCAGAACAAACACTCCAAGTGGAGCC | | |
| | ACCACGCAGTCAAGG (SEQ ID 17) | | |
| T503A | Wild type primer sequence: | ACT→GCT | G→A PstI appears |
| | ACATCTGCGGATAACAACAACAGTGAATACTCGTGG | | |
| | ACTGGAGCTACCAAG (SEQ ID 87) | | |
| | Mutant primer sequence: | | |
| | ACATCTGCAGATAACAACAACAGTGAATACTCGTGG | | |
| | GCTGGAGCTACCAAG (SEQ ID 18) | | |
| T671A | Wild type primer sequence: | ACA→GCA | A→C ApaBI |
| | TTCAGTGCGGCAAAGTTTGCTTCCTTCATCACACAGT | | disappears |
| | ACTCCACGGGA (SEQ ID 88) | | |
| | Mutant primer sequence: | | |
| | TTCAGTGCGGCCAAGTTTGCTTCCTTCATCGCACAGT | | |
| | ACTCCACGGGA (SEQ ID 19) | | |
| T701A | Wild type primer sequence: | ACT→GCT | TatI disappears |
| | CGCTGGAATCCCGAAATTCAGTACACTTCCAACTAC | | |
| | AACAAGTCTGTT (SEQ ID 89) | | |
| | Mutant primer sequence: | | |
| | CGCTGGAATCCCGAAATTCAGTACGCTTCCAACTAC | | |
| | AACAAGTCTGTT (SEQ ID 20) | | |
| T713A | Wild type primer sequence: | ACT→GCT | G→C SalI appears |
| | GTTAATGTGGACTTTACTGTGGACACTAATGGCGTG | | |
| | TATTCAGAG (SEQ ID 90) | | |
| | Mutant primer sequence: | | |
| | GTTAATGTGGACTTTGCTGTCGACACTAATGGCGTG | | |
| | TATTCAGAG (SEQ ID 21) | | |
| T716A | Wild type primer sequence: | ACT→GCT | G→A AccI appears |
| | AAGTCTGTTAATGTGGACTTTACTGTGGACACTAAT | | |
| | GGCGTGTATTCA (SEQ ID 91) | | |
| | Mutant primer sequence: | | |
| | AAGTCTGTTAATGTAGACTTTACTGTGGACGCTAAT | | |
| | GGCGTGTATTCA (SEQ ID 22) | | |
| K39R | Wild type primer sequence: | AAG→AGG | G→A BsrBI appears |
| | CCAAAGCCCGCAGAGCGGCATAAGGACGACAGCAG | | |
| | GGGTCTTGTG (SEQ ID 92) | | |
| | Mutant primer sequence: | | |
| | CCAAAGCCCGCAGAACGGCATAGGGACGACAGCAG | | |
| | GGGTCTTGTG (SEQ ID 23) | | |
| K137R | Wild type primer sequence: | AAG→AGG | T→C SmaI appears |
| | GGCCTGGTTGAGGAACCTGTTAAGACGGCTCCGGGA | | |
| | AAAAAGAGG (SEQ ID 93) | | |
| | Mutant primer sequence: | | |
| | GGCCTGGTTGAGGAACCTGTTAGGACGGCCCCGGGA | | |
| | AAAAAGAGG (SEQ ID 24) | | |
| K143R | Wild type primer sequence: | AAG→AGG | T→C SmaI appears |
| | CCTGTTAAGACGGCTCCGGGAAAAAAGAGGCCGGT | | |
| | AGAGCAC (SEQ ID 94) | | |
| | Mutant primer sequence: | | |
| | CCTGTTAAGACGGCCCCGGGAAAAAGGAGGCCGGT | | |
| | AGAGCAC (SEQ ID 25) | | |
| K161R | Wild type primer sequence: | AAG→AGG | G→C NaeI appears |
| | GACTCCTCCTCGGGAACCGGAAAGGCGGGCCAGCA | | |
| | GCCTGCAAGAAAAAGATTG (SEQ ID 95) | | |
| | Mutant primer sequence: | | |
| | GACTCCTCCTCGGGAACCGGAAGGGCCGGCCAGCAG | | |
| | CCTGCAAGAAAAAGATTG (SEQ ID 25) | | |
| K490R | Wild type primer sequence: | AAG→AGG | G→A PstI disappears |
| | CAGCAGCGAGTATCAAAGACATCTGCGGATAACAAC | | |
| | AAC (SEQ ID 96) | | |
| | Mutant primer sequence: | | |
| | CAGCAGCGAGTATCAAGGACATCTGCAGATAACAAC | | |
| | AAC (SEQ ID 27) | | |
| K507R | Wild type primer sequence: | AAG→AGA | Acc65I disappears |
| | TGGACTGGAGCTACCAAGTACCACCTCAATGGCAGA | | |
| | GAC (SEQ ID 97) | | |
| | Mutant primer sequence: | | |
| | TGGACTGGAGCTACCAGATACCACCTCAATGGCAGA | | |
| | GAC (SEQ ID 28) | | |
| K527R | Wild type primer sequence: | AAG→AGG | C→A NcoI |
| | CCGGGCCCGGCCATGGCAAGCCACAAGGACGATGA | | disappears |
| | AGAAAAG (SEQ ID 98) | | |
| | Mutant primer sequence: | | |
| | CCGGGCCCGGCAATGGCAAGCCACAGGGACGATGA | | |
| | AGAAAAG (SEQ ID 29) | | |
| K532R | Wild type primer sequence: | AAG→AGG | G→A BsrBI |
| | CACAAGGACGATGAAGAAAAGTTTTTTCCTCAGAGC | | disappears |
| | GGGGTTCTC (SEQ ID 99) | | |
| | Mutant primer sequence: | | |
| | CACAAGGACGATGAAGAAAGGTTTTTTCCTCAAAGC | | |
| | GGGGTTCTC (SEQ ID 30) | | |
| K544R | Wild type primer sequence: | AAG→AGA | G→T AccI appears |
| | GTTCTCATCTTTGGGAAGCAAGGCTCAGAGAAAACA | | |
| | AATGTG (SEQ ID 100) | | |
| | Mutant primer sequence: | | |
| | GTTCTCATCTTTGGTAGACAAGGCTCAGAGAAAACA | | |
| | AATGTG (SEQ ID 31) | | |

TABLE 5
describes the details of primers used for site-directed mutagenesis
of specific threonine to alanine residue in AAV3 capsid

| | | | Change in |
|---|---|---|---|
| | | Nucleotide | Restriction |
| Residue | Sequence (5′→3′) | Change | Enzyme site |
| T251A | Wild type primer Sequence | ACT→GCT | No change in |
| | CCTGGGCCCTGCCCACTTACAACAACCATC | | Restriction |
| | Mutated primer Sequnce (SEQ ID 101) | | Enzyme site |
| | CCTGGGCCCTGCCCGCTTACAACAACCATC | | |
| | (SEQ ID 32) | | |

TABLE 6
Details of primers used for site-directed mutagenesis of specific
threonine to alanine residue in AAV4 capsid

| | | | Change in |
|---|---|---|---|
| | | Nucleotide | Restriction |
| Residue | Sequence (5′→3′) | Change | Enzyme Site |
| T245A | Wild type primer Sequence | ACC→GCC | No Change in |
| | CCTGGGTCTTGCCCACCTACAACAACCAC | | Restriction |
| | (SEQ ID 102) | | Enzyme site |
| | Mutated primer Sequnce | | |
| | CCTGGGTCTTGCCCGCCTACAACAACCAC | | |
| | (SEQ ID 33) | | |

TABLE 7
describes the details of primers used for site-directed mutagenesis of specific
Serine/threonine to Alanine and Lysine to Arginine residues in AAV5

| | | | CHANGE IN |
|---|---|---|---|
| | | NUCLEOTIDE | RESTRICTION |
| RESIDUE | SEQUENCE (5′ to 3) | CHANGE | ENZYME |
| AAV5- | Wild Type Primer Sequence:- | AGC→GCC | No silent |
| S268A | AACGCCTACTTTGGATACAGCACCCCCTGGGGGT | | mutations |
| | AC (SEQ ID 103) | | |
| | Mutant Primer Sequence:- | | |
| | GCCTACTTTGGATACGCCACCCCCTGGGGG (SEQ | | |
| | ID 34) | | |
| AAV5- | Wild Type Primer Sequence:- | AGT→GCT | TSPR I |
| S485A | CGGGGTCAACCGCGCCAGTGTCAGCGCCTTCGCC | | disappears |
| | (SEQ ID 104) | | |
| | Mutant Primer Sequence:- | | |
| | GGTCAACCGCGCCCiCTGTCAGCGCCTTC (SEQ ID | | |
| | 35) | | |
| AAV5- | Wild Type Primer Sequence:- | TCG→GCG | HPYI 88I |
| S652A | GAAATATCACCAGCTTCTCGGACGTGCCCGTCAGC | | disappears |
| | (SEQ ID 105) | | |
| | Mutant Primer Sequence:- | | |
| | ATATCACCAGCTTCGCGGACGTGCCCGTC (SEQ ID | | |
| | 36) | | |
| AAV5- | Wild Type Primer Sequence:- | AGC→GCC | ALU I |
| S658A | TCGGACGTGCCCGTCAGCAGCTTCATCACCCAGTA | | disappears |
| | CAG (SEQ ID 106) | | |
| | Mutant Primer Sequence:- | | |
| | GACGTGCCCGTCAGCGCCTTCATCACCCAGTA | | |
| | (SEQ ID 37) | | |
| AAV5- | Wild Type Primer Sequence:- | AAA→AGA | No silent |
| K32R | AAGCGGGCCCACCGAAACCAAAACCCAATCAGCA | | mutation |
| | G (SEQ ID 107) | | |
| | Mutant Primer Sequence:- | | |
| | CGGGCCCACCGAAACCAAGACCCAATCAG (SEQ | | |
| | ID 38) | | |
| AAV5- | Wild Type Primer Sequence:- | ACA→GCA | MWO I appears |
| T107A | AAGCTCGCCGACGACACATCCTTCGGGGAA (SEQ | | |
| | ID 108) | | |
| | Mutant Primer Sequence:- | | |
| | CTCGCCGACGACGCATCCTTCGGGG (SEQ ID 39) | | |
| AAV5- | Wild Type Primer Sequence:- | ACC→GCC | HPH I |
| T328A | CGCCAACAACCTCACCTCCACCGTCCAAGT (SEQ | | disappears |
| | ID 109) | | |
| | Mutant Primer Sequence:- | | |
| | CGCCAACAACCTCGCCTCCACCGTCCA (SEQ ID 40) | | |

TABLE 8
describes the details of primers used for site-directed mutagenesis
of specific threonine to alanine residue in AAV6 capsid

| | | | Change in |
|---|---|---|---|
| | | Nucleotide | Restriction |
| Residue | Sequence (5′→3′) | Change | Enzyme site |
| T251A | Wild type primer Sequence | ACC→GCC | C→G, Nae I |
| | CATGGGCCTTGCCCACCTATAACAACCAC | | appears |
| | (SEQ ID 110) | | |
| | Mutated primer Sequence | | |
| | CATGGGCCTTGCCGGCCTATAACAACCA | | |
| | (SEQ ID 41) | | |

TABLE 9
describes the details of primers used for site-directed mutagenesis
of specific serine to alanine residue in AAV7 capsid

| | | Nucleotide | Change in Restriction |
|---|---|---|---|
| Residue | Sequence (5′→3′) | Change | Enzyme site |
| T252A | Wild type primer Sequence | ACC→GCC | No Change in |
| | CTGGGCCCTGCCCACCTACAACAACCA | | Restriction Enzyme site |
| | (SEQ ID 111) | | |
| | Mutated primer Sequence | | |
| | CTGGGCCCTGCCCGCCTACAACAACCA | | |
| | (SEQ ID 42) | | |

TABLE 10
describes the details primers used for site directed mutagenesis in AAV8
capsid and the change in nucleotide and restriction pattern of silent
mutation obtained with the primers.

| | | | CHANGE IN |
|---|---|---|---|
| | | | RESTRICTION |
| | | NUCLEOTIDE | ENZYME DUE TO |
| RESIDUE | SEQUENCE (5′ to 3′) | CHANGE | SILENT MUTATION |
| S149A | Wild Type Primer Sequence:- | TCA→GCA | No silent mutation |
| | GAGACCGGTAGAGCCATCACCCCAGCGT | | |
| | TCTCC (SEQ ID 112) | | |
| | Mutant Primer Sequence:- | | |
| | GAGACCGGTAGAGCCAGCACCCCAGCG | | |
| | TTCTCC (SEQ ID 43) | | |
| S156A | Wild Type Primer Sequence:- | TCC→GCC | No silent mutation |
| | CCCAGCGTTCTCCAGACTCCTCTACGGG | | |
| | CATCGGC (SEQ ID 113) | | |
| | Mutant Primer Sequence:- | | |
| | CCCAGCGTTCTCCAGACGCCTCTACGGG | | |
| | CATCGGC (SEQ ID 44) | | |
| S279A | Wild Type Primer Sequence: | AGC→GCC | SfcI disappears |
| | CTACTTCGGCTACAGCACCCCCTGGGGG | | |
| | (SEQ ID 114) | | |
| | Mutant Primer Sequence:- | | |
| | CTACTTCGGCTACGCCACCCCCTGGGGG | | |
| | (SEQ ID 45) | | |
| S501A | Wild Type Primer Sequence: | AGC→GCC | G-T AgeI appears |
| | GACAACCGGGCAAAACAACAATAGCAA | | |
| | CTTTGCCTGGACT (SEQ ID 115) | | |
| | Mutant Primer Sequence:- | | |
| | GACAACCGGTCAAAACAACAATGCCAA | | |
| | CTTTGCCTGGACT (SEQ ID 46) | | |
| S671A | Wild Type Primer Sequence:- | TCT→GCT | T→A AluI disappears |
| | CCTTCAACCAGTCAAAGCTGAACTCTTT | | |
| | CATCACGCAATACA (SEQ ID 116) | | |
| | Mutant Primer Sequence:- | | |
| | CCTTCAACCAGTCAAAGCAGAACGCTTT | | |
| | CATCACGCAATACA (SEQ ID 47) | | |
| T138A | Wild Type Primer Sequence:- | ACG→GCG | No silent mutation |
| | TTGAGGAAGGCGCTAAGACGGCTCCTGG | | |
| | AAAGAAG (SEQ ID 117) | | |
| | Mutant Primer Sequence:- | | |
| | TTGAGGAAGGCGCTAAGGCGCTCCTG | | |
| | GAAAGAAG (SEQ ID 48) | | |
| T252A | Wild type primer sequence: | ACC→GCC | C→G NaeI appears |
| | ACCCGAACCTGGGCCCTGCCCACCTACA | | |
| | ACAACCACCTCTAC (SEQ ID 118) | | |
| | Mutant primer sequence: | | |
| | ACCCGAACCTGGGCCCTGCCGGCCTACA | | |
| | ACAACCACCTCTAC (SEQ ID 49) | | |
| T654A | Wild Type Primer Sequence:- | ACG→GCG | T→A appearance of |
| | AGATCCTGATCAAGAACACGCCTGTACC | | BstUI, Hinp1I, |
| | TGCGGATCCTCC (SEQ ID 119) | | HhaI, BsrI |
| | Mutant Primer Sequence:- | | |
| | AGATCCTGATCAAGAACGCGCCAGTACC | | |
| | TGCGGATCCTCC (SEQ ID 50) | | |
| T674A | Wild Type Primer Sequence:- | ACG→GCG | No silent mutation |
| | AAAGCTGAACTCTTTCATCACGCAATAC | | |
| | AGCACCGGACA (SEQ ID 120) | | |
| | Mutant Primer Sequence:- | | |
| | AAAGCTGAACTCTTTCATCGCGCAATAC | | |
| | AGCACCGGACA (SEQ ID 51) | | |
| K137R | Wild Type Primer Sequence:- | AAG→AGG | C→G HhaI disappears |
| | GGTTGAGGAAGGCGCTAAGACGGCTCCT | | |
| | GG (SEQ ID 121) | | |
| | Mutant Primer Sequence:- | | |
| | GGTTGAGGAAGGGGCTAGGACGGCTCCT | | |
| | GG (SEQ ID 52) | | |
| K143R | Wild Type Primer Sequence:- | AAG→AGA | No silent mutation |
| | AGGCGCTAAGACGGCTCCTGGAAAGAA | | |
| | GAGACCGGTAGAGCCA (SEQ ID 122) | | |
| | Mutant Primer Sequence:- | | |
| | AGGCGCTAAGACGGCTCCTGGAAAGAG | | |
| | AAGACCGGTAGAGCCA (SEQ ID 53) | | |
| K652R | Wild Type Primer Sequence:- | AAG→CGA | C→T disappearance of |
| | TGGCCTGAAACATCCTCCGCCTCAGATC | | BclI, DpnI, Hpy188I |
| | CTGATCAAGAACACGCCTGTACCTGCGG | | |
| | (SEQ ID 123) | | |
| | Mutant Primer Sequence:- | | |
| | TGGCCTGAAACATCCTCCGCCTCAGATC | | |
| | CTGATTCGAAACACGCCTGTACCTGCGG | | |
| | (SEQ ID 54) | | |

TABLE 11
describes the details of primers used for site-directed mutagenesis of specific
Serine/threonine to Alanine and Lysine to Arginine residues in AAV9 capsid

| | | | CHANGE IN |
|---|---|---|---|
| | | | RESTRICTION |
| | | NUCLEOTIDE | ENZYME DUE TO |
| RESIDUE | SEQUENCE (5′ to 3′) | CHANGE | SILENT MUTATION |
| AAV9- | Wild Type Primer Sequence:- | AGC→GCA | No silent mutation |
| S278A | CAACGCCTACTTCGGCTACAGCACCCCCTGGGG | | |
| | GTATTTTG (SEQ ID 124) | | |
| | Mutant Primer Sequence:- | | |
| | CAACGCCTACTTCGGCTACGCAACCCCCTGGGG | | |
| | GTATTTTG (SEQ ID 55) | | |
| AAV9- | Wild Type Primer Sequence:- | TCA→GCG | No silent mutation |
| S490A | CAGCTACCGACAACAACGTGTCTCAACCACTGT | | |
| | GACTCAAAACAACA (SEQ ID 125) | | |
| | Mutant Primer Sequence:- | | |
| | CAGCTACCGACAACAACGTGTCGCGACCACTGT | | |
| | GACTCAAAACAACA (SEQ ID 56) | | |
| AAV9- | Wild Type Primer Sequence:- | TCT→GCC | C→T HypAV |
| S669A | GCCTTCAACAAGGACAAGCTGAACTCTTTCATC | | disappears |
| | ACCCAGTATTCTACTGGCCAA (SEQ ID 126) | | |
| | Mutant Primer Sequence:- | | |
| | GCCTTCAACAAGGACAAGCTGAACGCCTTTATC | | |
| | ACCCAGTATTCTACTGGCCAA (SEQ ID 57) | | |
| AAV9- | Wild Type Primer Sequence:- | AGC→GCC | A→G Tsp509I, |
| S499A | CCACTGTGACTCAAAACAACAACAGCGAATTTG | | ApoI disappears |
| | CTTGGCCTGGAGCTTC (SEQ ID 127) | | |
| | Mutant Primer Sequence:- | | |
| | CCACTGTGACTCAAAACAACAACGCCGAGTTTG | | |
| | CTTGGCCTGGAGCTTC (SEQ ID 58) | | |
| AAV9- | Wild Type Primer Sequence:- | ACC→GCC | C→A AciI |
| T251A | CCCGAACCTGGGCCCTGCCCACCTACAACAATC | | disappears |
| | ACCTCTA (SEQ ID 128) | | |
| | Mutant Primer Sequence:- | | |
| | CCCGAACCTGGGCCCTGCCAGCCTACAACAATC | | |
| | ACCTCTA (SEQ ID 59) | | |
| AAV9- | Wild Type Primer Sequence:- | AAG→AGA | MboII disappears |
| K143R | AGCGGCTAAGACGGCTCCTGGAAAGAAGAGGC | | |
| | CTGTAGAGCAG (SEQ ID 129) | | |
| | Mutant Primer Sequence:- | | |
| | AGCGGCTAAGACGGCTCCTGGAAAGAGAAGGC | | |
| | CTGTAGAGCAG (SEQ ID 60) | | |

TABLE 12
describes the details of primers used for site-directed mutagenesis
of specific Serine/Threonine to Alanine and Lysine to Arginine
residues in AAVrh.10

| | | NUCLEOTIDE |
|---|---|---|
| RESIDUE | SEQUENCE (5′ to 3′) | CHANGE |
| K84R | Wild Type Primer Sequence:- | AAA→AGA |
| | GCCTACGACCAGCAGCTCAAAGCGGGTGAC (SEQ ID 130) | |
| | Mutant Primer Sequence:- | |
| | GCCTACGACCAGCAGCTCAGAGCGGGTGAC (SEQ ID 61) | |
| K137R | Wild Type Primer Sequence:- | AAG→AGG |
| | TGGTTGAGGAAGGCGCTAAGACGGCTCCT (SEQ ID 131) | |
| | Mutant Primer Sequence:- | |
| | TGGTTGAGGAAGGCGCTAGGACGGCTCCT (SEQ ID 62) | |
| K333R | Wild Type Primer Sequence:- | AAG→AGG |
| | GCAGAATGAAGGCACCAAGACCATCGCCAATAACC (SEQ ID | |
| | 132) | |
| | Mutant Primer Sequence:- | |
| | GCAGAATGAAGGCACCAGGACCATCGCCAATAACC (SEQ ID | |
| | 63) | |
| S492A | Wild Type Primer Sequence:- | TCC→GCC |
| | GCAGCAACGCGTCTCCACGACACTGTC (SEQ ID 133) | |
| | Mutant Primer Sequence:- | |
| | GCAGCAACGCGTCGCCACGACACTGTC (SEQ ID 64) | |
| S501A | Wild Type Primer Sequence:- | AGC→GCC |
| | CTGTCGCAAAATAACAACAGCAACTTTGCCTGGACCGG (SEQ | |
| | ID 134) | |
| | Mutant Primer Sequence:- | |
| | CTGTCGCAAAATAACAACGCCAACTTTGCCTGGACCGG (SEQ | |
| | ID 65) | |
| S671A | Wild Type Primer Sequence:- | TCG→GCG |
| | CAAGCTAAGCTGGCGTCGTTCATCACGCAGT (SEQ ID 135) | |
| | Mutant Primer Sequence:- | |
| | CAAGCTAAGCTGGCGGCGTTCATCACGCAGT (SEQ ID 66) | |
| T108A | Wild Type Primer Sequence:- | AGC→GCGH |
| | GCGTCTGCAAGAAGATACGTCTTTTGGGGGC (SEQ ID 136) | |
| | Mutant Primer Sequence:- | |
| | GCGTCTGCAAGAAGATGCGTCTTTTGGGGGC (SEQ ID 67) | |
| T252A | Wild Type Primer Sequence:- | ACC→GCC |
| | CTGGGCCCTCCCCACCTACAACAACCA (SEQ ID 137) | |
| | Mutant Primer Sequence:- | |
| | CTGGGCCCTCCCCGCCTACAACAACCA (SEQ ID 68) | |
| T674A | Wild Type Primer Sequence:- | ACG→GCG |
| | TGGCGTCGTTCATCACGCAGTACAGCACC (SEQ ID 138) | |
| | Mutant Primer Sequence:- | |
| | TGGCGTCGTTCATCGCGCAGTACAGCACC (SEQ ID 69) | |

In an embodiment, the aim of the present invention is to arrive at AAV vectors having mutations in their capsid protein. Such mutated AAV vectors comprise mutations in either Serine, Threonine or Lysine amino acids, or any combination of mutations thereof. Further, within a AAV vector sequence, such mutations may occur at one or multiple places and any combination of such mutations fall within the purview of this invention. Sequences provided by SEQ ID Nos. 148 to 157 only represent examples of such vector sequences having all the mutations in each serotype, and should not be construed to limit the instant invention to only these sequences. The aim of the invention is to cover AAV vector sequences which may comprise either one, either few or either all of the mutations provided by SEQ ID Nos. 148 to 157.

The disclosure is further illustrated by the following Examples. The following examples are included to demonstrate preferred embodiments of the disclosure. It should be appreciated by those of skill in the art that the techniques disclosed in the examples which follow represent techniques discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. The present disclosure is not to be limited in scope by the specific embodiments described herein, which are intended as illustrations of particular aspects of the disclosure, and functionally equivalent methods and components are within the scope of the disclosure. Indeed, various modifications of the disclosure, in addition to those shown and described herein, will become apparent to those skilled in the art from the foregoing description. Such modifications are intended to fall within the scope of the appended claims.

### Examples

### Structural Analysis of AAV Capsid

### Structural Analysis of AAV2 Capsid:

The three-dimensional structure of the AAV2 capsid from the protein Data bank (PDB accession number 1LP3) is analyzed extensively. Protein-protein interaction interface residues on the capsid proteins are determined by accessibility-based method. Solvent accessibility values of the residues are determined with the NACCESS program. Phosphorylation sites in capsid protein are predicted with NetPhosK, kinasePhos and Scansite, prediction of k-spaced Amino Acid Pairs and prediction of Ubiquitination sites with Bayesian discriminant Method.

Structures are visualized with the PyMOL software package. To assess the conservation of the predicted phosphorylation as well as ubiquitination sites, multiple sequence alignment of the VP1 sequence across the 10 serotypes is generated with ClustalW.

### Structural Analysis of AAV8 Capsid:

The three dimensional structure of the AAV8 capsid (PDB code-2qa0) is analyzed extensively to determine interaction interfaces of capsid protein chains. Accessibility-based method is employed to determine the residues participating in the protein-protein interactions between capsid proteins. Solvent accessibility of every residue is computed using NACCESS tool and the residues are grouped as solvent-exposed if the solvent accessibility values are more than 7%, while those with lesser accessibility are called buries residues. The residues are called interface residues if they are buried (accessibility <7%) in protein complex while being solvent-exposed (accessibility 0.10%) in isolated chains.

The structure of the viral capsid is visualized using PYMOL software and compared to the structure of AAV2 capsid using DALIlite tool for structure-based comparison.

The above mentioned structural analysis is similarly performed for other serotypes (AAV1, AAV3-AAV7 and AAV9-AAV10). From the above explanation the person skilled in the art will be able to perform the structural analysis for AAV serotypes without any undue experimentation.

### Methodology to Arrive at Ser/Thr/Lys Mutants Across all Serotypes by Site-Directed Mutagenesis:

Site directed mutagenesis is performed on wild type rep-cap plasmid pACG2/R2c by Quik Change II XL Site-Directed Mutagenesis Kit (Stratagene, Calif., USA) as per the manufacturer's protocol. Briefly, a one step PCR is performed for 18 cycles with the mutation containing primers followed by DpnI digestion for 1 h. Primers are designed to introduce amino acid change from serine/threonine to alanine or lysine to arginine (refer table 4). Transformation of XL10-Gold Ultracompetent Cells is carried out with 2 μL of DPN1 digested DNA followed by plating in agar plates containing ampicillin according to the manufacturer's protocol (Stratagene). Plasmids isolated from colonies are confirmed for the presence of the desired point mutation by restriction digestion and DNA sequencing, prior to using them for packaging viral vectors.

### Production of Recombinant AAV2 Vectors:

Highly purified stocks of self-complementary (sc) AAV2-WT or 26 capsid mutants of AAV2 vectors or AAV8-WT vector carrying the enhanced green fluorescent protein (EGFP) gene driven by the chicken b-actin promoter are generated by polyethyleneimine-based triple transfection of AAV-293 cells. Briefly, forty 150-mm² dishes 80% confluent with AAV-293 cells are transfected with AAV2 rep/cap (p.ACG2), transgene (dsAAV2-EGFP), and AAV-helper free (p.helper) plasmids. Cells are collected 72 hr post transfection, lysed, and treated with Benzonase nuclease (25 units/ml; Sigma-Aldrich). Subsequently, the vectors are purified by iodixanol gradient ultracentrifugation (OptiPrep; Sigma-Aldrich) followed by column chromatography (HiTrap SP column; GE Healthcare Life Sciences, Pittsburgh, Pa.). The vectors are finally concentrated to a final volume of 0.5 ml in phosphate-buffered saline (PBS), using Amicon Ultra 10K centrifugal filters (Millipore, Bedford, Mass.). The physical particle titers of the vectors are quantified by slot-blot analysis and expressed as vector genomes per milliliter.

### Production of Recombinant AAV8 Vectors:

Highly purified stocks of self-complementary wild-type (WT) AAV8, or the mutant AAV8 vectors encoding the enhanced green fluorescent protein (EGFP) gene driven by the chicken b-actin promoter containing the cytomegalovirus (CMV) enhancer and SV40 poly A signal or the human coagulation factor IX (h.FIX) under the control of liver-specific promoters, human alpha-1-antitrypsin (hAAT) or LP1 promoter (consisting of core liver specific elements from human apolipoprotein hepatic control region) are generated by polyethyleneimine-based triple transfection of AAV-293 cells (Stratagene). Briefly, forty numbers of 150-mm² dishes 80% confluent with AAV 293 cells are transfected with AAV8 rep-cap, transgene-containing and AAV-helper free (p.helper) plasmids. Cells are collected 72 hr post-transfection, lysed, and treated with 25 units/ml of benzonase nuclease (Sigma Aldrich, St Louis, Mo.). Subsequently, the vectors are purified by iodixanol gradient ultracentrifugation (Optiprep, Sigma Aldrich) followed by column chromatography (HiTrap Q column, GE Healthcare, Pittsburgh, Pa.). The vectors are finally concentrated to a final volume of 0.5 ml in phosphate buffered saline (PBS) using Amicon Ultra 10K centrifugal filters (Millipore, Bedford, Mass.). The physical particle titers of the vectors are quantified by slot blot analysis and expressed as viral genomes (vgs)/ml.

### Recombinant AAV Vector Transduction Assays In Vitro

To assess the effect of pharmacological inhibition of cellular serine/threonine kinases on AAV transduction, approximately 1.6×10⁵ HeLa cells are mock (PBS)-treated or pretreated with optimal concentrations of PKA inhibitor (25 nM), PKC inhibitor (70 nM), or CKII inhibitor (1 lM), or with a combination of each of these inhibitors overnight and transduced with AAV-WT vector at 2×10³ VG/cell. The safe and effective concentration of kinase inhibitors used is determined by 3-(4,5-dimethylthiazol-2-yl)-2,5-diphenyltetrazolium bromide (MTT) assay, performed with three 10-fold dilutions around the median inhibition constant (IC₅₀) values for these small-molecule inhibitors. Twenty-four hours later, transgene expression is measured by flow cytometry (FACS Calibur; BD Biosciences, San Jose, Calif.). A total of 1×10⁴ events are analyzed for each sample. Mean values of percent EGFP positivity from three replicate samples are used for comparison between treatment groups. To assess the efficacy of the novel mutant vectors generated, HeLa or HEK-293 cells are mock-infected or infected with either AAV-WT or AAV S/T/K mutant vector (2103 VG/cell). Forty-eight hours post-transduction, transgene expression is quantitated by flow cytometry (FACSCalibur; BD Biosciences) or captured by EGFP imaging. For flow cytometric analysis, HeLa or HEK-293 cells are trypsinized (0.05% trypsin; Sigma-Aldrich) and rinsed twice with PBS (pH 7.4). A total of 1×10⁴ events are analyzed for each sample. In total, three independent experiments are performed including three intraassay replicates in each of the experiment. Mean values of percent GFP positivity from these nine replicate samples are used for comparison between AAV-WT- and AAV S/T/K-infected cells.

### Result and Conclusion:

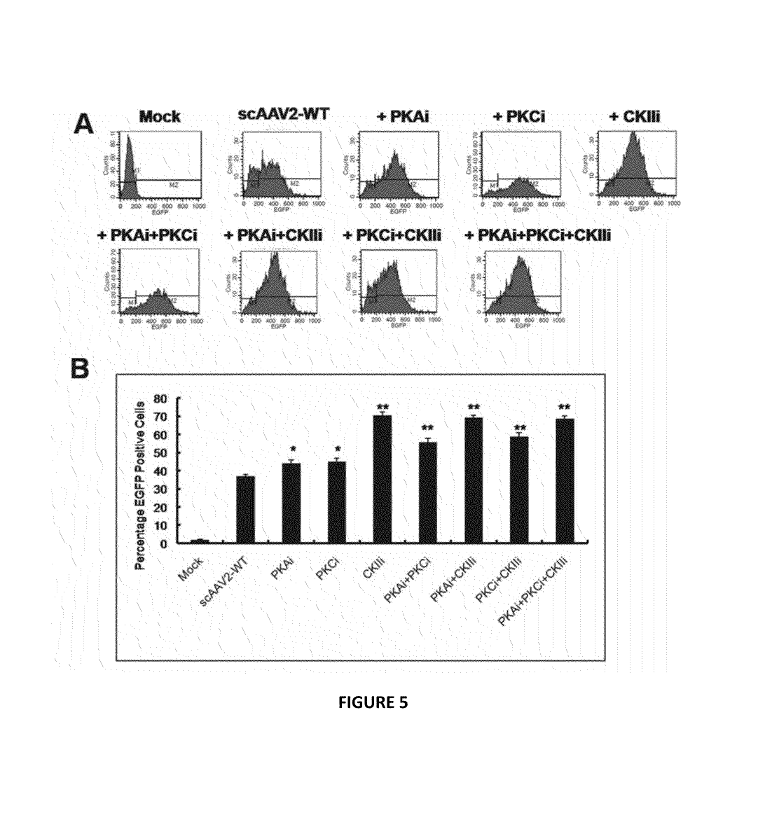

In silico analysis of the AAV2 capsid structure using various phosphorylation prediction tools identified PKA, PKC and CKII kinases as major binding partners of phosphodegrons of AAV2 capsid. Since these enzymes are primarily serine/threonine kinases with an ability to phosphorylate S/T residues, the kinase activity is inhibited by specific small molecule inhibitors and then infected the HeLa cells with scAAV2-EGFP vector. As described in FIG. 5A/B, a significantly higher gene expression of the AAV2-WT vector is observed when HeLa cells are pre-treated with these kinase inhibitors, with a maximal 90% increase seen in cells treated with the CKII inhibitor. This demonstrates that one or more surface-exposed serine and/or threonine amino acid in the AAV2 capsid is phosphorylated within the host cell by PKA, PKC and CKII serine/threonine kinases and specific inhibition of this process improves the gene expression from AAV vectors. Since, systemic administration of serine/threonine kinase inhibitors in an in vivo setting is likely to be toxic (Force and Kolaja, 2011), we instead chose to modify the kinase target substrates in the AAV2 capsid to further improve the transduction efficiency of AAV2 vectors with negligible or no toxicity/side-effects. The transduction potential of nine single-mutant and two double mutant AAV2 K→R vectors in HeLa cells at an MOI of 2000. The K532R and K544R single mutants and one double mutant (K490+532R) showed transduction efficacy of about 70% to about 82% when compared 30% efficacy in AAV2-WT vector. Similar results are observed for the mutant vectors—T251A, S276A, S489A, S498A, and K532R in HEK-293 cells.

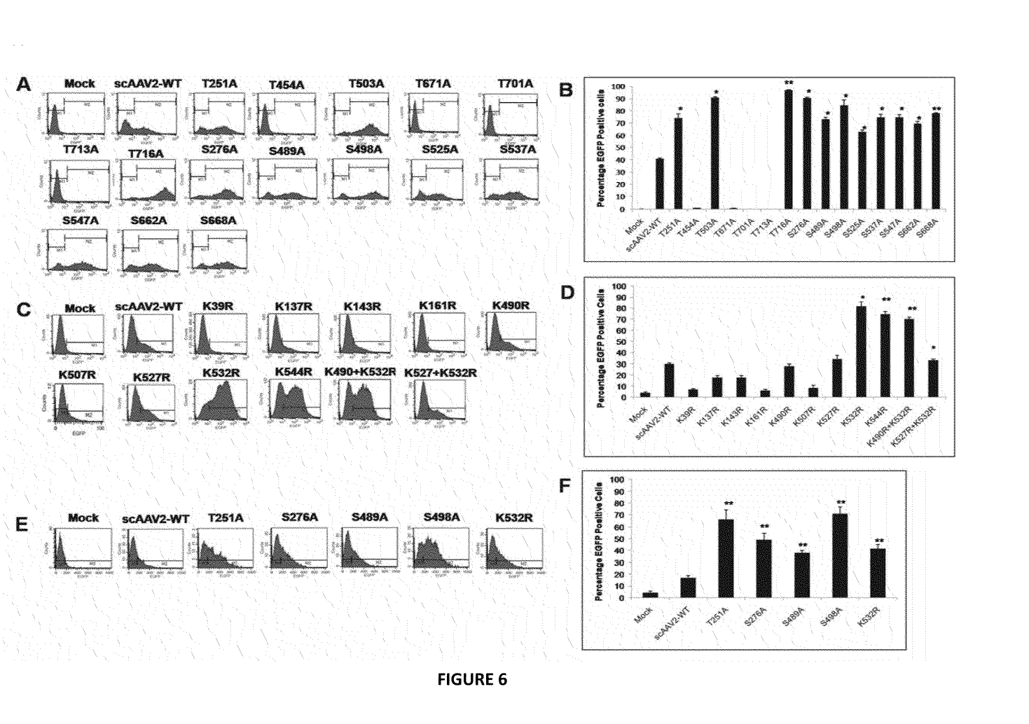

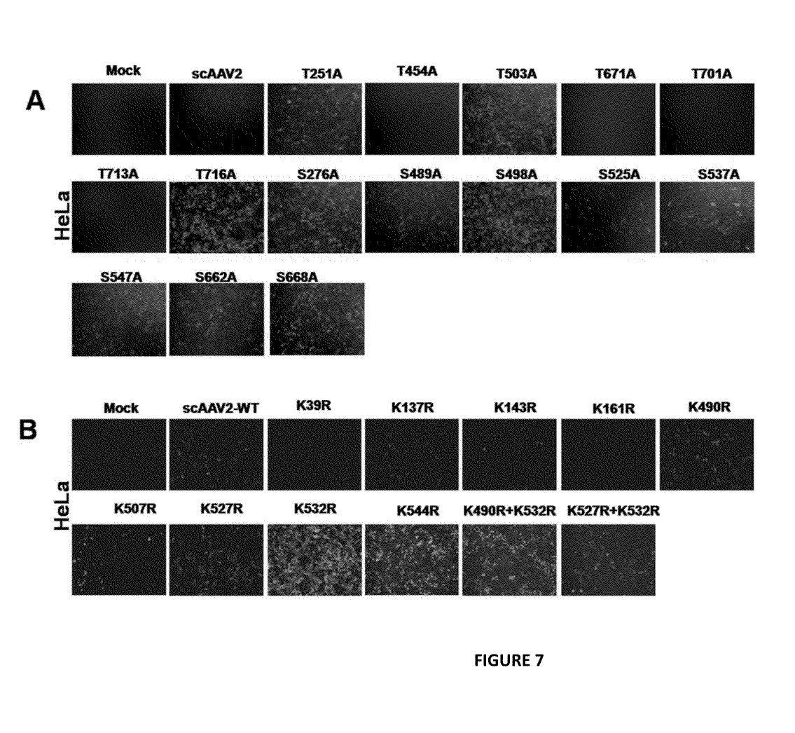

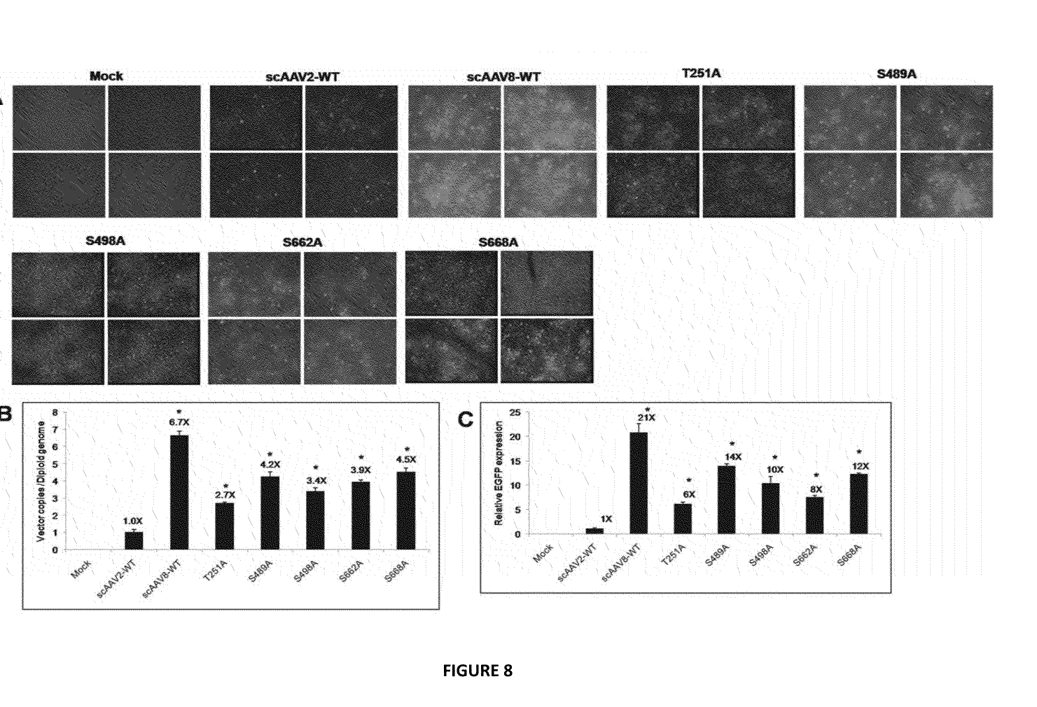

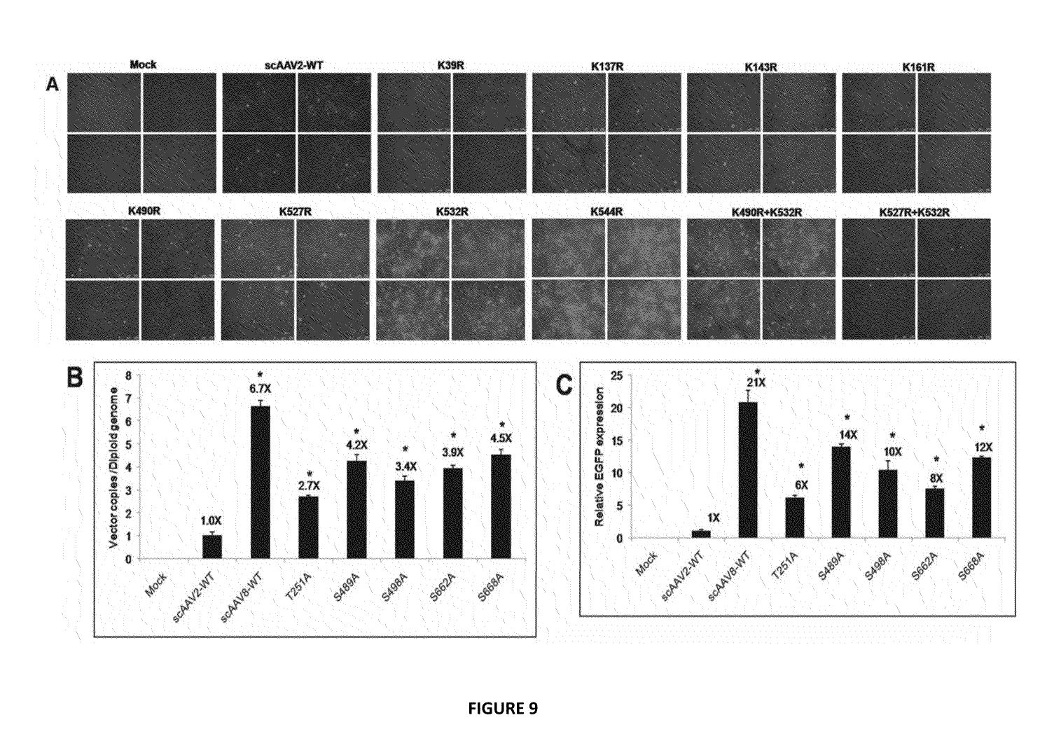

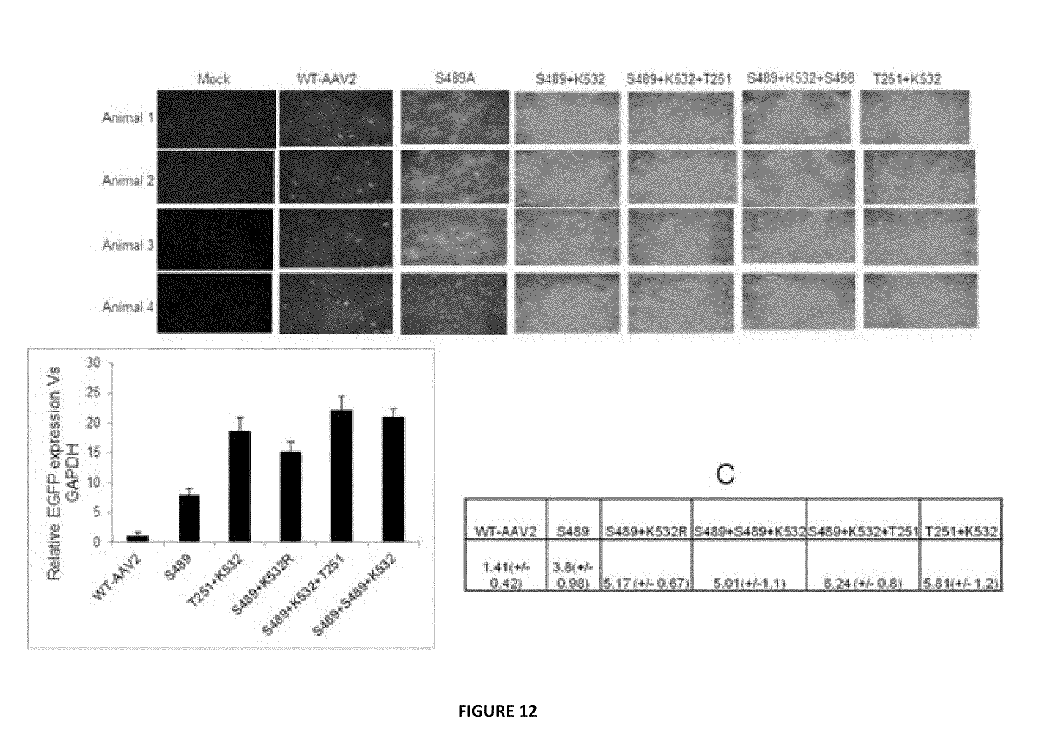

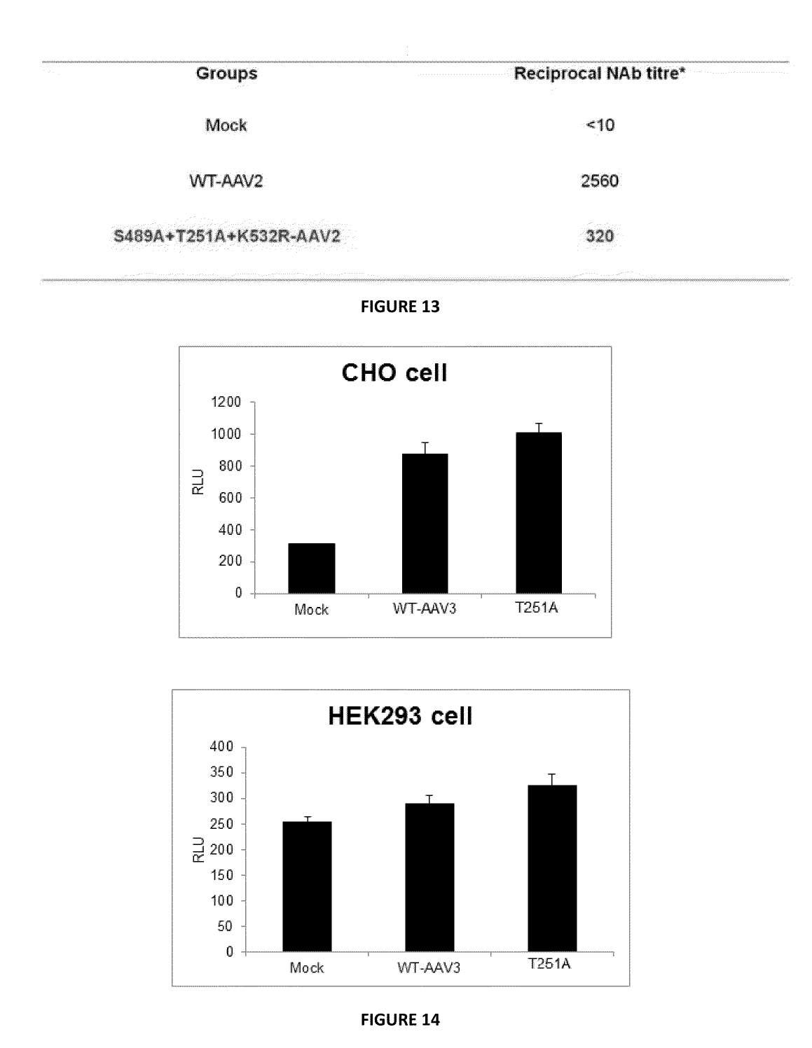

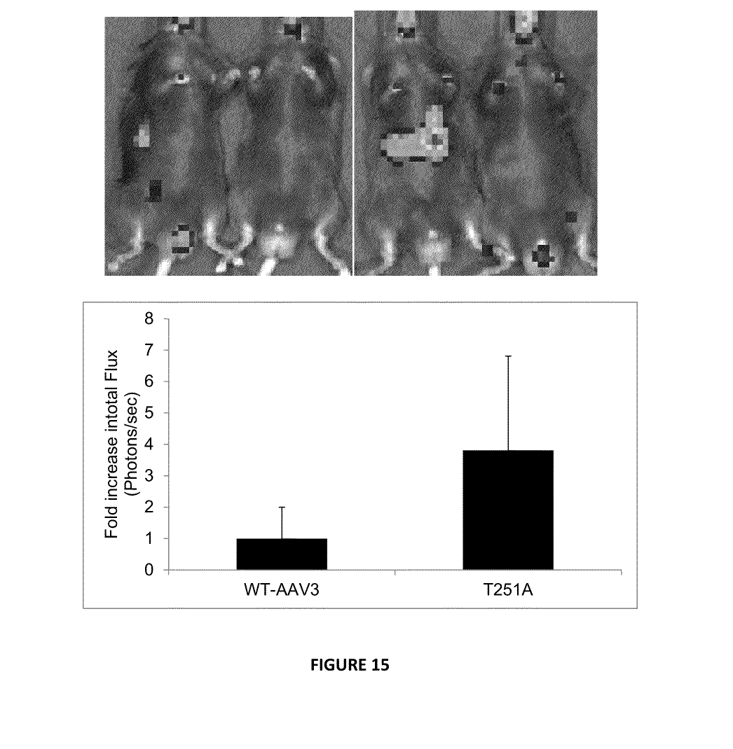

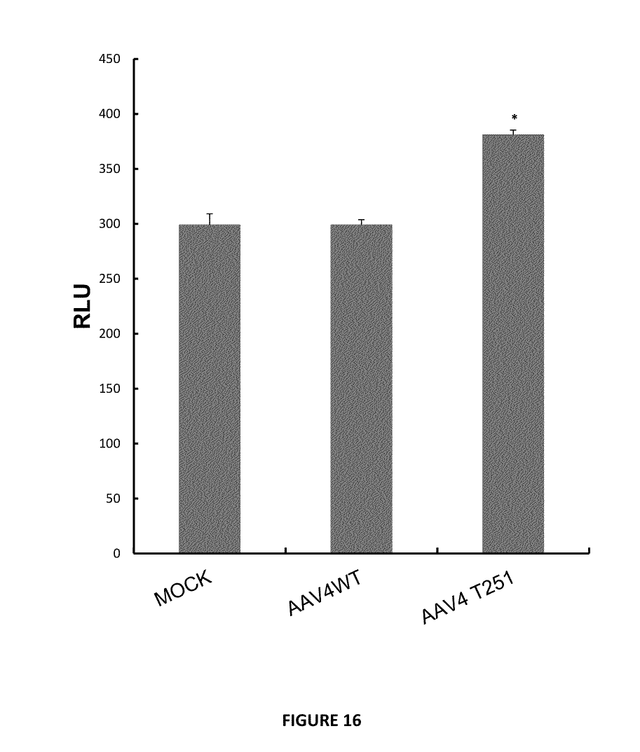

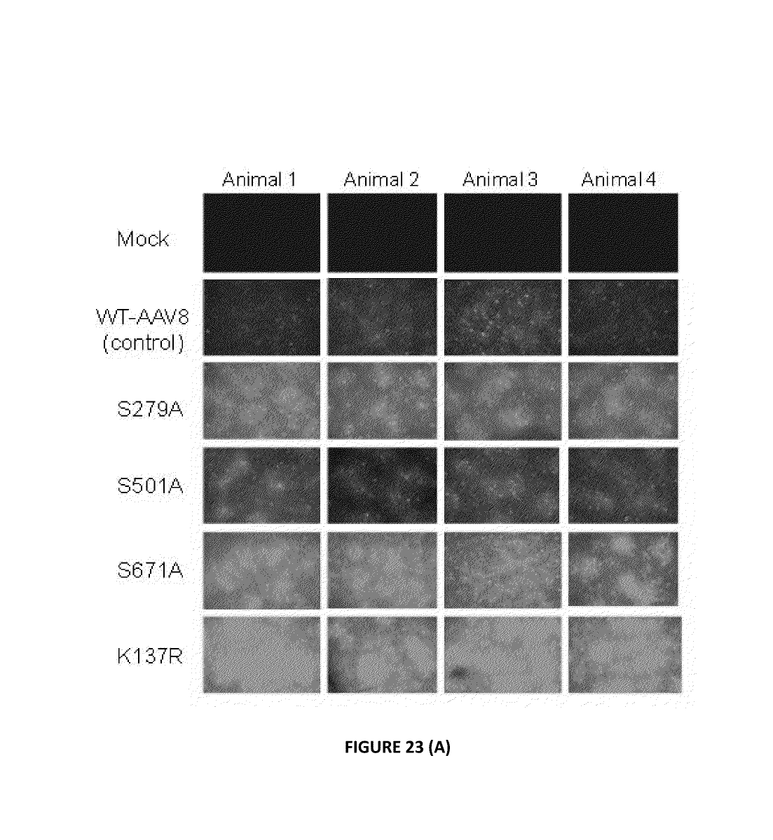

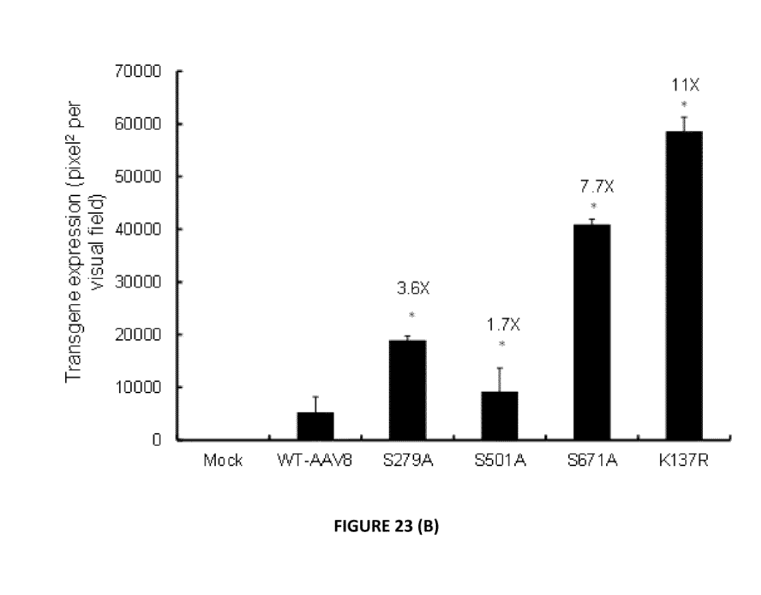

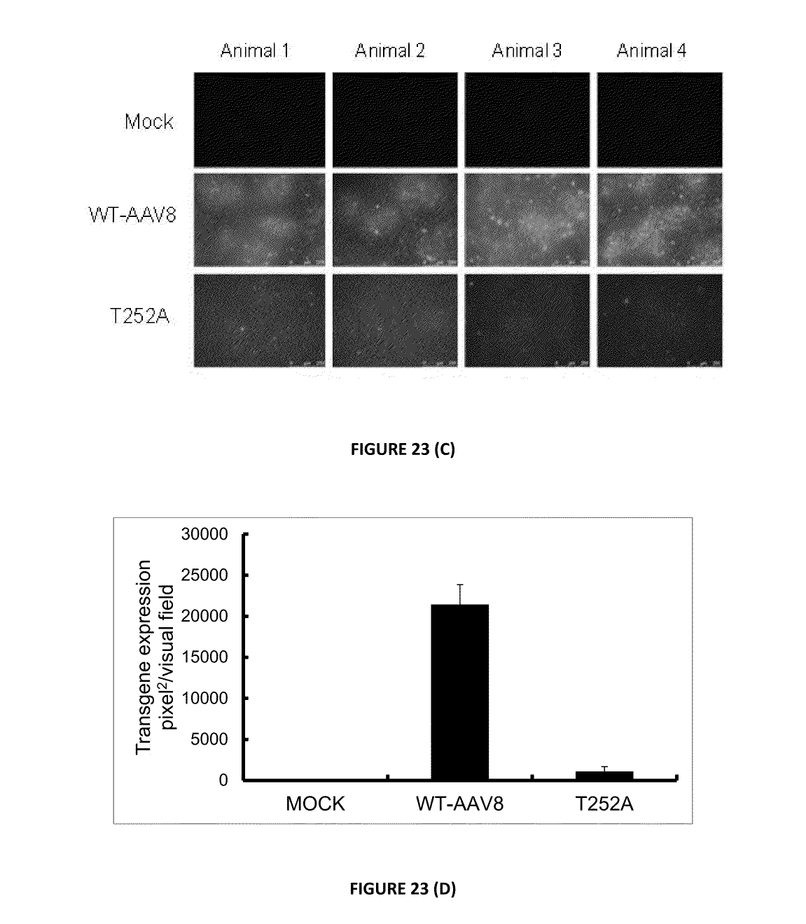

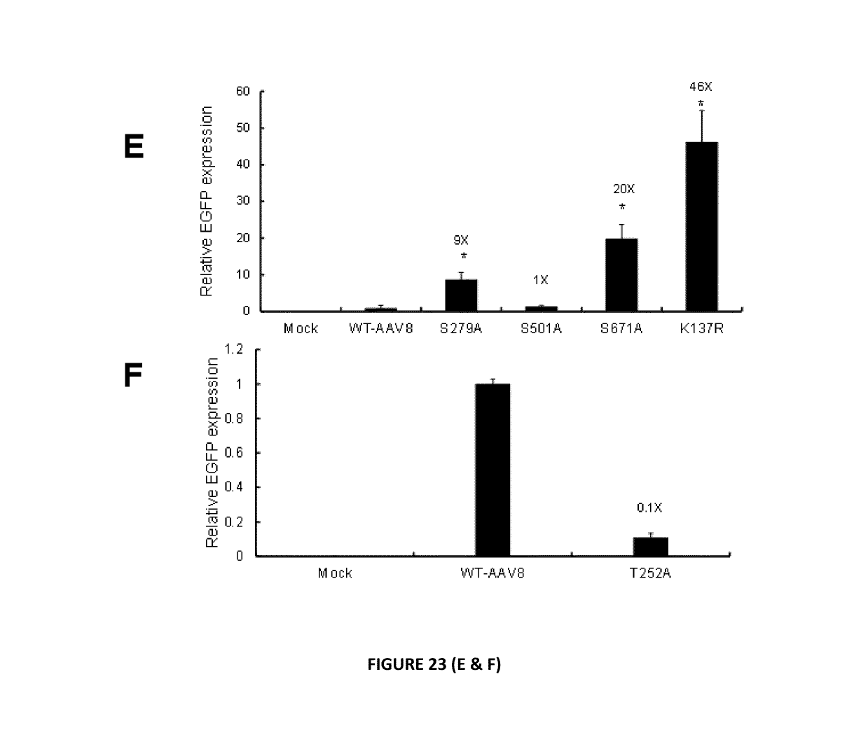

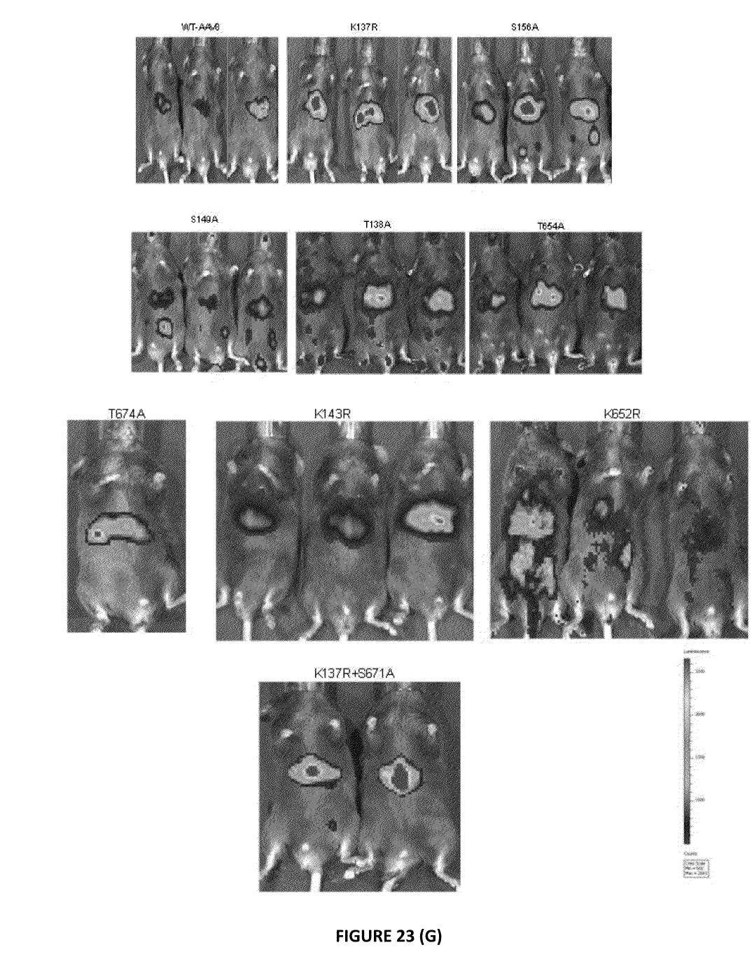

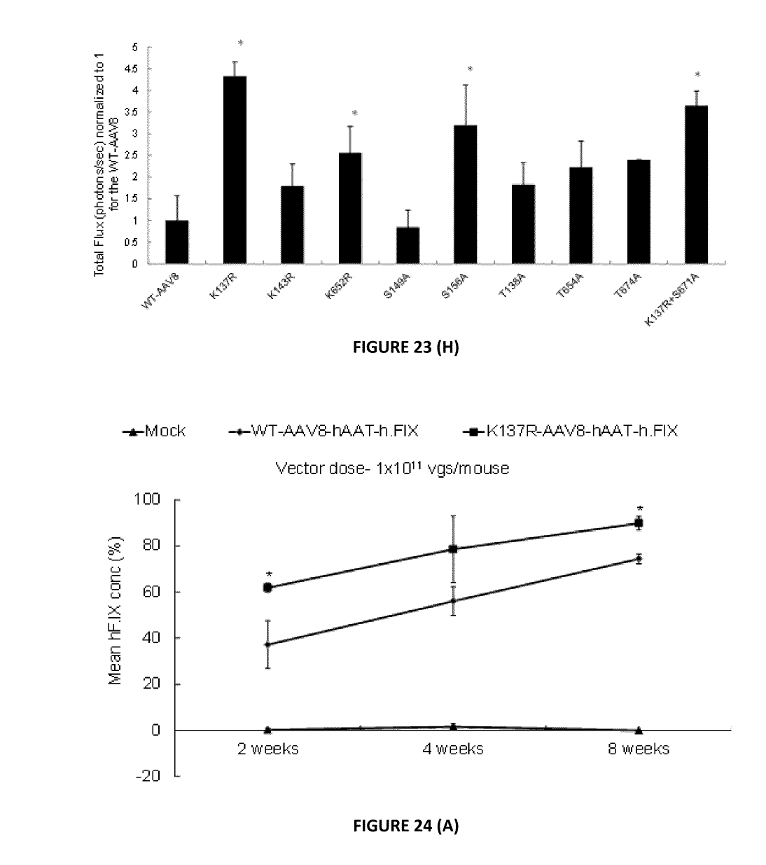

A mutant is deduced to have an improved transduction profile, if it shows an increase in GFP gene expression over and above the wild-type AAV vectors, by either FACS analysis or by fluorescence imaging. FIGS. 5, 6 and 17 A demonstrates a significant increase in EGFP gene expression for the various Ser/Thr/Lys mutants tested by either FACS or by fluorescence microscopy. A maximal increase can be seen in vitro with S668A (7 fold), T251A (5.5 fold) or K532R (13.5 fold) mutants. This increase is further shown qualitatively for the Ser/Thr>Ala mutants as illustrated in FIGS. 7A, 8, or Lys>Arg mutants as illustrated in FIGS. 7B, 9 in HeLa cells by fluorescence imaging. Further, FIG. 12 illustrates the fluorescence imaging of single and double or triple mutant vectors in AAV2, while FIG. 23G/H shows luciferase imaging of AAV8 double mutant vector.

### Recombinant AAV Vector Transduction Studies In Vivo

C57BL/6 mice are purchased from Jackson Laboratory (Bar Harbor, Me.). All animal experiments are approved and carried out according to the institutional guidelines for animal care (Christian Medical College, Vellore, India). Groups (n=4 per group) of 8 to 12 week old C57BL/6 mice are mock-injected or injected with 5×10¹⁰ VG each of scAAV-WT or scAAV S/T/K mutant vector carrying the EGFP transgene, via the tail vein. Mice are euthanized 4 weeks after vector administration. Cross-sections from three hepatic lobes of the mock-injected and vector-injected groups are assessed for EGFP expression by fluorescence microscopy.

### Estimation of AAV Vector Genome Copies and EGFP Expression in Murine Hepatocytes by Quantitative PCR Analysis:

Groups of 8- to 12-week-old C57BL/6 mice (n=4-8 per group) are mock-injected or injected with 5×10¹⁰ vgs each of WT-AAV or AAV mutant vectors, containing EGFP as the transgene, via the tail vein. Mice are euthanized 2 or 4 weeks after vector administration. Cross-sections from three hepatic lobes of the mock-injected and vector-injected groups are assessed for EGFP expression by a fluorescence microscope (Leica CTR6000; GmbH, Stuttgart, Germany). Images from five visual fields of mock-infected and vector infected cells are analyzed by ImageJ analysis software (NIH, Bethesda, Md.). Transgene expression (mean value) is assessed as total area of green fluorescence and expressed as mean pixels per visual field (mean−SD). The best performing AAV capsid mutant, along with the WT AAV vector containing h.FIX as the transgene (under LP1 and hAAT promoter), are administered into 8 to 12 week-old male C57BL/6 mice (n=3-4 per group) intravenously at different doses (2.5×10¹⁰ and 1×10″ vgs per mouse). Blood is collected retro-orbitally 2, 4, and 8 weeks post-vector administration. The antigenic activity of hF.IX (FIX:Ag) is measured using a commercial kit (Asserachrom, Diagnostica Stago, Asniers, France).

Result:

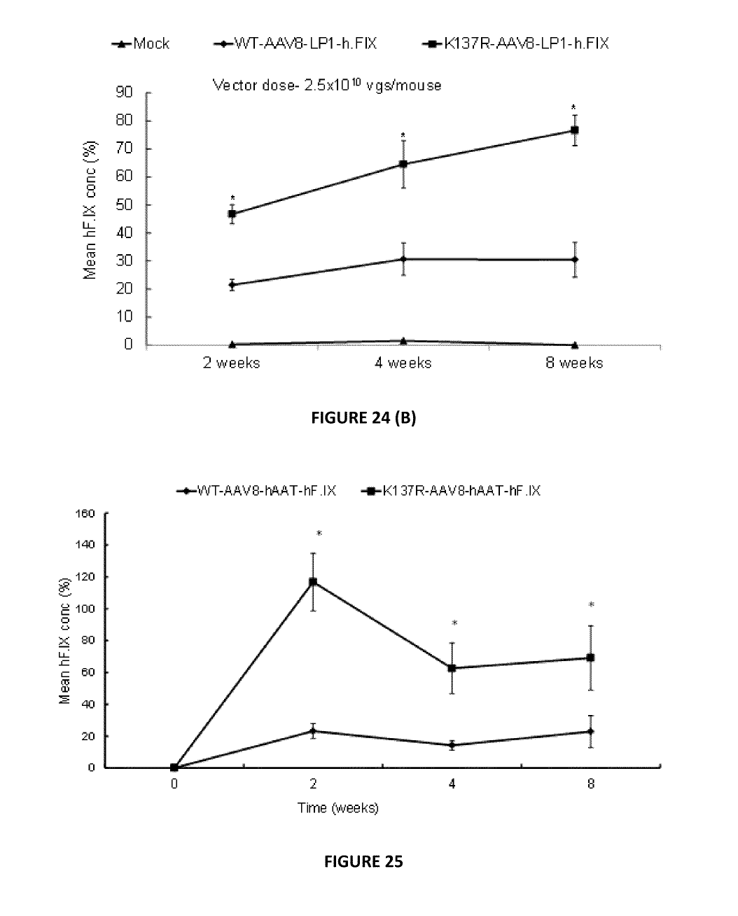

The circulating levels of h.FIX are higher in all the K137R AAV8-treated groups as compared to the WT-AAV8-treated groups either at 2 weeks (62% vs. 37% for hAAT constructs and 47% vs. 21% for LP1 constructs), 4 weeks (78% vs. 56% for hAAT constructs and 64% vs. 30% for LP1 constructs) or 8 weeks (90% vs. 74% for hAAT constructs and 77% vs. 31% for LP1 constructs) post-hepatic gene transfer. These expression levels further corroborate the potential of the K137R mutant for hepatic gene therapy of hemophilia B The K137R mutant has an increased level of h.FIX expression for up to 2 months post hepatic gene transfer as described in FIGS. 24 and 25.

### Biodistribution Studies:

Liver, spleen, lung, heart, kidney, and muscle tissue are collected from each of the mice administered with either WT-AAV or AAV S/T/K mutant vectors, 2 or 4 weeks after gene transfer. Genomic DNA is isolated using the QIAamp DNA Mini Kit (Qiagen, Valencia, Calif.) according to the manufacturer's protocol. Quantitative polymerase chain reaction (PCR) is carried out to estimate the vector copy numbers in 100 ng of template genomic DNA by amplifying the viral inverted terminal repeats (ITRs) with specific probes/primers as described using a low ROX qPCR mastermix according to manufacturer's protocol (Eurogentec, Seraing, Belgium). Data is captured and normalized to mouse glyceraldehyde-3-phosphate dehydrogenase (GAPDH) housekeeping control gene and analyzed in an ABI Prism 7500 Sequence Detection System Version 1.1 Software (Life Technologies, Applied Biosystems).

### Real-Time PCR Assay:

Groups (n=4 per group) of 8- to 12-week-old C57BL/6 mice are mock-injected or injected with 1×10¹¹ vgs each of WT-AAV or AAV S/T/K mutant vectors intravenously. Two hours later, mice are euthanized. Total RNA is isolated from liver sections of each mouse using the NucleoSpin RNA isolation kit (Machery-Nagel, Du\ddot{} ren, Germany). Approximately 2 μg of RNA is reverse transcribed using the first-strand RT kit (Qiagen, SABiosciences). The transcript levels of interleukin (IL) 1; IL6; tumor necrosis factor (TNF) α; Kupffer cells (KC); regulated on activation, normal T cell expressed and secreted (RANTES); IL12; toll-like receptor (TLR) 2; and TLR9 is measured using primers given below with a PCR master mix (MESA GREEN Mastermix plus, Eurogentec).

| | | SEQ |
|---|---|---|
| | | ID |
| Primer Name | Oligo sequence (5′ to 3′) | No. |
| Mouse IL1 alpha-F | TTTCCACAGCCACGAAGCT | 159 |
| Mouse IL1 alpha-R | TGATGAAAGGGCTCCCAAGT | 160 |
| Mouse IL6-F | TGTTCTCTGGGAAATCGTGGA | 161 |
| Mouse IL6-R | TCAGAATTGCCATTCGACAAC | 162 |
| Mouse TNF alpha-F | CCAGAACTCCAGGCGGT | 163 |
| Mouse TNF alpha-R | TGGGAACTTCTCATCCCTTTG | 164 |
| Mouse KC-F | GCCTATCGCCAATGAGCTG | 165 |
| Mouse KC-R | TTCGGTTTGGGTGCAGTG | 166 |
| Mouse RANTES-F | CTTTGCCTACCTCTCCCTCG | 167 |
| Mouse RANTES-R | CACACTTGGCGGTTCCTTC | 168 |
| Mouse IL12 alpha-F | TGCCACCTACTCCCTTGGAT | 169 |
| Mouse IL12 alpha-R | GGCTGTTGGAACGCTGAC | 170 |
| Mouse TLR-2-F | CAGCTTAAAGGGCGGGTCAGAG | 171 |
| Mouse TLR-2-R | TGGAGACGCCAGCTCTGGCTCA | 172 |
| Mouse TLR-9-F | CCAGACGCTCTTCGAGAACC | 173 |
| Mouse TLR-9-R | GTTATAGAAGTGGCGGTTGT | 174 |

Total RNA is isolated from the murine liver samples 2 or 4 weeks post-vector administration (5×10¹⁰ vgs per mouse) using TRIZOL_reagent (Sigma Aldrich) to measure EGFP transcript levels. Approximately 1 μg of RNA is reverse-transcribed using Verso™ Reverse Transcriptase according to the manufacturer's protocol (Thermo Scientific, Surrey, United Kingdom). TAQMAN_PCR is carried out using primers/probe against EGFP gene (Forward Primer: CTTCAAGATCCGCCACAACATC; Reverse Primer: ACC ATGTGATCGCGCTTCTC; Probe: FAM-CGCCGACCACTACCAGCAGAACACC-TAMRA) and according to the manufacturer's protocol (Eurogentec). GAPDH is used as the housekeeping control gene. Data is captured and analysed using the ABI Prism 7500 Sequence Detection (Life Technologies, Applied Biosystems).

Result:

Consistent with the in vitro studies, liver tissues of mice administered the four AAV2 S/A mutants (S489A, S498A, S662A, and S668A) and the T251A mutant showed higher levels of EGFP reporter when compared with animals injected with AAV2-WT vector and analyzed by fluorescence microscopy (FIG. 8A). A similar increase in EGFP levels is noted after hepatic gene transfer with the AAV2 lysine mutants K532R, K544R, and K490R+K532R, as described in FIG. 9A and further FIG. 12 describes serine/threonine/Lysine multiple-mutant vectors, K489R+K532R, S489+S489+K532, S489+K532+T251 and T251+K532 exhibiting enhanced transduction upon hepatic gene transfer in vivo. To confirm this phenomenon, we then measured AAV vector genome copy numbers in the liver tissue of vector- or mock-injected mice. As shown in FIGS. 8B and 9B, a significant increase in vector copies per diploid genome (up to 4.9-fold) is observed in animals injected with S/T/K mutant vectors in comparison with animals that received the AAV2-WT vector alone. To further corroborate these data, we then measured the transcript levels of EGFP in hepatic RNA isolated from these mice. The studies demonstrate higher levels of transgene transcript expression (up to 14-fold) after hepatic gene transfer, in AAV2 S/T/K mutant administered mice in comparison with AAV2-WT vector injected animals. In all these studies, AAV8-injected animals are used as a control group for hepatic gene transfer. From the above result it is very evident that the aforementioned data clearly suggests that select S/T→A and K→R mutations can augment the transduction efficiency of AAV2 vectors in vivo.

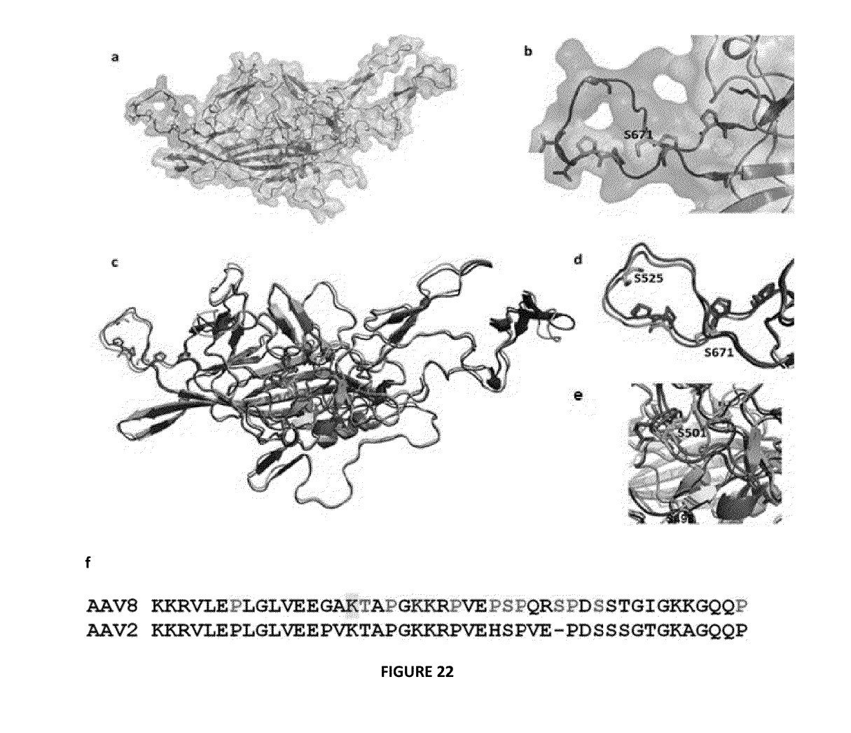

Two of the AAV8 S→A mutants (S279A and S671A) and the K137R mutant tested had a 3.6- to 11-fold higher EGFP expression by fluorescence imaging (FIGS. 23A and 23B) and a 9- to 46-fold higher EGFP transcript level as analyzed by quantitative PCR (FIG. 23E). The T252A mutant had lower levels of EGFP expression when compared to the WT-AAV8 vector, 2 weeks postadministration (FIGS. 23C, 23D, and 23F). When the transduction efficiency of the K137R mutant is assessed in a more immunogenic BALB/c strain of mice (Michou et al., 1997; Breous et al., 2010), a similar increase in transduction is noted for the K137R mutant (14-fold by fluorescence imaging, data not shown) as compared to the WT-AAV8 vector. To further corroborate this data, the biodistribution of AAV8 vectors in recipient mice is analysed by quantitative PCR measurement of vector genomes in various tissues. Table 13 demonstrates that the K137R mutant had the best tropism for liver as compared to the WT AAV8 (106 vs. 7.7 vector copies/mouse diploid genome). Interestingly, a preferential transduction of the lungs (0.13 vs. 0.03 vector copies/mouse diploid genome), and muscle tissue (1.38 vs. 0.45 vector copies/mouse diploid genome) is also noted for the K137R mutant suggesting that this vector has a better systemic transduction profile when compared to the other vectors tested. Similarly, the S/A mutant vectors (S279A, S501A, and S671A)\ generally showed increased transduction of the liver and muscle compared to WT-AAV8 vectors. However, the T252A mutant is considerably retargeted to other tissues such as the lungs, which may explain the low levels of EGFP expression seen in the liver (FIGS. 23C, 22D, and 22F). These results clearly indicate that the S/A and K/R mutant capsids augment the hepatic transduction of AAV8 vectors, with K137R being the most effective. Hence, this mutant in particular is subjected to further analysis to gain insights into mechanisms by which it exhibits its effects on transduction of the vector.

TABLE 13
Vector biodistribution in various organs in C57BL/6 mice 2-or 4-weeks post gene transfer with the WT-AAV8
and S/T/K mutant AAV8 vectors. Values are shown as mean (±SD) vector copy numbers per mouse diploid genome.

| Vector type | Muscle | Liver | Kidney | Lung | Spleen | Heart |
|---|---|---|---|---|---|---|
| WT-AAV8 | 0.45 ± 0.2 | 7.77 ± 0.8 | 0.0016 ± 0.00045 | 0.03 ± 0.07 | 0.004 ± 0.0009 | 0.0003 ± 0.00006 |
| (4 weeks) | | | | | | |
| K137R-AAV8 | 1.38 ± 0.4 | 106.8 ± 11 | 0.0011 ± 0.0003 | 0.13 ± 0.09 | 0.004 ± 0.0006 | 0.0006 ± 0.00004 |
| (4 weeks) | | | | | | |
| S279A-AAV8 | 2.84 ± 0.5 | 30.3 ± 3.2 | 0.0029 ± 0.0007 | 0.01 ± 0.03 | 0.002 ± 0.0005 | 0.0007 ± 0.00001 |
| (4 weeks) | | | | | | |
| S501A-AAV8 | 3.07 ± 0.6 | 15 ± 1.9 | 0.003 ± 0.0008 | 0.11 ± 0.08 | 0.0009 ± 0.0008 | 0.003 ± 0.0002 |
| (4 weeks) | | | | | | |
| S671A-AAV8 | 1.89 ± 0.3 | 65.89 ± 5.6 | 0.0006 ± 0.00012 | 0.051 ± 0.01 | 0.001 ± 0.0006 | 0.001 ± 0.0008 |
| (4 weeks) | | | | | | |
| WT-AAV8 | 0.008 ± 0.08 | 0.51 ± 0.1 | 0.0006 ± 0.0002 | 0.0008 ± 0.0002 | 0.022 ± 0.009 | 0.0001 ± 0.00005 |
| (2 weeks) | | | | | | |
| T252A | 0.01 ± 0.005 | 0.01 ± 0.002 | 0.0001 ± 0.00006 | 0.01 ± 0.004 | 0.0005 ± 0.00009 | 0.0006 ± 0.00009 |
| (2 weeks) | | | | | | |

### Estimation of Neutralizing Antibodies Against S/T/K AAV Vectors

Heat-inactivated serum samples from AAV-WT-injected or S→A and K→R mutant AAV injected C57BL/6 mice are assayed for neutralizing antibody (NAb) titers as described previously (Calcedo et al., 2009). Briefly, groups of mice (n=4) are administered 5×1010 VG of AAV-WT and AAV S/T/K mutant vectors via tail vein injections. Four weeks after vector delivery, animals are killed and serum is collected. The pooled serum is snap frozen to −80° C. Heat inactivated samples are assayed for the neutralizing antibody (NAb) titres as described previously (Calcedo et al., 2009). Dilutions of serum samples [1:5 to 1:81920] from animals are pre-incubated for 1 hr with AAV vectors and the mixture added to Huh7 cells. The NAb titer is reported as the highest plasma dilution that inhibited AAV transduction of Huh7 cells by 50% or more compared with that for the naive serum control. Limit of detection of the assay was 1/5 dilution. These values provide reciprocal titers of antibodies present in the sample analyzed i.e. higher the values of dilution that are required to cross-neutralize WT-AAV, the greater the concentration of antibodies that were originally present in the serum samples.

Result with AAV2:

AAV2 S489A vector demonstrates lower neutralization antibody titres compared to the WT-AAV2 vector. Pooled serum samples from WT-AAV2 or AAV2 mutant injected mice (n=4 per group) are analyzed for neutralizing antibodies 4-weeks after vector administration. Values are the reciprocal of the serum dilution at which relative luminescence units (RLUs) is reduced 50% compared to virus control wells (as described in the below table 14).

TABLE 14

| SL. No. | Groups | Reciprocal NAb Titre |
|---|---|---|
| 1 | scAAV2-WT | 5120 |
| 2 | S489A | 640 |
| 3 | S525A | 5120 |
| 4 | S537A | 5120 |
| 5 | S547A | 5120 |
| 6 | S662A | 5120 |
| 7 | Anti AAV2 Rabbit | 81920 |
| | Control Serum | |

Result with AAV8:

The mutant K137R vector is significantly less immunogenic when compared to WT-AAV8 vectors, which is demonstrated by measuring the neutralizing antibodies against the various mutants which demonstrated a 2-fold reduction in the neutralizing antibody titre for the K137R-AAV8 vector (as described in the below table 15).

TABLE 15

| S. No. | Groups | Reciprocal NAb titer |
|---|---|---|
| 1 | Mock | 0 |
| 2 | WT-AAV8 | 320 |
| 3 | S279A | 320 |
| 4 | S501A | 640 |
| 5 | S671A | 320 |
| 6 | K137R | 160 |
| 7 | Anti-AAV8 rabbit control | 20480 |
| | serum | |

### Recombinant AAV2 Vector Transduction Studies In Vivo

Groups (n=4, per group) of 8-12 weeks-old C57BL/6 are injected with ˜5×10¹⁰ viral genome particles (vg) of wild type (wt) scAAV2 or mutant scAAV2 vectors containing EGFP as the transgene, via the tail vein. Mock injection is done with PBS. Mice are euthanized 4 weeks after vector administration. Cross-sections from three hepatic lobes of the mock-injected and vector-injected groups assessed for EGFP expression by fluorescence microscopy. Images from five visual fields are analysed quantitatively by Adobe Photoshop CS2 software/Image-J and expressed as mean pixels per visual field (mean±SE).

Result:

In vivo studies are conducted in C57BL/6 mice, wherein ˜2 to 22 fold increase in transduction efficiency is observed upon hepatic gene transfer of ˜5×10¹⁰ vgs of AAV2 mutant vectors (Serine residues: S489A, S498A, S525A, S537A, S547A, S662A, S668A; threonine residue: T251A and lysine residue: K532R). The feasibility of the use of serine/threonine→alanine or lysine→arginine vectors either as single mutants (K532R, S668A, S662A, S489A, and T251A) or multiple mutants (K532R+S668A+S662A+S489A+T251A) for potential gene therapy of human diseases is illustrated in table 16 below.

TABLE 16
Transduction efficacy of S/T/K Mutants of AAV2

| | | In vitro Analysis: Fold | | | | |
|---|---|---|---|---|---|---|
| | | Change | | In vivo Analysis: Fold Change | | |
| | | Fluorescent | Flow | Fluorescent | EGFP expression | Vector copy |
| S. No. | AAV2 Mutants | Microscopy | Cytometry * | Microscopy | by QPCR | no by QPCR |

SINGLE MUTANTS

| | | | | | | |
|---|---|---|---|---|---|---|
| | AAV2 WT | 1 | 1.0 | 1 | 1 | 1 |
| 1 | AAV2 S276A | 5 | 2.3 | — | — | — |
| 2 | AAV2 S489A | 4.25 | 1.9 | 21.8 | 14 | 4.2 |
| 3 | AAV2 S498A | 5.8 | 2.1 | 4 | 10 | 3.4 |
| 4 | AAV2 S525A | 0.76 | 1.5 | 1.7 | — | — |
| 5 | AAV2 S537A | 0.65 | 1.8 | 7.3 | — | — |
| 6 | AAV2 S547A | 2.45 | 1.8 | 2.1 | — | — |
| 7 | AAV2 S662A | 1.7 | 1.7 | 17.4 | 8 | 3.9 |
| 8 | AAV2 S668A | 7.44 | 2.1 | 4 | 12 | 4.5 |
| 9 | AAV2 T251A | 5.54 | 1.9 | 4 | 6 | 2.7 |
| 10 | AAV2 T503A | 17 | 2.3 | | | |
| 11 | AAV2 T716A | 36 | 2.5 | | | |
| 12 | AAV2 K527R | 1.3 | 1.5 | 2 | 4.4 | 3.2 |
| 13 | AAV2 K532R | 18 | 2.7 | 4 | 11.8 | 4.5 |
| 14 | AAV2 K544R | 15 | 2.5 | 7 | 12.3 | 4.9 |

DOUBLE MUTANTS

| | | | | | | |
|---|---|---|---|---|---|---|
| 1 | AAV2 K490R + | 15 | 2.4 | 4 | 18 | 2.5 |
| | K532R | | | | | |
| 2 | AAV2 K527R + | 3 | 1.1 | 0.2 | 0.7 | 1.9 |
| | K532R | | | | | |
| 3 | AAV2 T251A + | — | 3.4 | — | 20 | 5.1 |
| | K532R | | | | | |
| 4 | AAV2 S489 + | — | 3.6 | — | 15 | 4.9 |
| | K532R | | | | | |

TRIPLE MUTANT

| | | | | | | |
|---|---|---|---|---|---|---|
| 1 | AAV2 T251A + | — | 3.4 | — | 22 | 6.2 |
| | K532R + S489A | | | | | |
| 2 | AAV2 S489A + | — | 3 | — | 22 | 5.8 |
| | S498A + K532R | | | | | |

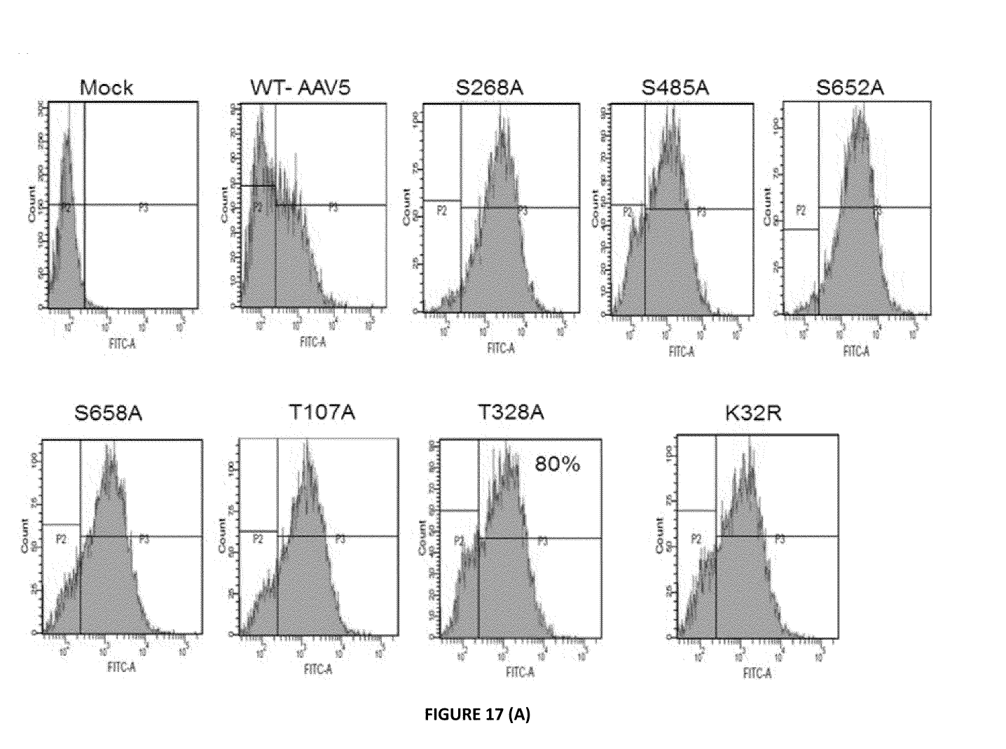

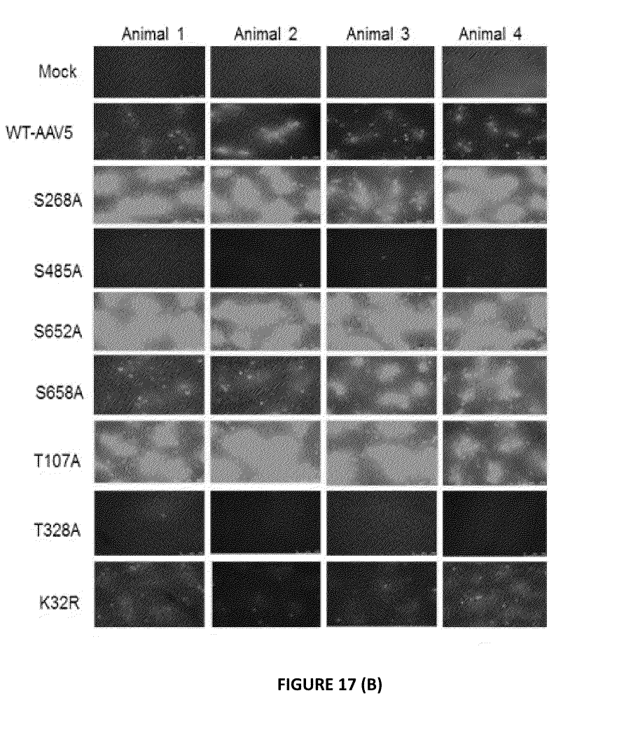

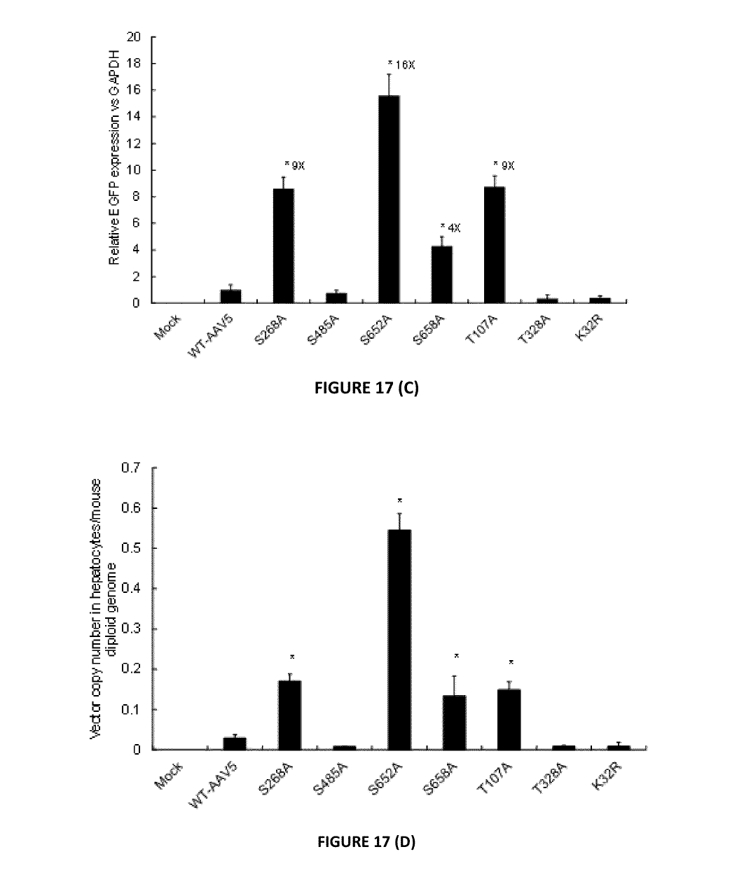

Further, quantitative analysis of mutant AAV2 and AAV8 depicting the transduction efficacy was also studied for the mutants mentioned herein. The data is provided in FIG. 17 below.

TABLE 17
Quantitative analysis of mutant AAV2 and AAV8 depicting
the transduction efficacy.

| | | In vivo Analysis: Fold Change | | |
|---|---|---|---|---|
| | | Vector copy | EGFP expression | Luciferase |
| S. No. | AAV8 Mutants | no by QPCR | by QPCR | expression |

SINGLE MUTANTS

| | | | | |
|---|---|---|---|---|
| 1 | AAV8 WT | 1 | 1 | — |
| 2 | AAV8 S279A | 3.9 | 9 | — |
| 3 | AAV2 S501 | 2 | 1 | 4.2 |
| 4 | AAV2 S671A | 9.2 | 20 | 3.4 |
| 5 | AAV2 S149A | — | — | 2 |
| 6 | AAV2 S156A | — | — | 1.8 |
| 7 | AAV2 K137R | 15 | 46 | 3.8 |
| 8 | AAV2 K143R | — | — | 1.5 |
| 9 | AAV2 K652R | — | — | 2.4 |
| 10 | AAV2 T252A | 0.1 | 0.02 | 2.7 |
| 11 | AAV2 T138A | — | — | 1 |
| 12 | AAV2 T654A | — | — | 1.8 |

DOUBLE MUTANTS

| | | | | |
|---|---|---|---|---|
| 1 | AAV8 K137R + | — | — | 2.4 |
| | S671A | | | |

### Ubiquitin Conjugation Assay and Immunoblotting:

A ubiquitination assay of viral capsids is carried out with a ubiquitin-protein conjugation kit according to the protocol of the manufacturer (Boston Biochem, Cambridge, Mass.). Briefly, 10× energy solution, conjugation fraction A, conjugation fraction B, and ubiquitin are mixed to a final reaction volume of 100 μl. The conjugation reaction is then initiated by adding 3×10⁸ heat-denatured AAV-WT, AAV S/T/K mutant vector and incubated at 37° C. for 4 hr. Equal volumes of sodium dodecyl sulfate (SDS)-denatured ubiquitinated samples are then resolved on a 4-20% gradient gel. The ubiquitination pattern for the various viral particles is detected by immunoblotting of the samples with mouse antiubiquitin monoclonal antibody (P4D1) and horseradish peroxidase (HRP)-conjugated anti-mouse IgG1 secondary (Cell Signaling Technology, Boston, Mass.). VP1, VP2, and VP3 capsid proteins are detected with AAV clone B1 antibody (Fitzgerald, North Acton, MA) and HRP-conjugated anti-mouse IgG1 secondary antibody (Cell Signaling Technology).

Result:

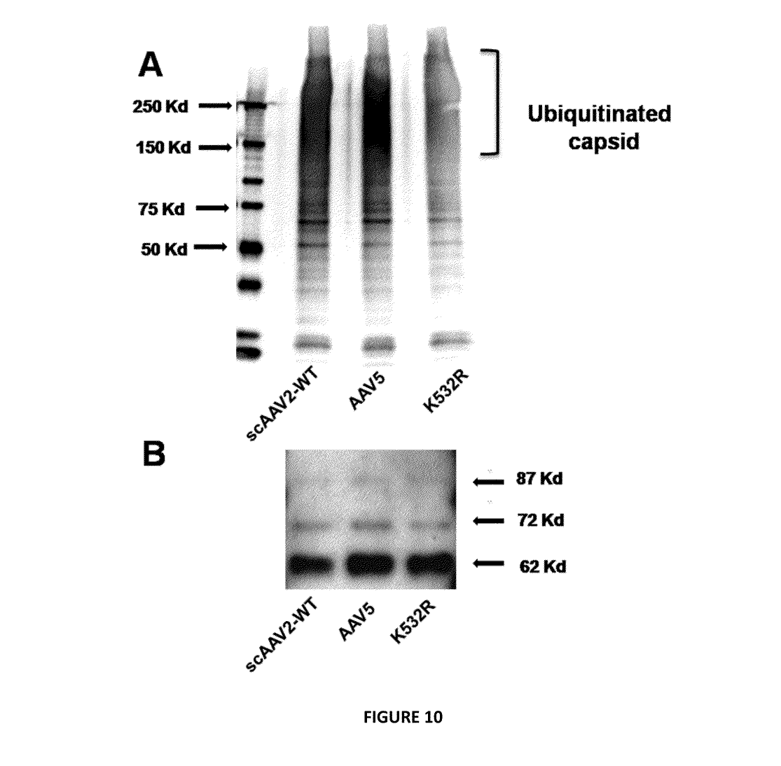

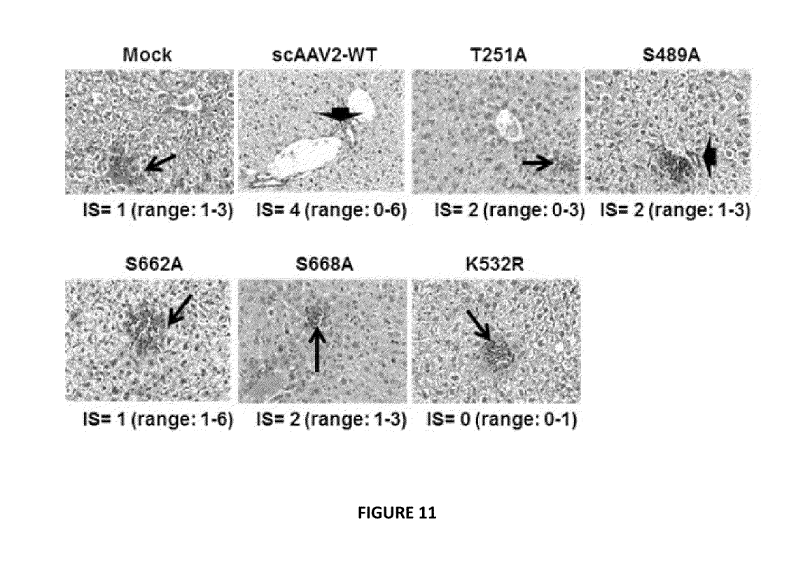

As can be seen in FIG. 10, the AAV2 K532R mutant vector demonstrated significantly reduced ubiquitination compared with either the AAV2-WT or AAV5-WT vector.

Interestingly, AAV5 capsid had higher ubiquitination than did AAV2-WT capsid, a phenomenon that has been reported previously. These data provide direct evidence that the superior transduction achieved with the AAV2 K532R mutant vector is due to reduced ubiquitination of the viral capsid, which possibly results in rapid intracellular trafficking of the virus and improved gene expression, as has been suggested previously for the AAV2 tyrosine mutant vectors.

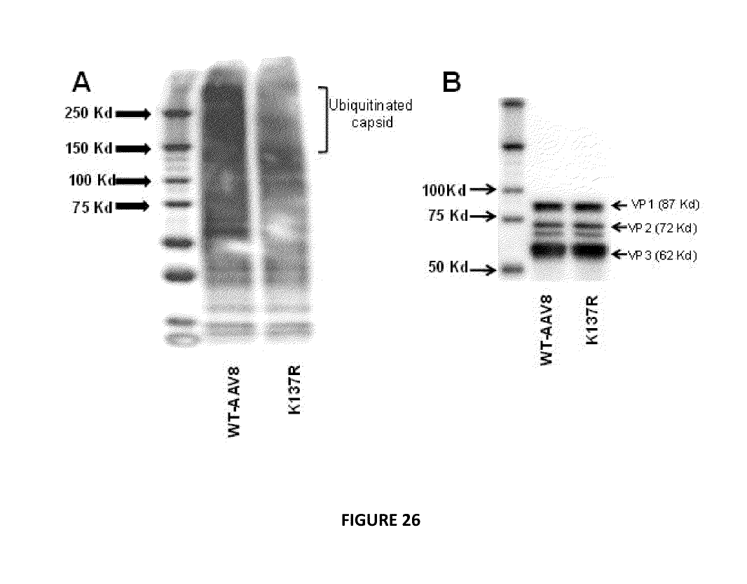

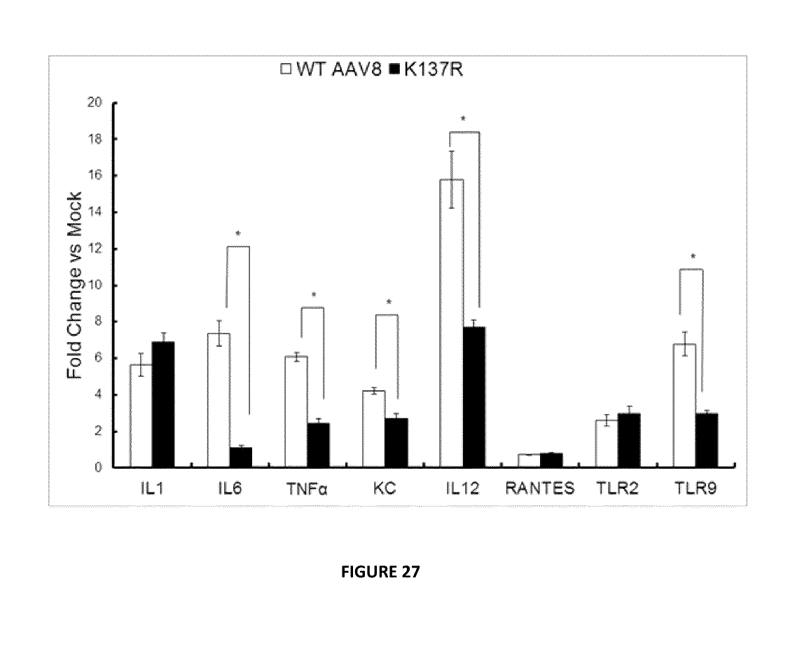

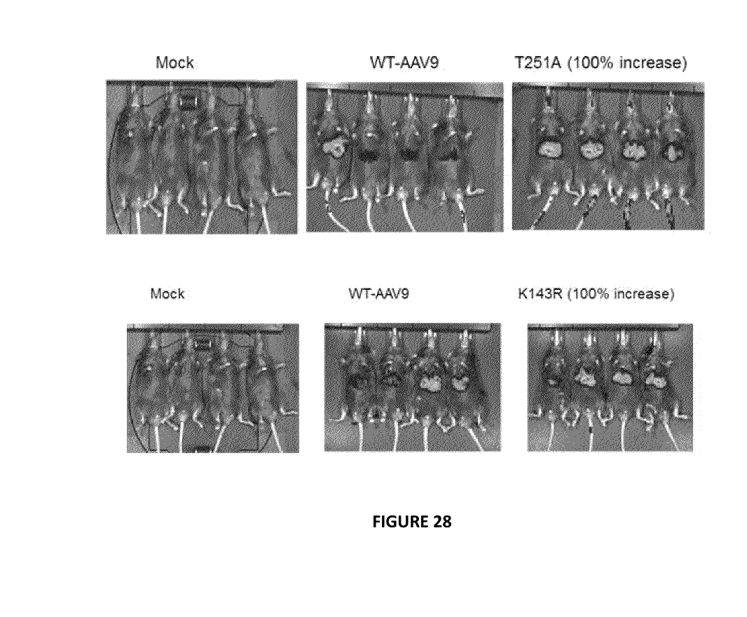

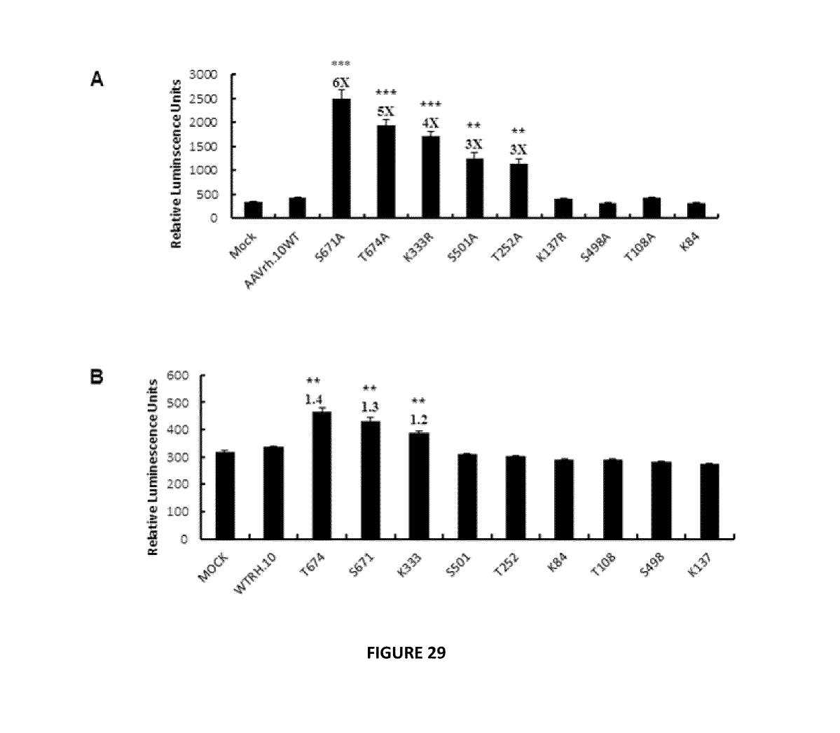

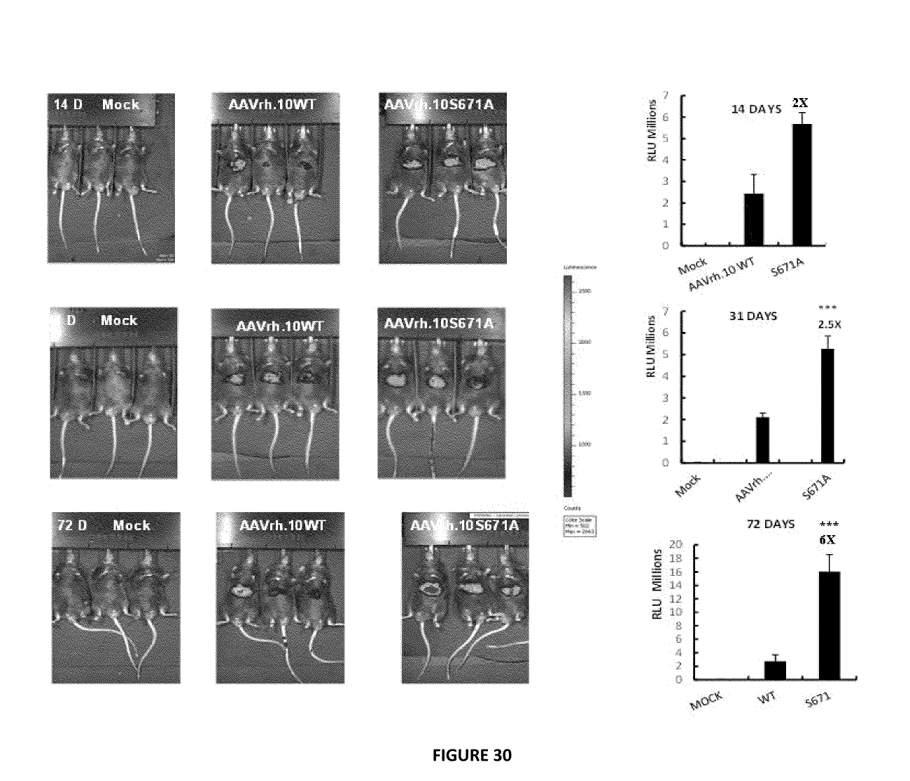

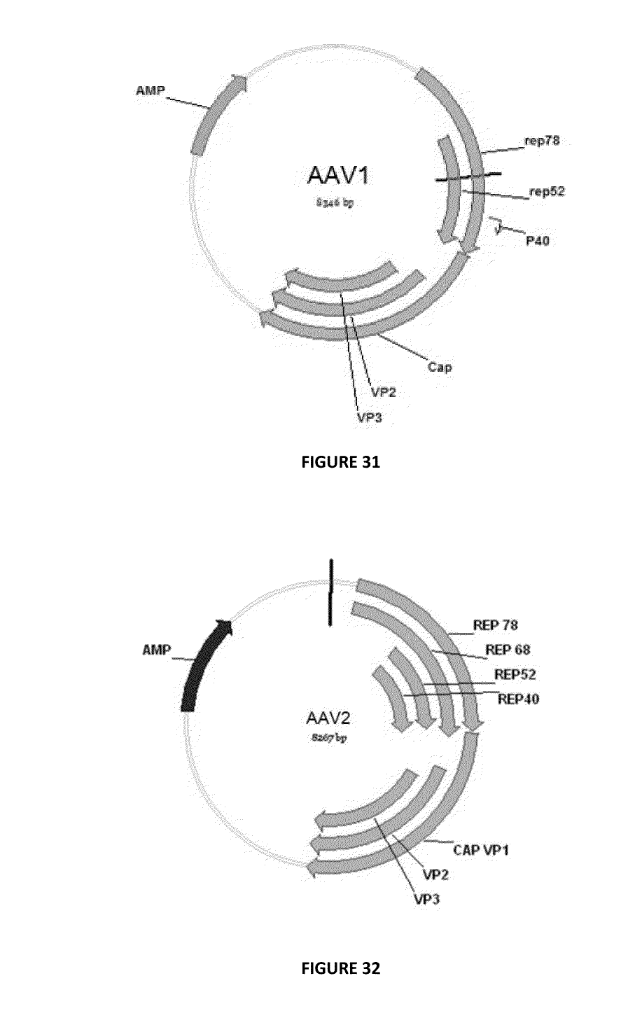

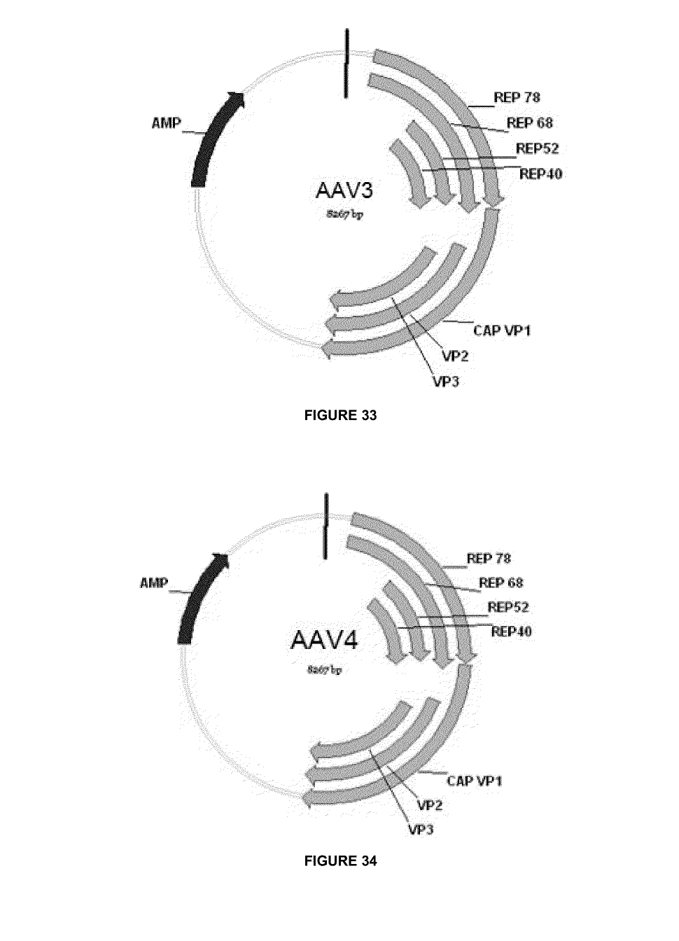

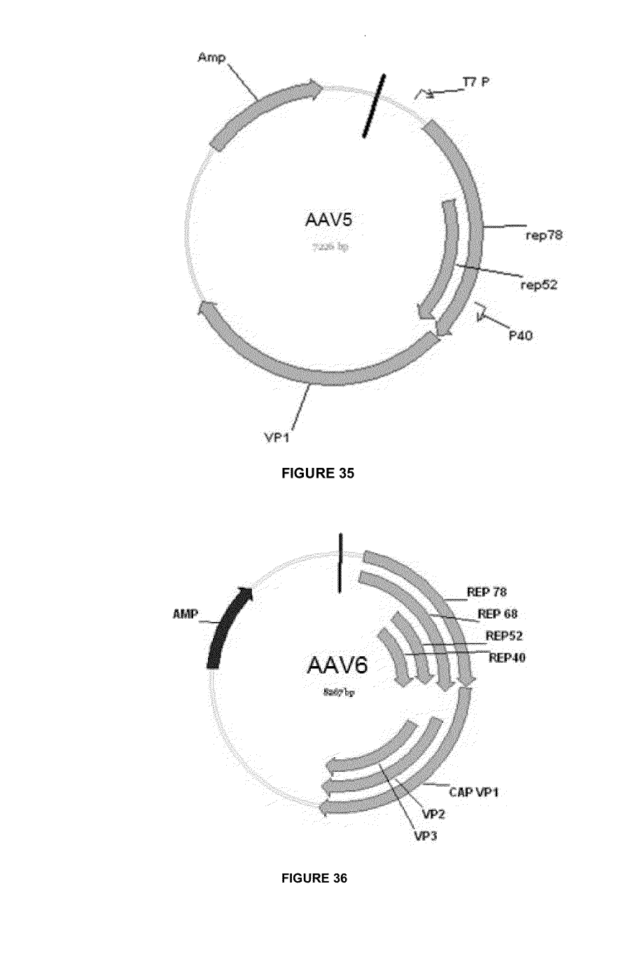

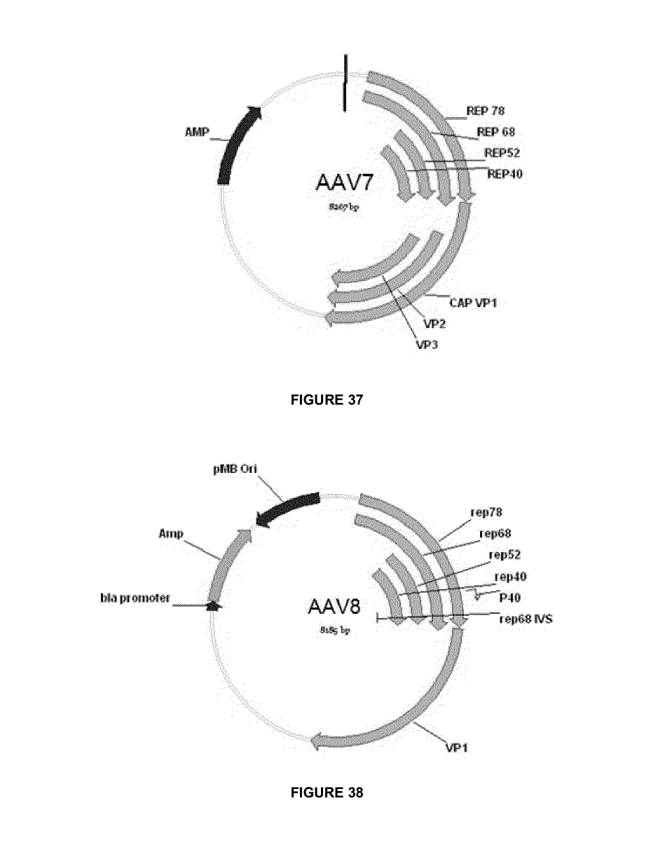

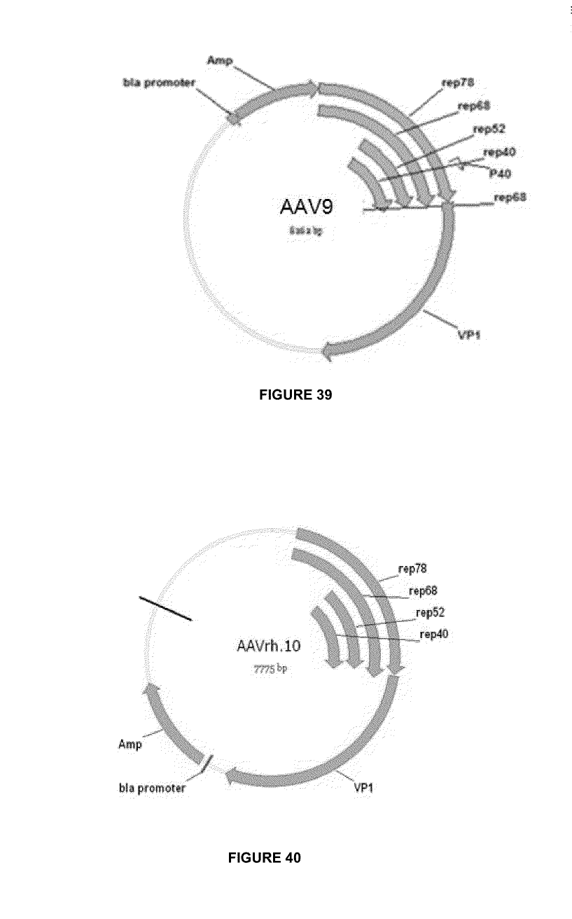

AAV8 K137R mutant vector has significantly reduced ubiquitination pattern compared to WT-AAV8 vector. AAV8 capsid proteins VP1 (87 kDa), VP2 (72 kDa), and VP3 (62 kDa) are probed as gel-loading controls, which showed similar levels of these proteins across the samples tested (FIG. 26B). These data provide direct evidence that the superior transduction achieved with the K137R mutant vector is due to the reduced ubiquitination of the viral capsid, which possibly results in rapid intracellular trafficking of the virus and improved gene expression.

We claim:

1. A nucleotide sequence selected from a group comprising SEQ ID Nos. 139 to 148, having mutation at codon selected from a group comprising TCT, TCC, TCA, TCG, AGT, AGC, ACT, ACC, ACA, ACG, AAA and AAG or any combination thereof.

2. The nucleotide sequence as claimed in claim 1, wherein the sequence selected from a group comprising SEQ ID Nos. 139 to 148, corresponds to serotypes 1 to 10 respectively, of wild type adeno-associated virus vector; and wherein the sequence after mutation is represented by SEQ ID Nos. 149 to 158, respectively with respect to the wild type SEQ ID Nos. 139 to 148.

3. The nucleotide sequence as claimed in claim 1, wherein the sequence having SEQ ID Nos. 149 to 158, corresponds to mutated serotypes 1 to 10 respectively, of adeno-associated virus vector.

4. The nucleotide sequence as claimed in claim 1, wherein the codon TCT, TCC, TCA, TCG, AGT or AGC code for amino acid serine; codon ACT, ACC, ACA or ACG code for amino acid threonine; and the codon AAA or AAG code for amino acid lysine.

5. The nucleotide sequence as claimed in claim 1, wherein the codon TCT, TCC, TCA, TCG, AGT, AGC, ACT, ACC, ACA or ACG is mutated to any codon selected from a group comprising GCT, GCC, GCA or GCG; and wherein the codon AAA or AAG is mutated to any codon selected from a group comprising CGT, CGC, CGA, CGG, AGA or AGG.

6. The nucleotide sequence as claimed in claim 5, wherein the codon GCT, GCC, GCA or GCG code for amino acid alanine; and the codon CGT, CGC, CGA, CGG, AGA or AGG code for amino acid arginine.

7. The nucleotide sequence as claimed in claim 1, wherein mutation in the codon coding for serine or threonine results in replacement of said serine or threonine amino acid with alanine amino acid; and wherein mutation in the codon coding for lysine results in replacement of said lysine amino acid with arginine amino acid.

8. The nucleotide sequence as claimed in 5, wherein the mutation in codon TCT, TCC, TCA, TCG, AGT or AGC in any of the SEQ ID Nos. 139 to 148, occurs at position of the corresponding amino acid sequence, said position selected from a group comprising 149, 156, 268, 276, 277, 278, 279, 485, 489, 490, 492, 498, 499, 501, 525, 526, 537, 547, 652, 658, 662, 663, 668, 669 and 671 or any combination thereof; and wherein the mutation in codon ACT, ACC, ACA or ACG in any of the SEQ ID Nos. 139 to 148, occurs at position of the corresponding amino acid sequence, said position selected from a group comprising 107, 108, 138, 245, 251, 252, 328, 454, 503, 654, 671, 674, 701, 713 and 716 or any combination thereof; and wherein the mutation in codon AAA or AAG in any of the SEQ ID Nos. 139 to 148, occurs at position of the corresponding amino acid sequence, said position selected from a group comprising 32, 39, 84, 90, 137, 143, 161, 333, 490, 507, 527, 532, 544 and 652 or any combination thereof.

9. A nucleotide sequence selected from a group comprising SEQ ID Nos. 70 to 138, having mutation at codon selected from a group comprising TCT, TCC, TCA, TCG, AGT, AGC, ACT, ACC, ACA, ACG, AAA and AAG or any combination thereof.

10. The nucleotide sequence as claimed in claim 9, wherein the sequence selected from a group comprising SEQ ID Nos. 70 to 138, represents wild type primer sequence capable of amplifying nucleotide sequence corresponding to serotypes 1 to 10, of wild type adeno-associated virus vector.

11. The nucleotide sequence as claimed in claim 9, wherein the codon TCT, TCC, TCA, TCG, AGT, AGC, ACT, ACC, ACA or ACG is mutated to any codon selected from a group comprising GCT, GCC, GCA or GCG; and wherein the codon AAA or AAG is mutated to any codon selected from a group comprising CGT, CGC, CGA, CGG, AGA or AGG.

12. The nucleotide sequence as claimed in claim 9, wherein the sequence having said mutation is selected from a sequence having SEQ ID Nos. 1 to 69; and wherein the mutated sequence act as primer for carrying out site directed mutagenesis for obtaining the mutated nucleotide sequence as claimed in claim 9.

13. A method of obtaining a nucleotide sequence selected from a group comprising SEQ ID Nos. 139 to 148, having mutation at codon selected from a group comprising TCT, TCC, TCA, TCG, AGT, AGC, ACT, ACC, ACA, ACG, AAA and AAG or any combination thereof, said method comprising act of introducing mutation in any of nucleotide sequence selected from a group comprising SEQ ID Nos. 139 to 148 through site directed mutagenesis by using the nucleotide sequence of claim 9.

14. The method as claimed in claim 13, wherein the sequence selected from a group comprising SEQ ID Nos. 139 to 148, corresponds to serotypes 1 to 10 respectively, of wild type adeno-associated virus vector.

15. A method of enhancing transduction efficiency, said method comprising act of expressing a target gene in presence of a nucleotide sequence selected from a group comprising SEQ ID Nos. 139 to 148, having mutation at codon selected from a group comprising TCT, TCC, TCA, TCG, AGT, AGC, ACT, ACC, ACA, ACG, AAA and AAG or any combination thereof.

16. The method as claimed in claim 15, wherein the sequence selected from a group comprising SEQ ID Nos. 139 to 148, corresponds to serotypes 1 to 10 respectively, of wild type adeno-associated virus vector.

17. The method as claimed in claim 15, wherein the transduction efficiency is enhanced with minimizing immunological response when compared with transduction carried out in presence of wild type adeno-associated virus vector having sequence selected from a group comprising SEQ ID Nos. 139 to 148.

18. A recombinant cell comprising the nucleotide sequence of claim 1.

19. A method of obtaining the recombinant cell as claimed in claim 18, said method comprising act of introducing the nucleotide sequence of claim 18 to a host cell, to obtain said recombinant cell.

20. A kit comprising a nucleotide sequence selected from a group comprising SEQ ID Nos. 70 to 138, having mutation at codon selected from a group comprising TCT, TCC, TCA, TCG, AGT, AGC, ACT, ACC, ACA, ACG, AAA and AAG or any combination thereof.
